(19) United States
(12) Patent Application Publication
(10) Pub. No.: US 2022/0130549 A1
(43) Pub. Date: Apr. 28, 2022

(54) TUMOR CLASSIFICATION BASED ON PREDICTED TUMOR MUTATIONAL BURDEN

(71) Applicant: Roche Sequencing Solutions, Inc., Pleasanton, CA (US)

(72) Inventors: Hugo Y. K. Lam, Sunnyvale, CA (US); Marghoob Mohiyuddin, Foster City, CA (US); Lijing Yao, Foster City, CA (US)

(21) Appl. No.: 17/304,547

(22) Filed: Jun. 22, 2021

Related U.S. Application Data
(63) Continuation of application No. PCT/EP2019/086781, filed on Dec. 20, 2019.
(60) Provisional application No. 62/784,486, filed on Dec. 23, 2018.
(60) Provisional application No. 62/822,690, filed on Mar. 22, 2019.

(51) Int. Cl. G16H 50/30; G16B 20/00; G16B 40/20

(52) U.S. Cl. CPC G16H 50/30; G16B 5/20; G16B 40/20; G16B 20/00

(57) ABSTRACT

The present disclosure provides systems and methods of classifying and/or identifying a cancer subtype. The present disclosure also provides methods of enhancing the prediction of a tumor mutational burden by using both synonymous and non-synonymous somatic mutations in the computation method. It is believed that by increasing the number of mutations in the computation of the tumor mutational burden, a comparatively more consistent tumor mutational burden may be derived, especially for targeted-panel sequencing. It is believed that the consistent computation of the tumor mutational burden from targeted panels allows for computationally quicker and less costly analysis of sequencing data as compared with a tumor mutational burden computed from whole exome sequencing data.

### CROSS REFERENCE TO RELATED APPLICATIONS

This application is a continuation of International Patent Application No. PCT/EP2019/086781, filed Dec. 20, 2019, which claims priority to U.S. Provisional Application No. 62/784,486, filed Dec. 23, 2018, and U.S. Provisional Application No. 62/822,690, filed Mar. 22, 2019, each of which is herein incorporated by reference in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the invention relate generally to systems and methods of tumor classification, and more specifically to systems and methods of tumor classification based on tumor mutational burden.

### BACKGROUND OF THE DISCLOSURE

Studies of human genetic variation using DNA sequencing have undergone an extraordinary development from their introduction over 40 years ago up to current technologies, which allow for a human genome to be sequenced and analyzed within a matter of days. The release of the first “next-generation sequencing” (NGS) instruments in the mid-2000s led to a revolution in disease study, offering vastly improved speed at significantly lower cost—enabling the generation of a whole human genome sequence in a matter of weeks. In addition to price and performance, the new sequencing technology also proved to compensate for some of the technical weaknesses of the older sequencing and genotyping technologies, allowing for the genome-wide detection of variants, including novel ones, at a low cost. A further breakthrough for NGS in human genomics arrived with the introduction of targeted enrichment methods, allowing for selective sequencing of regions of interest, thereby dramatically reducing the amount of sequences that needed to be generated. The approach is based on a collection of DNA or RNA probes representing the target sequences in the genome, which can bind and extract the DNA fragments originating from targeted regions.

Whole exome sequencing (WES), which enables sequencing of all protein-coding regions in the human genome (the exome) quickly became the most widely used targeted enrichment method, especially for monogenic (“Mendelian”) diseases. This approach enabled the detection of both exonic (coding) as well as splice-site variants, while requiring only approximately 2% of sequencing “load” compared to whole genome sequencing. The unbiased analysis of all genes eliminated the need for a time-consuming selection of candidate genes prior to sequencing. It has been estimated that the exome harbors about 85% of mutations with large effects on disease-related traits. In addition, exonic mutations were shown to cause the majority of monogenic diseases, with mis sense and nonsense mutations alone accounting for approximately 60% of disease mutations. (see Petersen et al., Opportunities and Challenges of Whole-Genome and -Exome Sequencing, BMC Genet. 2017; 18:14).

Recent advances in genome sequencing technologies provide unprecedented opportunities to characterize individual genomic landscapes and identify mutations relevant for diagnosis and therapy. Indeed, in recent years, NGS has also been increasingly applied for addressing pharmacogenomic research questions. It is not only possible to detect genetic causes that explain why some patients do not respond to a certain drug, but also try to predict a drug's success based on genetic information. Certain genetic variants can affect the activity of a particular protein and these can be used to estimate the probable efficacy and toxicity of a drug targeting such a protein. NGS therefore has applications far beyond finding disease-causing variants.

About 99.5% of all DNA is shared across all humans; it is the 0.5% that makes all the difference. Genetic variations, or variants, are the differences that make each person's genome unique. DNA sequencing identifies an individual's variants by comparing the DNA sequence of an individual to the DNA sequence of a reference genome maintained by the Genome Reference Consortium (GRC). It is believed that the average human's genome has millions of variants. Some variants occur in genes, but most occur in DNA sequences outside of genes. A small number of variants have been linked with diseases, but most variants have unknown effects. Some variants contribute to the differences between humans, such as different eye colors and blood types. As more DNA sequence information becomes available to the research community, the effects of some variants may be better understood.

Recent clinical trials of immunotherapy targeting immune checkpoint inhibitors have shown remarkable clinical benefits for various cancers, including Melanoma, non-small cell lung cancer (NSCLC), bladder cancer, head and neck cancer, and colorectal cancer. Blockage of programmed cell death-1 receptor (PD-1) or programmed cell death-ligand 1 (PD-L1) is one of the most studied immune checkpoint therapies. Multiple anti-PD-L1 antibodies including atezolizumab, nivolumab and pembrolizumab have been approved by FDA for melanoma and NSCLC patients. While these immune checkpoint blockade cancer therapies have dramatically improved the efficacy of immunotherapy, only a fraction of patients are responsive to the treatment. Therefore, to maximize the therapeutic benefits, it is critical to identify predictive biomarkers to distinguish the responsive and nonresponsive patients. (see Wolchok, J. D. et al. Overall Survival with Combined Nivolumab and Ipilimumab in Advanced Melanoma. N. Engl. J. Med. 377, 1345-1356 (2017); Robert, C. et al. Ipilimumab plus dacarbazine for previously untreated metastatic melanoma. N. Engl. J. Med. 364, 2517-2526 (2011); Borghaei, H. et al. Nivolumab versus Docetaxel in Advanced Nonsquamous Non-Small-Cell Lung Cancer. N. Engl. J. Med. 373, 1627-1639 (2015); Goldberg, S. B. et al. Pembrolizumab for patients with melanoma or non-small-cell lung cancer and untreated brain metastases: early analysis of a non-randomised, open-label, phase 2 trial. The Lancet Oncology 17, 976-983 (2016); Aggen, D. H. & Drake, C. G. Biomarkers for immunotherapy in bladder cancer: a moving target. 1-13 (2017). doi:10.1186/s40425-017-0299-1; Saleh, K., Eid, R., Haddad, F. G., Khalife-Saleh, N. & Kourie, H. R. New developments in the management of head and neck cancer—impact of pembrolizumab. TCRM Volume 14, 295-303 (2018); FDA fast tracks nivolumab for advanced non-squamous non-small cell lung cancer. The Pharmaceutical Journal (2015). doi:10.1211/pj.2015.20069525; Jean, F., Tomasini, P. & Barlesi, F. Atezolizumab: feasible second-line therapy for patients with non-small cell lung cancer? A review of efficacy, safety and place in therapy. Ther Adv Med Oncol 9, 769-779(2017).

Multiple studies have shown that the PD-L1 expression level, microsatellite instability-high (MSI-H) and mismatch repair deficient (dMMR) could be predictive biomarkers for the clinical outcome of anti-PD-L1 therapy. Currently, PD-L1 immunohistochemistry (IHC) has been developed as companion or complementary diagnostic assay for anti-PD-L1 therapy. MSI-H and dMMR are also FDA approved biomarkers for the use of anti-PD1 cancer treatment. Tumor mutational burden-high (TMB-H) has been shown to be another emerging biomarker for anti-PD-L1 treatment. The underlying hypothesis is that more neoantigens from hypermutated tumor lead to stronger adaptive immune response. (see Reck, M. et al. Pembrolizumab versus Chemotherapy for PD-L1-Positive Non-Small-Cell Lung Cancer. N. Engl. J. Med. 375, 1823-1833 (2016); Le, D. T. et al. PD-1 Blockade in Tumors with Mismatch-Repair Deficiency. N. Engl. J. Med. 372, 2509-2520 (2015); Chalmers, Z. R. et al. Analysis of 100,000 human cancer genomes reveals the landscape of tumor mutational burden. 1-14 (2017)).

Tumor mutational burden (TMB) is a measure of the number of mutations carried by tumor cells and an emerging area of focus in biomarker research. By comparing DNA sequences from a patient's healthy tissues and tumor cells, and using a number of complex algorithms, the number of acquired somatic mutations present in tumors, but not in normal tissues, may be determined. Unlike most cancer biomarkers for immunotherapies, which are specific to certain immune proteins expressed by the tumor, TMB is derived solely from mutations. It is believed that some tumors with a higher number of mutations may be more susceptible to an immune response. (see Chalmers, Z. R. et al. Analysis of 100,000 human cancer genomes reveals the landscape of tumor mutational burden. 1-14 (2017). doi:10.1186/s13073-017-0424-2; Friends of Cancer Research: https://www.focr.org/tmb; Matthew D. Hellmann et al. Nivolumab (nivo)+ipilimumab (ipi) vs platinum-doublet chemotherapy (PT-DC) as first-line (1L) treatment (tx) for advanced non-small cell lung cancer (NSCLC): initial results from CheckMate 227, AACR 2018).

### BRIEF SUMMARY OF THE DISCLOSURE

The level of programmed death-ligand 1 expression detected by immunohistochemistry, on the surface of tumor cells, is so far, the unique validated biomarker for checkpoint inhibitors therapies anti programmed cell death-1 or PD-L1 in cancer, such as lung cancer. PD-L1 expression alone, however, is often insufficient for patient selection in several tumor types. Recently, new insights have focused the important role of the tumor mutational burden in this setting. The tumor genome is believed to be a driver of anti-cancer immunity and, depending on the tumor mutational burden, the response to immunotherapy varies, suggesting that neoantigens generated by these mutations are crucial targets for T cells in cancer immunity. Tumor mutational burden is thus a highly relevant tool which may be used to evaluate a patient's sensitivity to immunotherapy.

Tumor mutational burden is a measure of the quantity of somatic mutations in a tumor and the well-adopted calculation standard is the determination of the number of non-synonymous somatic mutations per megabase by whole exome sequencing. Several problems, however, currently make it difficult to use TMB as a clinical-decision-making biomarker. It is believed that one shortcoming is the inconsistency of TMB calculated using whole exome sequencing and various next-generation sequencing targeted panels (the need for targeted panels arises due to the relatively high cost of whole exome sequencing). One possible source for the variability is the design of the targeted panels for cancers which are believed to be enriched with cancer driver mutations and mutation hot spots. This, is believed, may cause an over-estimation of the mutation rate. While various filtering strategies may be applied to remove such driver mutations (e.g. COSMIC may be used to reduce driver mutations), it is believed, however, that the use of these additional filters may further contribute to inconsistencies in the calculation.

It is believed that another shortcoming is that there is no statistical cutoff to define TMB-high patients, to differentiate them from TMB-low patients. Multiple arbitrary thresholds such as 10 or 20/Mb have been used in various research articles and clinical trials, but these arbitrary thresholds may not be coincident for all tumor types; and clinical cut-offs should be accurately established for each cancer type in order to translate the use of TMB biomarker into clinical practice. This is a technical problem and the presently disclosed systems and methods overcome this inherently technological problem, such as by developing a computer system (including a sequencing system) and/or method which enables the estimation of a tumor mutational burden without using arbitrary cutoffs while, at the same time, incorporating additional sequencing data (e.g. additional mutation data) into the solution. Applicant has been able to do so without increasing the computational burden, i.e. there is no increased computation burden using the processes described herein despite using an increased amount of sequencing data into the TMB computation. Applicant also submits that the solution proposed herein enables TMB estimations for panels which are superior to counting methods (described herein), since the presently disclosed methods are comparatively more consistent than TMB estimation by the counting method, while not being computationally burdensome. It is also believed that driver mutation effects may be systematically removed by using both synonymous and non-synonymous somatic mutations in the tumor mutational burden computation method.

In view of the foregoing, and in one aspect of the present disclosure, Applicant has developed a method of identifying clear cutoffs in tumor mutational burden data. In some embodiments, is a method of identifying at least two cancer subtypes comprising (i) performing a data transformation on an estimated tumor mutational burden, and (ii) modeling the transformed estimated tumor mutational burden using a Gaussian mixture model, where each K^{th} component of the Gaussian mixture model represents one cancer subtype. In some embodiments, the data transformation is a log-transformation. In some embodiments, the transformed tumor mutational burden identifies at least three different cancer subtypes, each having distinguishable mutation profiles. In some embodiments, the three cancer subtypes are identified for each of colorectal cancer, stomach cancer, and endometrial cancer. In some embodiments, the tumor mutational burden is estimated using identified non-synonymous mutations and identified synonymous mutations. In some embodiments, the tumor mutational burden is estimated by performing a maximum likelihood estimation using identified non-synonymous and synonymous mutations and a plurality of pre-determined mutation rate parameters.

In another aspect of the present disclosure is a method of estimating tumor mutational burden comprising: (a) identifying genetic alterations in sequencing data; and (b) performing a maximum likelihood estimation using the identified genetic alterations and a plurality of pre-determined mutation rate parameters, such as parameters derived from a training cohort. In some embodiments, the genetic alterations comprises non-synonymous and synonymous mutations. It is believed that the combined use of synonymous and non-synonymous mutations increases the number of mutations per tumor mutational burden calculation and helps to remove driver gene effects (see also PCT Publication No. WO2017/181134, the disclosure of which is hereby incorporated by reference herein in its entirety). In some embodiments, the method further comprises computing a data transformation of the estimated tumor mutational burden. In some embodiments, the data transformation comprises conforming data to normality, e.g. conforming positively skewed data to normality. In some embodiments, the data transformation comprises a method which reduces variability. In some embodiments, the data transformation comprises calculating a log transform of the estimated tumor mutational burden. In some embodiments, the method further comprises classifying a cancer subtype based on a modeling of the log-transformed estimated tumor mutational burden.

In some embodiments, the sequencing data is training data, and the estimated tumor mutational burden is used to identify cancer subtypes (such as new cancer subtypes) within the training data, e.g. training data for a specific type of cancer. For example, the training data may be used to identify three different cancer subtypes within training data (e.g., whole exome sequencing data that is publicly available). In some embodiments, the identified three different cancer subtypes include “low TMB,” “high TMB,” and “extreme TMB.”

In some embodiments, the sequencing data is test data, i.e., sequencing data derived from a biological sample derived from a patient, and the estimated tumor mutational burden is utilized to classify the biological sample as having one of a plurality of different pre-determined cancer subtypes, e.g. “low TMB,” “high TMB,” and “extreme TMB.” In some embodiments, the method further comprises administering an immunotherapy to the patient if the biological sample is classified as either “high TMB” or “extreme TMB.” In some embodiments, the immunotherapy is a checkpoint inhibitor. In some embodiments, the immunotherapy is an anti-PD-1 antibody. In some embodiments, the anti-PD-1 antibody is selected from nivolumab (also known as OPDIVO®) or pembrolizumab (Merck; also known as KEYTRUDA®, lambrolizumab, see WO2008/156712). Other suitable anti-PD-1 antibodies are disclosed in PCT Publication Nos. WO 2015/112900, WO 2012/145493, WO 2015/112800, WO2014/179664, WO 2015/085847, WO 2017/040790, WO 2017/024465, WO 2017/025016, WO 2017/132825, and WO 2017/133540, the disclosures of which are hereby incorporated by reference herein in their entireties.

In another aspect of the present disclosure is a system for classifying a tumor sample derived from a patient, the system comprising: (i) one or more processors, and (ii) one or more memories coupled to the one or more processors, the one or more memories to store computer-executable instructions that, when executed by the one or more processors, cause the system to perform operations comprising: receiving an identification of somatic mutations within obtained sequencing data, the sequencing data derived from the tumor sample; estimating a tumor mutational burden based on the received identified somatic mutations; and assigning a cancer subtype to the tumor sample based on a log-transform of the estimated tumor mutational burden. In some embodiments, the log-transform of the estimated tumor mutational burden is derived by computing a log of the estimated tumor mutational burden (e.g. computing a natural log, a log(1), a log(2), etc.). It is believed that this is a technological solution to an inherently technological problem and the system described herein provides a solution to improving the classification of a tumor sample derived from sequencing data and/or reducing the computational burden associated with classifying a tumor sample using sequencing data derived from WES.

In another aspect of the present disclosure is a method of classifying a tumor sample derived from a patient comprising: acquiring sequencing data derived from nucleic acids in the tumor sample; identifying somatic mutations within the acquired sequencing data the sample; estimating a tumor mutational burden based on the identified somatic mutations; computing a log-transform of the estimated tumor mutational burden to provide a log-transformed estimated tumor mutational burden; and assigning a cancer subtype to the tumor sample based on the log-transformed estimated tumor mutational burden. In some embodiments, the assignment of the cancer subtype comprises (i) modeling the log-transformed estimated tumor mutational burden as a Gaussian mixture model, where each K^{th} component of the Gaussian mixture model represents one cancer subtype; (ii) computing an assignment score for each K^{th} component of the Gaussian mixture model; (iii) identifying a K^{th} component having a highest assignment score; and (iv) assigning the cancer subtype associated with the identified K^{th} component having the highest assignment score as the cancer subtype of the tumor sample. In some embodiments, parameters for each K^{th} component are estimated using an expectation-maximization algorithm based on training data, e.g. publicly available training data representing a population of patients having a specific type of cancer.

In some embodiments, the tumor mutational burden is estimated using identified non-synonymous mutations. In some embodiments, the tumor mutational burden is estimated by dividing a total number of identified non-synonymous mutations by a pre-determined genome size.

In some embodiments, the tumor mutational burden is estimated using identified non-synonymous mutations and identified synonymous mutations. In some embodiments, the tumor mutational burden is estimated by performing a maximum likelihood estimation using the identified non-synonymous and synonymous mutations and a plurality of pre-determined mutation rate parameters. In some embodiments, the plurality of pre-determined mutation rate parameters comprise (i) gene-specific mutation rate factors, and (ii) context-specific mutation rates. In some embodiments, the context-specific mutation rates are selected from the group consisting of (i) tri-nucleotide context specific mutation rates; (ii) di-nucleotide context specific mutation rates, and; (iii) mutation signatures. In some embodiments, the plurality of pre-determined mutation rate parameters are derived by modeling an observed number of mutations for each gene in a training sample derived from whole-exome sequencing. In some embodiments, the modeling is performed using a regression model and a maximum likelihood algorithm within a Bayesian framework.

In some embodiments, the pre-determined mutation rate parameters are derived by: (i) estimating a background mutation rate using one of a negative binomial regression, a poisson regression, a zero-inflated poisson regression, or a zero-inflated negative binomial regression with consideration of only known influencing factors; (ii) estimating a background mutation rate using single gene analysis with consideration of unknown influencing factors; and (iii) combining the estimates of (i) and (ii) within a Bayesian framework. In some embodiments, the zero-inflated poisson regression is used for estimation of the background mutation rate with consideration of only known influencing factors.

In some embodiments, the method further comprises computing an overall survival based on the cancer subtype assigned to the tumor sample. In some embodiments, the method further comprises computing a progression free survival based on the cancer subtype assigned to the tumor sample. In some embodiments, the method further comprises administering a therapeutic based on the cancer subtype assigned to the tumor sample. In some embodiments, the therapeutic is an immunotherapy (e.g. an anti-PD1 antibody). In some embodiments, the immunotherapy is a checkpoint inhibitor.

In some embodiments, the sequencing data for the tumor sample is derived from whole exome sequencing or targeted panel sequencing of nucleic acids derived from the tumor sample. In some embodiments, the cancer subtypes are low TMB, high TMB, and extreme TMB. In some embodiments, the extreme TMB cancer subtype comprises (i) a high single nucleotide variant mutation rate; (ii) a low INDEL mutation rate; and (iii) high non-synonymous mutations in a POLE gene. In some embodiments, the high TMB cancer subtype comprises (i) a high MSI-H rate; and (ii) a high INDEL mutation rate.

In another aspect of the present disclosure is a method of classifying a tumor sample derived from a patient comprising: performing whole exome sequencing or targeted panel sequencing on the tumor sample to derive sequencing data; identifying somatic mutations within the derived sequencing data in the sample; estimating a tumor mutational burden based on the identified somatic mutations; computing a log-transform of the estimated tumor mutational burden to provide a log-transformed estimated tumor mutational burden; and assigning a cancer subtype to the tumor sample based on the log-transformed estimated tumor mutational burden. In some embodiments, the cancer subtype is assigned by modeling the log-transformed estimated tumor mutational burden as a Gaussian mixture model. In some embodiments, each K^{th} component of the Gaussian mixture model represents one cancer subtype. In some embodiments, the tumor mutational burden is estimated using identified non-synonymous mutations and identified synonymous mutations. In some embodiments, the tumor mutational burden is estimated by performing a maximum likelihood estimation using the identified non-synonymous and synonymous mutations and a plurality of pre-determined mutation rate parameters. In some embodiments, the plurality of pre-determined mutation rate parameters comprise (i) gene-specific mutation rate factors, and (ii) context-specific mutation rates. In some embodiments, the pre-determined mutation rate parameters are derived by: (i) estimating a background mutation rate using one of a negative binomial regression, a poisson regression, a zero-inflated poisson regression, or a zero-inflated negative binomial regression with consideration of only known influencing factors; (ii) estimating a background mutation rate using single gene analysis with consideration of unknown influencing factors; and (iii) combining the estimates of (i) and (ii) within a Bayesian framework.

In another aspect of the present disclosure is a method of treating a subject afflicted with a tumor comprising: (i) identifying a cancer subtype based on tumor mutational burden; and (ii) administering to the subject a therapeutically effective amount of an antibody or an antigen-binding portion thereof that binds specifically to a PD-1 receptor and inhibits PD-1 activity; wherein the cancer subtype is identifying by acquiring sequencing data for the tumor sample; identifying somatic mutations within the acquired sequencing data in the sample; estimating a tumor mutational burden based on the identified somatic mutations; computing a log-transform of the estimated tumor mutational burden to provide a log-transformed estimated tumor mutational burden; and assigning a cancer subtype to the tumor based on the log-transformed estimated tumor mutational burden; wherein the therapeutically effective amount of the antibody or the antigen-binding portion thereof that binds specifically to a PD-1 receptor and inhibits PD-1 activity is administered if the cancer subtype assigned to the tumor is “high TMB” or “extreme TMB.” In some embodiments, the “extreme TMB” cancer subtype comprises (i) a high single nucleotide variant mutation rate; (ii) a low INDEL mutation rate; and (iii) high non-synonymous mutations in a POLE gene. In some embodiments, the cancer subtype is classified by modeling the log-transformed estimated tumor mutational burden as a Gaussian mixture model. In some embodiments, the somatic mutations comprises non-synonymous and synonymous mutations.

In another aspect of the present disclosure is a method of classifying a tumor sample derived from a patient comprising: obtaining sequencing data for the tumor sample; identifying somatic mutations within the obtained sequencing data; estimating a tumor mutational burden based on the identified somatic mutations; computing a transformation of the estimated tumor mutational burden to provide a transformed estimated tumor mutational burden; and assigning a cancer subtype to the tumor sample based on the transformed estimated tumor mutational burden. In some embodiments, the computing of the transformation of the estimated tumor mutational burden comprises calculating a log transform of the estimated tumor mutational burden. In some embodiments the log transform is selected from a natural log, log(10), or log(2).

In another aspect of the present disclosure is a system for classifying a tumor sample derived from a patient, the system comprising: (i) one or more processors, and (ii) one or more memories coupled to the one or more processors, the one or more memories to store computer-executable instructions that, when executed by the one or more processors, cause the system to perform operations comprising: receiving an identification of somatic mutations within acquired sequencing data within the tumor sample; estimating a tumor mutational burden based on the received identified somatic mutations; computing a log-transform of the estimated tumor mutational burden to provide a log-transformed estimated tumor mutational burden; and assigning a cancer subtype to the tumor sample based on the log-transformed estimated tumor mutational burden.

In some embodiments, the assignment of the cancer subtype comprises (i) modeling the log-transformed estimated tumor mutational burden as a Gaussian mixture model, where each K^{th} component of the Gaussian mixture model represents one cancer subtype; (ii) computing an assignment score for each K^{th} component of the Gaussian mixture model; (iii) identifying a K^{th} component having a highest assignment score; and (iv) assigning the cancer subtype associated with the identified K^{th} component having the highest assignment score as the cancer subtype of the tumor sample. In some embodiments, the parameters for each K^{th} component are estimated using an expectation-maximization algorithm based on training data.

In some embodiments, the tumor mutational burden is estimated using identified non-synonymous mutations. In some embodiments, the tumor mutational burden is estimated by dividing a total number of identified non-synonymous mutations by a pre-determined genome size.

In some embodiments, the tumor mutational burden is estimated using identified non-synonymous mutations and identified synonymous mutations. In some embodiments, the tumor mutational burden is estimated by performing a maximum likelihood estimation using the identified non-synonymous and synonymous mutations and a plurality of pre-determined mutation rate parameters. In some embodiments, the plurality of pre-determined mutation rate parameters comprise (i) gene-specific mutation rate factors, and (ii) context-specific mutation rates. In some embodiments, the context-specific mutation rates are selected form the group consisting of (i) tri-nucleotide context specific mutation rates; (ii) di-nucleotide context specific mutation rates, and; (iii) mutation signatures.

In some embodiments, the plurality of pre-determined mutation rate parameters are derived by modeling an observed number of mutations for each gene in a training sample derived from whole-exome sequencing. In some embodiments, the pre-determined mutation rate parameters are derived by: (i) estimating a background mutation rate using one of a negative binomial regression, a poisson regression, a zero-inflated poisson regression, or a zero-inflated negative binomial regression with consideration of only known influencing factors; (ii) estimating a background mutation rate using single gene analysis with consideration of unknown influencing factors; and (iii) combining the estimates of (i) and (ii) within a Bayesian framework. In some embodiments, the zero-inflated poisson regression is used for estimating the background mutation rate with consideration of only known influencing factors. In some embodiments, the zero-inflated negative binomial regression is used for estimating of the background mutation rate with consideration of only known influencing factors.

In some embodiments, the system further comprises instructions for computing an overall survival based on the cancer subtype assigned to the tumor sample. In some embodiments, the system further comprises instructions for computing a progression free survival based on the cancer subtype assigned to the tumor sample. In some embodiments, the received identified somatic mutations are derived from targeted panel sequencing of nucleic acids derived from the tumor sample.

In another aspect of the present disclosure is a system for identifying cancer subtypes within whole exome sequencing data for a type of cancer, the system comprising: (i) one or more processors, and (ii) one or more memories coupled to the one or more processors, the one or more memories to store computer-executable instructions that, when executed by the one or more processors, cause the system to perform operations comprising: receiving an identification of somatic mutations within acquired whole exome sequencing data; estimating a tumor mutational burden based on the received identified somatic mutations; computing a log-transform of the estimated tumor mutational burden to provide a log-transformed estimated tumor mutational burden; and identifying the cancer subtypes by modeling the log-transformed estimated tumor mutational burden as a Gaussian mixture model. In some embodiments, the tumor mutational burden is estimated using identified non-synonymous mutations and identified synonymous mutations. In some embodiments, the tumor mutational burden is estimated by performing a maximum likelihood estimation using the identified non-synonymous and synonymous mutations and a plurality of pre-determined mutation rate parameters. In some embodiments, three cancer subtypes are identified within whole exome sequencing data derived from a population of patients (e.g. patients having the same type of cancer, such as colorectal cancer, endometrial cancer, or stomach cancer), and wherein one of the three cancer subtypes comprises patients whose sequencing data has at least (i) high SNV mutation rates, and (ii) low INDEL mutation rates.

In another aspect of the present disclosure is a non-transitory computer-readable medium storing instructions for estimating a tumor mutational burden comprising: identifying non-synonymous and synonymous mutations in sequencing data; and performing a maximum likelihood estimation using the identified non-synonymous and synonymous mutations and a plurality of pre-determined mutation rate parameters. In some embodiments, the non-transitory computer-readable medium further comprises instructions for deriving the plurality of pre-determined mutation rate parameters, such as derived from training data. In some embodiments, the plurality of pre-determined mutation rate parameters are derived by modeling an observed number of mutations for each gene in a training sample derived from whole-exome sequencing. In some embodiments, the non-transitory computer-readable medium further comprises instructions for computing the log-transform of the estimated tumor mutational burden. In some embodiments, the non-transitory computer-readable medium further comprises instructions for classifying a cancer subtype based on the log-transformed estimated tumor mutational burden. In some embodiments, the classifying of the cancer subtype comprises modeling the log-transformed estimated tumor mutational burden as a Gaussian mixture model, where each K^{th} component of the Gaussian mixture model represents one cancer subtype.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a general understanding of the features of the disclosure, reference is made to the drawings. In the drawings, like reference numerals have been used throughout to identify identical elements.

FIG. 1 illustrates a system including a sequencing device networked to a computer system in accordance with some embodiments.

FIG. 2 illustrates a system having a training module and a testing module communicatively coupled to a sequencing module and/or storage system in accordance with some embodiments.

FIG. 3A sets forth a flow chart illustrating a method of predicting a cancer subtype of a new sample in accordance with some embodiments.

FIG. 3B sets forth a flow chart illustrating a method of predicting a cancer subtype of a new sample, and further illustrates the derivation of parameters for use in estimating a tumor mutational burden in accordance with some embodiments.

FIG. 4 illustrates a method of modeling a log-transformed estimated tumor mutational burden in accordance with some embodiments.

FIG. 5A provides a flowchart which illustrates a method of estimating different types of background mutation rates in accordance with some embodiments.

FIG. 5B provides a flowchart which illustrates a method of estimating different types of background mutation rates in accordance with some embodiments.

FIG. 5C provides a chart illustrating the method of subtype classification based on log-transformed TMB using GMM.

FIG. 6A provides (panel A1) distribution plot of log-transformed TMB for colorectal cancer. Three subtypes were determined by Gaussian Mixture Model classification and labeled with black (TMB-Low), orange (TMB-High) and blue (TMB-Extreme) in allClass bar. MSI status for each subject was shown with green (MSS) and red (MSI-H) in msi bar. Non-synonymous mutation existence (occurrence >1) in POLE or dMMR pathway genes, including MLH1, MLH3, MSH2, MSH3, MSH6, PMS1, PMS2 were shown in blue and wild type were shown in yellow. (panel B1) INDEL mutation rate and percentage were shown in boxplots for three subtypes. (panel C1) Non-synonymous mutation in dMMR/POLE genes and MSI status were summarized. Fisher exact tests were conducted to generate the p-value for each mutation profile among the subtypes.

FIG. 6B provides (panel A1) distribution plot of log-transformed TMB for endometrial cancer. Three subtypes were determined by Gaussian Mixture Model classification and labeled with black (TMB-Low), orange (TMB-High) and blue (TMB-Extreme) in allClass bar. MSI status for each subject was shown with green (MSS) and red (MSI-H) in msi bar. Non-synonymous mutation existence (occurrence >1) in POLE or dMMR pathway genes, including MLH1, MLH3, MSH2, MSH3, MSH6, PMS1, PMS2 were shown in blue and wild type were shown in yellow. (panel B1) INDEL mutation rate and percentage were shown in boxplots for three subtypes. (panel C1) Non-synonymous mutation in dMMR/POLE genes and MSI status were summarized. Fisher exact tests were conducted to generate the p-value for each mutation profile among the subtypes.

FIG. 6C provides (panel A1) distribution plot of log-transformed TMB for stomach cancer. Three subtypes were determined by Gaussian Mixture Model classification and labeled with black (TMB-Low), orange (TMB-High) and blue (TMB-Extreme) in allClass bar. MSI status for each subject was shown with green (MSS) and red (MSI-H) in msi bar. Non-synonymous mutation existence (occurrence >1) in POLE or dMMR pathway genes, including MLH1, MLH3, MSH2, MSH3, MSH6, PMS1, PMS2 were shown in blue and wild type were shown in yellow. (panel B1) INDEL mutation rate and percentage were shown in boxplots for three subtypes. (panel C1) Non-synonymous mutation in dMMR/POLE genes and MSI status were summarized. Fisher exact tests were conducted to generate the p-value for each mutation profile among the subtypes.

FIG. 7A illustrates the survival outcome association with three cancer subtypes. The survival curves by Kaplan-Meier analysis using aggregated colorectal, endometrial and stomach patients are shown.

FIG. 7B illustrates the survival outcome association with three cancer subtypes. The proportional hazard ratio analysis by cox proportional-hazards model is illustrated.

FIG. 8 illustrates the abundance of immune infiltrates among three subtypes.

FIGS. 9A and 9B set forth a comparison of TMB calculated by counting (in blue) or using the method proposed herein (in red) against TMB determined by the “gold standard method” in x axis. Two panels, including FMI panel (A) and AVENIO panel (B) are shown. “Gold standard” refers to the well-adopted calculation standards, which is determined by dividing the number of non-synonymous mutations (the count of the mutations) by a predefined genomic size using WES. The well-adopted calculation standards were shown in x-axis. The approach that requires the counting of the total number of mutations from pre-defined genome regions will be referred as the “counting method.” When the counting method is applied to non-synonymous mutation detected from WES, it is the current standard TMB measurement. It is believed that there exists an inconsistency between WES-based TMB and panel-based TMB when using the counting method. (WES-based TMB refers to the TMB predicted by WES data; Panel-based TMB refers to the TMB predicted by targeted panel sequencing.) FMI panel refers to targeted sequencing panel for FoundationOne CDx™ (https://www.foundationmedicine.com/genomic-testing/foundation-one-cdx). The panel contains regions from 324 genes. AVENIO P3 panel refers to targeted sequencing panel for AVENIO ctDNA Surveillance Kit (https://sequencing.roche.com/en/products-solutions/by-category/assays/ctdna-surveillance-kits.html). The panel contains regions from 197 genes.

FIG. 10A provides a landscape of driver mutations in POLE detected in the TMB-extreme group (top) compared with aggregated TMB-high and TMB-low group (bottom). An enrichment p-value using a binomial test is shown in parentheses.

FIGS. 10B and 10C provide a landscape of driver mutations in MLH3 and MSH3 detected in TMB-high group (top) compared with aggregated TMB-extreme and TMB-low group (bottom). An enrichment p-value using a binomial test is shown in parentheses.

FIG. 11 provides a series of plots showing the comparison of overall accuracy (red), overall kappa score (orange) and F1 score for each identified cancer subtype (TMB-low in cyan, TMB-high in green and TMB-extreme in blue) for TMB subtype classification using TMB predicted by Estimation and Classification of TMB) (“ecTMB”) or the counting method. F1 score is a way to measure accuracy of a test, which consider both precision and recall. The formula is F1=2*(precision*recall)/(precision+recall)

FIGS. 12A and 12B provide plots which show the comparisons of model accuracy between the GLM model and a final (3-steps) approach in training sets (FIG. 12A) and in testing sets (FIG. 12B). RMSE, MAE and R-squared were calculated between predicted number of synonymous mutations and observed value for each gene in each sample (top) and each gene in aggregated samples (bottom).

FIGS. 12C, 12D, and 12E illustrate the predicted number of background synonymous (top)/non-synonymous (bottom) mutations of each gene plotted against observed mutations in colorectal (FIG. 12C), stomach (FIG. 12D) and endometrial (FIG. 12E) cancers. The prediction made by the GLM model was labeled in cyan and final (3-steps) approach in yellow. Several well-known driver genes were circled and labeled in FIGS. 12C, 12D, and 12E.

FIG. 13A provides a plot which shows the comparisons of prediction accuracy when different proportions of non-synonymous mutations were used. RMSE, MAE and correlation coefficients were calculated between predicted TMB and standard WES-based TMB before log-transformation (top) and after log-transformation (bottom).

FIG. 13B illustrates biases, upper limits, and lower limits when various proportions of non-synonymous mutation were used for TMB estimation. The results using the non-log-transformation value (top) and log-transformation (bottom) are both shown. The middle circle indicates the bias (mean difference) and the two solid lines around it are the 95% confidence intervals for the bias. The two dotted lines on the top are 95% confidence intervals for the upper limit of 95% agreement; the dotted lines on the bottom are 95% confidence intervals for the lower limit of 95% agreement. Biases, upper limits and low limits were determined by Bland-Altman analysis.

FIG. 13C illustrates the predicted TMB as plotted against a standard WES-based TMB calculation before log-transformation (top) and after log-transformation (bottom). The linear regression lines were added. Standard WES-based TMB was calculated by counting the number of non-synonymous mutations and then dividing by size of the exome.

FIG. 14A provides plots which show comparisons of prediction accuracy when different proportions of non-synonymous mutation were used for each cancer and each panel. RMSE, MAE, and correlation coefficients were calculated between the predicted panel-based TMB and standard WES-based TMB before log-transformation (top) and after log-transformation (bottom). The horizontal line in each plot indicates the measurement when counting method was used, which simply count number of non-synonymous mutation per Mb.

FIG. 14B illustrates the biases, upper and lower limits calculated when various proportions of non-synonymous mutation were used. The first column of each figure shows the Bland Altman analysis for TMB prediction by counting method. The result using non-log-transformation value was shown in top and log-transformation in bottom. The middle circle indicates the bias (mean difference) and two solid lines around it are 95% confidence interval for the bias. The two dotted line on the top are 95% confidence intervals for the upper limit of 95% agreement and ones on the bottom are 95% confidence intervals for the lower limit of 95% agreement.

FIG. 14C sets forth plots which show the overall accuracy and kappa score for classifications of three different TMB subtypes by ecTMB when different proportions of non-synonymous mutation were used. The horizontal dashed lines in each plot indicates the measurements when counting method was used. Kappa score refers to Cohen's kappa coefficient. It is a statistic to measure the agreement between two classifiers.

$$Kappa score = \frac{{p}_{o} - {p}_{e}}{1 - {p}_{e}} ,$$

where p₀ is observed agreement among classifiers and pₑ is hypothetical probability of agreement by chance.

FIG. 15A provides scatter plots which show WES-based standard TMB plotted against predicted panel-based TMBs for each cancer types and each panel. Two methods were used for panel-based TMB predictions, including counting method (in cyan) and ecTMB method (in red). Their linear regression lines against WES-based TMB and performance measurements (correlation coefficient, MAE and RMSE) were plotted for each method in each scatter plot.

FIG. 15B provides a series of Bland Altman analysis results for the counting method (cyan) and ecTMB method (red) against WES-based TMB. The middle circle indicates the bias (mean difference) and two solid lines around it are 95% confidence interval for the bias. The two dotted line on the top are 95% confidence intervals for the upper limit of 95% agreement and ones on the bottom are 95% confidence intervals for the lower limit of 95% agreement.

FIGS. 16A, 16B, and 16C provide distribution plots of log transformed TMB for colorectal (FIG. 16A), endometrial (FIG. 16B), and stomach cancers (FIG. 16B). Three subtypes were determined by Gaussian Mixture Model classification and labeled with black (TMB-Low), orange (TMB-High) and blue (TMB-Extreme) in allClass bar. MSI status for each subject was shown with green (MSS) and red (MSI-H) in msi bar. Non-synonymous mutation existence (occurrence >1) in POLE or dMMR pathway genes, including MLH1, MLH3, MSH2, MSH3, MSH6, PMS1, PMS2 are shown in blue and wild type are shown in yellow.

FIG. 17 provides distribution plots of TMB for each cancer type in log scale (left panel). A heatmap of distribution of log-transformed TMB is provided in the right panel. K-means clustering method was used to generate five clusters, which is shown on the left side.

FIGS. 18A, 18B, 18C, 18D, and 18E provide the distributions of log-transformed TMB for each cancer: group 1 (A), group 2 (B), group 3 (C), group 4 (D) and group 5 (E). The distribution of log-transformed TMB for each individual cancer in each group is shown on the left.

FIGS. 19A, 19B, 19C, 19D, and 19E set forth landscape of mutations in MLH1 (FIG. A), PMS1 (FIG. B), MSH2 (FIG. C), MSH6 (FIG. D) and PMS2 (FIG. E) compared between TMB-high (top) and aggregated TMB-extreme and TMB-low group (bottom). The incidence of a mutation is illustrated in y axis. Various types of mutations are labeled in blue (Frame_Shift_del), purple (Frame_Shift_Ins), green (Missense_Mutation), orange (Nonsenese_mutation) and yellow (Splice_Site).

FIGS. 20A, 20B, and 20C provide plots showing the mean of predicted panel-based TMB and standard WES-based TMB for each sample as plotted against its difference (i.e. plots of Bland-Altman analysis, which plots the mean difference in x axis and mean of two measure of a same object in y. The Bland-Altman analysis is described above. The dashed line in the center of purple area indicates the bias (mean difference) and the purple area indicates the 95% confidence interval of bias. The green area shows the upper limits and its 95% confidence interval and the red area shows the lower limits and its 95% confidence interval. The Bland Altman analyses were done for FoundationOne (A), MSK-IMPACT (B), and TST170 panels. The predictions made by counting method were shown on top and ecTMB on bottom.

FIG. 21 provides scatter plots comparing WES-based standard TMB with TMB predicted by counting non-synonymous mutations after removing COSMIC variants (blue) or adding synonymous mutation (yellow).

FIG. 22 provides scatter plots which show WES-based standard TMB plotted against predicted panel-based TMBs for each cancer type and panel combination. Two methods were used for panel-based TMB predictions, including the counting method (in cyan) and ecTMB (in red). Their linear regression lines against WES-based TMB and performance measurements (correlation coefficient, MAE and RMSE) were plotted for each method in each scatter plot. Bland Altman analysis results for counting method (cyan) and ecTMB (red) against WES-based TMB are shown. The middle circle indicates the bias (mean difference) and two solid lines around it are 95% confidence interval for the bias. The two dotted line on the top are 95% confidence intervals for the upper limit of 95% agreement and ones on the bottom are 95% confidence intervals for the lower limit of 95% agreement.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “biological sample,” “tissue sample,” “specimen” or the like refer to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the term “dMMR” stands for deficient mismatch repair. MSI-H/dMMR can occur when a cell is unable to repair mistakes made during the division process.

As used herein, the term “immunotherapy” refers to the treatment of a subject afflicted with, or at risk of contracting or suffering a recurrence of, a disease by a method comprising inducing, enhancing, suppressing or otherwise modifying the immune system or an immune response. In certain embodiments, the immunotherapy comprises administering an antibody to a subject. In other embodiments, the immunotherapy comprises administering a small molecule to a subject. In other embodiments, the immunotherapy comprises administering a cytokine or an analog, variant, or fragment thereof.

As used herein, the term “Indel” refers to an insertion or deletion of bases in the genome of an organism. It is classified among small genetic variations, measuring from 1 to 10 000 base pairs in length.

As used herein, the term “MSI-H” stands for microsatellite instability-high. In general, this describes cancer cells that have a greater than normal number of genetic markers called microsatellites. Microsatellites are short, repeated, sequences of DNA. Cancer cells that have large numbers of microsatellites may have defects in the ability to correct mistakes that occur when DNA is copied in the cell. Microsatellite instability is found most often in colorectal cancer, other types of gastrointestinal cancer, and endometrial cancer. It may also be found in cancers of the breast, prostate, bladder, and thyroid.

As used herein, the terms “non-synonymous mutation” or “non-synonymous substitution” refer to a nucleotide mutation that alters the amino acid sequence of a protein. Non-synonymous substitutions differ from synonymous substitutions, which do not alter amino acid sequences and are (sometimes) silent mutations. As non-synonymous substitutions result in a biological change in the organism. Non-synonymous mutations have a much greater effect on an individual than a synonymous mutation. An insertion or deletion of a single nucleotide in the sequence during transcription is just one possible source of a non-synonymous mutation. It is believed, however, that the majority of the non-synonymous mutations are caused by substitutions of a single nucleotide. It is believed that a non-synonymous mutation with a single nucleotide substitution will alter amino acid sequences through either a substitution of a different amino acid called missense mutation or replacing original amino acid with a stop codon called nonsense mutation. The nonsense mutation will cause early termination of RNA transcription.

As used herein, the terms “panel” or “cancer panel” refer to a method of sequencing a subset of targeted cancer genes. In some embodiments, the panel comprises sequencing at least about 15, at least about 20, at least about 25, at least about 30, at least about 35, at least about 40, at least about 45, or at least about 50 targeted cancer genes.

As used herein, the term “POLE gene” refers to a gene which encodes the catalytic subunit of DNA polymerase epsilon. The enzyme is involved in DNA repair and chromosomal DNA replication. Mutations in this gene have been associated with an increased risk for autosomal dominant colonic adenomatous polyps and with colorectal cancer.

As used herein, the term “programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

As used herein, the term “programmed Death Ligand-1” (PD-L1) refers to one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

As used herein, the terms “sequence data” or “sequencing data” refer to any sequence information on nucleic acid molecules known to the skilled person. The sequence data can include information on DNA or RNA sequences, modified nucleic acids, single strand or duplex sequences, or alternatively amino acid sequences, which have to converted into nucleic acid sequences. The sequence data may additionally comprise information on the sequencing device, date of acquisition, read length, direction of sequencing, origin of the sequenced entity, neighboring sequences or reads, presence of repeats or any other suitable parameter known to the person skilled in the art. The sequence data may be presented in any suitable format, archive, coding or document known to the person skilled in the art. In some embodiments, sequencing data may be training data (e.g. from a cohort of patients having a specific type of cancer) or test data (e.g. from a “new” tumor sample from a subject).

As used herein, the terms “single nucleotide variant” or “SNV” refer to variations in a single nucleotide without any limitations of frequency and may arise in somatic cells.

As used herein, the term “somatic mutation” as used herein refers to an acquired alteration in DNA that occurs after conception. Somatic mutations can occur in any of the cells of the body except the germ cells (sperm and egg) and therefore are not passed on to children. These alterations can, but do not always, cause cancer or other diseases. The term “germline mutation” refers to a gene change in a body's reproductive cell (egg or sperm) that becomes incorporated into the DNA of every cell in the body of the offspring. Germline mutations are passed on from parents to offspring. Also called a “hereditary mutation.” In the analysis of TMB, germline mutations are considered as a “baseline,” and are subtracted from the number of mutations found in the tumor biopsy to determine the TMB within the tumor. As germline mutations are found in every cell in the body, their presence can be determined via less invasive sample collections than tumor biopsies, such as blood or saliva. Germline mutations can increase the risk of developing certain cancers and can play a role in the response to chemotherapy.

As used herein, the term “subject” includes any human or nonhuman animal, e.g. a human patient. In some embodiments, the subject has a tumor, has cancer or is suspected of having cancer.

As used herein, the terms “synonymous mutation” or “synonymous substitution” refer to the evolutionary substitution of one base for another in an exon of a gene coding for a protein, such that the produced amino acid sequence is not modified. Said another way, synonymous mutations are point mutations, meaning they are just a miscopied DNA nucleotide that only changes one base pair in the RNA copy of the DNA. In some embodiments, a synonymous mutation is a change in the DNA sequence that codes for amino acids in a protein sequence but does not change the encoded amino acid. Due to the redundancy of the genetic code (multiple codons code for the same amino acid), these changes usually occur in the third position of a codon. For example, GGT, GGA, GGC, and GGG all code for glycine. Any change in the third position of the codon (e.g. A->G), will result in the same amino acid being incorporated in the protein sequence at that position.

As used herein, a “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. Tumor mutational burden (TMB) can also be used interchangeably with “tumor mutational load,” “tumor mutational burden,” or “tumor mutation load.” In some embodiments, a TMB status can be a numerical value or a relative value, e.g., extreme, high, or low; within the highest fractile, or within the top tertile, of a reference set.

Overview

Among new biomarkers predictive for a response to immunotherapy, mutational load or tumor mutational burden has been shown to correlate with the response to immunotherapy treatment. Tumor mutational burden presents a quantitative measure of the total number of somatic non-synonymous mutations per coding area of a tumor genome. Unlike most cancer biomarkers for immunotherapies, which are specific to certain immune proteins expressed by the tumor, TMB is derived solely from mutations. It has been hypothesized that tumors with a higher mutation burden are more likely to express neoantigens and to induce a more robust immune response in the presence of immune checkpoint inhibitors. Indeed, it has been found that some tumors with a higher number of somatic mutations may be more susceptible to an immune response, and it is thus important to determine those tumors having a relatively higher tumor mutational burden such that appropriate therapeutics may be identified and administered. For example, a patient having a cancer subtype classified as “extreme TMB” may be more responsive to a particular therapeutic treatment (e.g. with a checkpoint inhibitor) than a patient having a cancer subtype classified as “high TMB” or “low TMB.” Thus, tumor mutational burden may serve as a robust biomarker for predicting efficacy of immunotherapy. Given the inconsistencies noted above with regard to the calculation of tumor mutational burden, Applicant has developed an improved method of calculating tumor mutational burden that utilizes both identified non-synonymous mutations and synonymous mutations, the new method advantageously removing driver gene effects.

The present disclosure provides systems and methods of classifying and/or identifying a cancer subtype. In some embodiments, the present disclosure provides methods of predicting tumor mutational burden and/or identifying a cancer subtype based on the predicted tumor mutational burden for a test sample. The present disclosure is based, at least in part, on the discovery that determining the level of somatic mutations (e.g. synonymous mutations and/or non-synonymous mutations) in tumor tissue samples obtained from a subject, predicting tumor mutational burden, and/or classifying cancer subtypes can be used as a biomarker (e.g., a predictive biomarker) in the treatment of a subject suffering from cancer, in the treatment of a subject suspect as having cancer, for diagnosing a subject suffering from cancer or suspected of having cancer, and/or for determining whether a subject having a cancer is likely to respond to treatment with an anti-cancer therapy (e.g. a therapy including an immune checkpoint inhibitor, such as an anti-PD-L1 antibody).

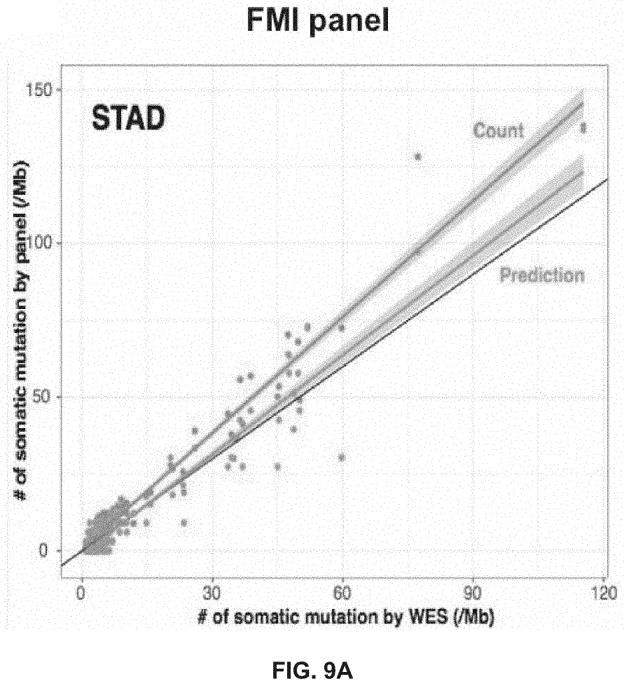

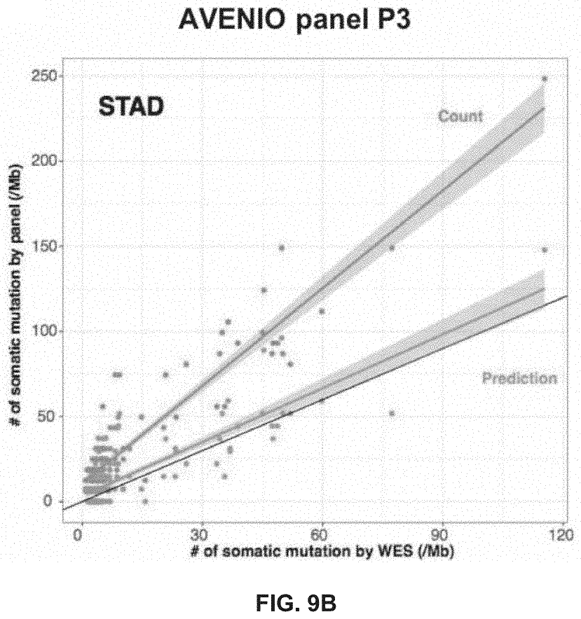

The present disclosure also provides methods of enhancing the prediction of a tumor mutational burden by using both synonymous and non-synonymous somatic mutations in the computation method. It is believed that by increasing the number of mutations in the computation of the tumor mutational burden, a comparatively more consistent tumor mutational burden may be derived, especially for targeted-panel sequencing (compare FIGS. 9A and 9B). The current standard for TMB measurement requires counting the number of non-synonymous somatic mutations in whole-exome sequencing of a tumor sample with a matched normal sample (referred to herein as the “counting method”). Clinical diagnostics, however, based on sequencing technologies still heavily relies on targeted panel sequencing. Thus, the key challenge is the inconsistency of a panel-based TMB measurement as compared to that of WES-based using the counting method. As noted above, it is believed that a panel-based TMB may overestimate TMB due to panel's enrichment of driver mutations and mutation hot spots when the counting method is applied. Two targeted panel examples, shown in FIGS. 9A (FMI panel) and 9B (AVENIO panel), illustrate that counting method over-estimates the TMB compared to the current standard TMB measurement (in x-axis) by the counting method (in blue). The methods proposed herein provide for TMB estimations for panels (in red) which are superior to the counting method, since the presently disclosed methods are comparatively more consistent than TMB estimation by the counting method. It is also believed that driver mutation effects may be systematically removed by using both synonymous and non-synonymous somatic mutations in the tumor mutational burden computation method.

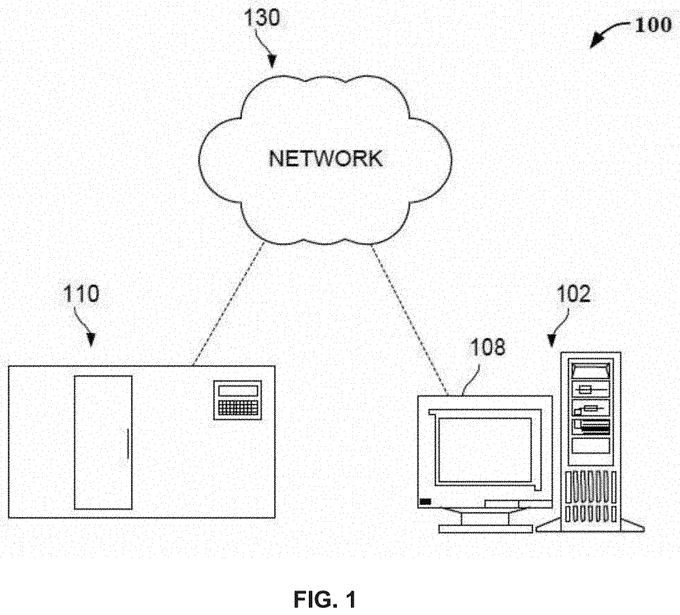

FIG. 1 sets forth a system 100 including a sequencing device 110 communicatively coupled to a processing subsystem 102. The sequencing device 110 can be coupled to the processing subsystem 102 either directly (e.g., through one or more communication cables) or through one or more wired and/or wireless networks 130. In some embodiments, the processing subsystem 102 may be included in or integrated with the sequencing device 110. In some embodiments, the system 100 may include software to command the sequencing device 110 to perform certain operations using certain user configurable parameters, and to send resulting sequencing data acquired to the processing subsystem 102 or a storage subsystem (e.g. a local storage subsystem or a networked storage device). In some embodiments, either the processing subsystem 102 or the sequencing device 110 may be coupled to a network 130. In some embodiments, a storage device is coupled to the network 130 for storage or retrieval of sequence data, patient information, and/or other tissue data. The processing subsystem 102 may include a display 108 and one or more input devices (not illustrated) for receiving commands from a user or operator (e.g. a technician or a geneticist). In some embodiments, a user interface is rendered by processing subsystem 102 and is provided on display 108 to (i) to retrieve data from a sequencing device; (iii) to retrieve patient information and/or other clinical information from a database or storage system 240, such as one available through a network; (iii) or to perform further processing operations utilizing the sequencing data.

Processing subsystem 102 can include a single processor, which can have one or more cores, or multiple processors, each having one or more cores. In some embodiments, processing subsystem 102 can include one or more general-purpose processors (e.g., CPUs), special-purpose processors such as graphics processors (GPUs), digital signal processors, or any combination of these and other types of processors. In some embodiments, some or all processors in processing subsystem can be implemented using customized circuits, such as application specific integrated circuits (ASICs) or field programmable gate arrays (FPGAs). In some embodiments, such integrated circuits execute instructions that are stored on the circuit itself. In other embodiments, processing subsystem 102 can retrieve and execute instructions stored in storage subsystem and/or one or more memories, and the instructions may be executed by processing subsystem 102. By way of example, processing subsystem 102 can execute instructions to receive and process sequencing data stored within a local or networked storage system.

A storage subsystem 240 can include various memory units such as a system memory, a read-only memory (ROM), and a permanent storage device. A ROM can store static data and instructions that are needed by processing subsystem and other modules of system. The permanent storage device can be a read-and-write memory device. This permanent storage device can be a non-volatile memory unit that stores instructions and data even when system is powered down. In some embodiments, a mass-storage device (such as a magnetic or optical disk or flash memory) can be used as a permanent storage device. Other embodiments can use a removable storage device (e.g., a flash drive) as a permanent storage device. The system memory can be a read-and-write memory device or a volatile read-and-write memory, such as dynamic random-access memory. The system memory can store some or all of the instructions and data that the processor needs at runtime. Storage subsystem can include any combination of non-transitory computer readable storage media including semiconductor memory chips of various types (DRAM, SRAM, SDRAM, flash memory, programmable read-only memory) and so on.

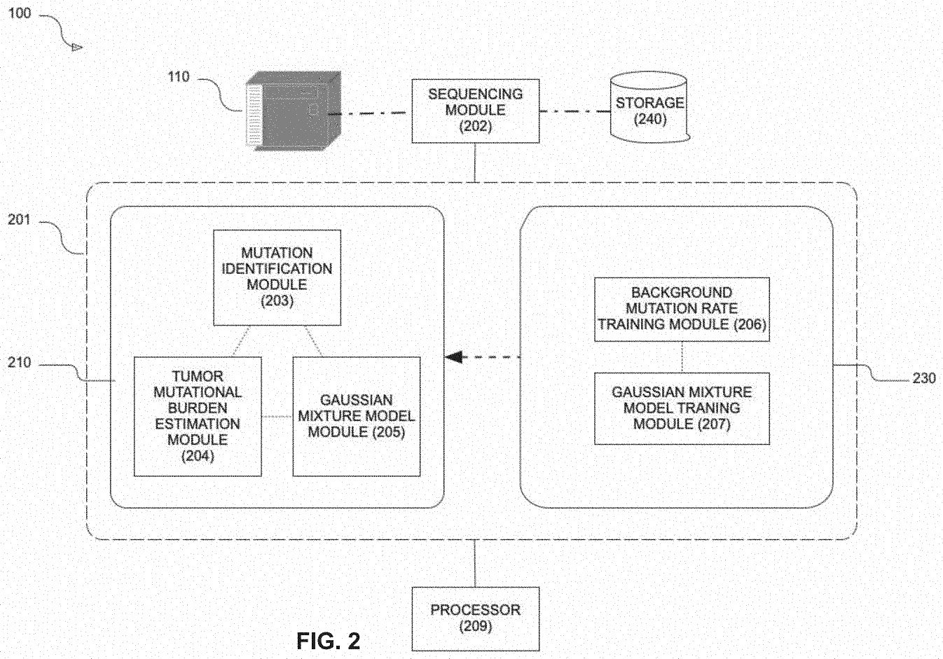

FIG. 2 provides an overview of the various modules utilized within the presently disclosed system. In some embodiments, the system employs a computer device or computer-implemented method having one or more processors 209 and one or more memories 201, the one or more memories 201 storing non-transitory computer-readable instructions for execution by the one or more processors to cause the one or more processors 209 to execute instructions (or stored data) in one or more modules (e.g. modules 202 through 207). In some embodiments, the system includes a training module 230 and a testing module 210, both of which will be described herein.

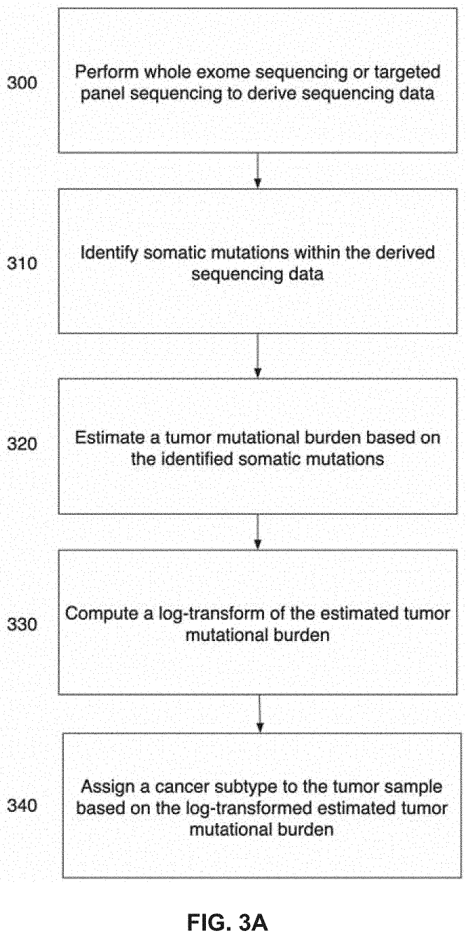

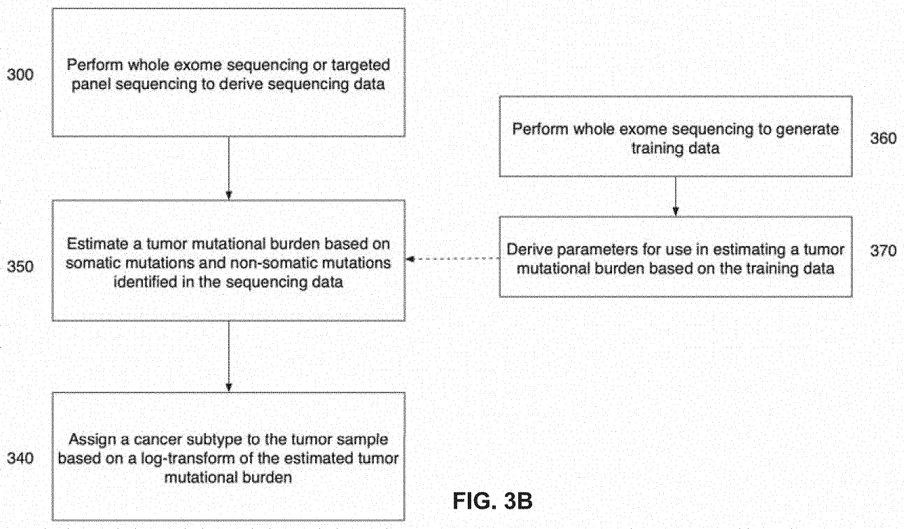

With reference to FIGS. 2, 3A, and 3B, the present disclosure provides a system for classifying a tumor sample (such as one derived from a human patient) comprising: a sequencing module 202 to generate sequencing data (step 310); a mutation identification module 203 to identify somatic mutations within acquired sequencing data (step 3210); a tumor mutational burden estimation module 204 to estimate a tumor mutational burden based on identified somatic mutations (step 320) and to compute a log-transform of the estimated tumor mutational burden (step 330); and a Gaussian mixture model module 205 to assign a cancer subtype to the tumor sample based on the log-transformed estimated tumor mutational burden (step 340). In some embodiments, modules 203, 204, and 205 are part of a testing module 210 whereby a biological sample, e.g. a tumor sample derived from a patient diagnosed with cancer or suspected of having cancer, is classified.

Again, with reference to reference to FIGS. 2, 3A, and 3B, the present disclosure also provides for a training module 230. In some embodiments, the training module is part of system 100. In other embodiments, the training module is part of a different system, but where training data derived from training using the training module 230 is supplied to testing module 210 such that a tumor sample may be classified based on training data (e.g. parameters derived from training). In some embodiments, the training module 230 may comprise one or both of a background mutation rate training module 206 or a gaussian mixture model training module 207. In some embodiments, a background mutation rate training module 206 such that parameters for use in estimating the tumor mutational burden (step 370) may be derived. As such, in some embodiments, and with reference to FIG. 3B, the system may use the background mutation rate training module 206 is utilized to derive one or more parameters for use in estimating a tumor mutational burden based on input training data (e.g. input training data derived from whole exome sequencing) (see step 360), where the parameters are ultimately used within a maximum likelihood estimation process for deriving the estimated tumor mutational burden (step 370). In some embodiments, the system may further include a Gaussian mixture model training module 208 such that parameters for used in modeling log-transformed TMBs may be modeled within a Gaussian mixture model. The skilled artisan will also appreciate that additional modules may be incorporated into the workflow, and for use with either the training module 230 or the testing module 210. In some embodiments, the training module 230 may share some of modules 203, 204, and 205 with the testing module 210.

Sequencing Module

In some embodiments, a nucleic acid sample (DNA, cDNA, mRNA, exoRNA, ctDNA, and cfDNA) derived from a biological sample is sequenced (step 300). In some embodiments, a nucleic acid sample may be isolated from any type of suitable biological specimen or sample (e.g., a test sample). In the context of cancer, non-limiting examples of biological samples include cancerous tumors, benign tumors, metastatic tumors, lymph nodes, blood, or any combination thereof. In some embodiments, the biological sample is a tumor tissue biopsy, e.g., a formalin-fixed, paraffin-embedded (FFPE) tumor tissue or a fresh-frozen tumor tissue or the like. In some embodiments, the biological sample is a liquid biopsy that, in some embodiments, comprises one or more of blood, serum, plasma, circulating tumor cells, exoRNA, ctDNA, and cfDNA. As used herein, the term “blood” encompasses whole blood or any fractions of blood, such as serum and plasma as conventionally defined, for example.

Advances in sequencing technologies allow for evaluation of the tumor's genomic mutation landscape and/or the generation of sequencing data for downstream analysis. Any sequencing methods known to those of skill in the art can be used to sequence nucleic acids from the biological sample. For example, methods of sequencing a sample are described in PCT Publication Nos. WO/2017/123316 and WO/2017/181134, the disclosures of which are hereby incorporated by reference herein in their entireties.

In some embodiments, sequencing methods include PCR or qPCR methods, Sanger sequencing and dye-terminator sequencing, as well as next-generation sequencing technologies (such as genomic profiling and exome sequencing) including pyrosequencing, nanopore sequencing, micropore-based sequencing, nanoball sequencing, MPSS, SOLiD, Illumina, Ion Torrent, Starlite, SMRT, tSMS, sequencing by synthesis, sequencing by ligation, mass spectrometry sequencing, polymerase sequencing, RNA polymerase (RNAP) sequencing, microscopy-based sequencing, microfluidic Sanger sequencing, microscopy-based sequencing, RNAP sequencing, tunneling currents DNA sequencing, and in vitro virus sequencing. Such methods are described within PCT Publication Nos. WO/2014/144478, WO/2015/058093, WO/2014/106076 and WO/2013/068528, the disclosures of which are hereby incorporated herein by reference in their entireties.

Sequencing by synthesis is defined as any sequencing method which monitors the generation of side products upon incorporation of a specific deoxynucleoside-triphosphate during the sequencing reaction (Hyman, 1988, Anal. Biochem. 174:423-436; Rhonaghi et al., 1998, Science 281:363-365). In some embodiments, sequencing by synthesis reaction utilizes a pyrophosphate sequencing method. In this case, generation of a pyrophosphate during nucleotide incorporation is monitored by an enzymatic cascade which results in the generation of a chemo-luminescent signal. In some embodiments, a sequencing by synthesis reaction can alternatively be based on a terminator dye type of sequencing reaction. In this case, the incorporated dye deoxynucleotriphosphates (ddNTPs) building blocks comprise a detectable label, which is preferably a fluorescent label that prevents further extension of the nascent DNA strand. The label is then removed and detected upon incorporation of the ddNTP building block into the template/primer extension hybrid for example by using a DNA polymerase comprising a 3′-5′ exonuclease or proofreading activity. In some embodiments, sequencing is performed using a next-generation sequencing method such as that provided by Illumina, Inc. (the “Illumina Sequencing Method”). It is believed that the process simultaneously identifies DNA bases while incorporating them into a nucleic acid chain. Each base emits a unique fluorescent signal as it is added to the growing strand, which is used to determine the order of the DNA sequence.

Nanopore sequencing of a polynucleotide, e.g. DNA or RNA, may be achieved by strand sequencing and/or exosequencing of the polynucleotide sequence. In some embodiments, strand sequencing comprises methods whereby nucleotide bases of a sample polynucleotide strand are determined directly as the nucleotides of the polynucleotide template are threaded through the nanopore. In some embodiments, nanopore-based nucleotide acid sequencing uses a mixture of four nucleotide analogs that can be incorporated by an enzyme into a growing strand. In some embodiments, a polynucleotide can be sequenced by threading it through a microscopic pore in a membrane. In some embodiments, bases can be identified by the way they affect ions flowing through the pore from one side of the membrane to the other. In some embodiments, one protein molecule can “unzip” a DNA helix into two strands. A second protein can create a pore in the membrane and hold an “adapter” molecule. A flow of ions through the pore can create a current, whereby each base can block the flow of ions to a different degree, altering the current. The adapter molecule can keep bases in place long enough for them to be identified electronically (see PCT Publication No. WO/2018/034745, and United States Patent Application Publication Nos. 2018/0044725 and 2018/0201992, the disclosures of which are hereby incorporated by reference herein in their entireties).

In some embodiments, whole exome sequencing is performed (step 300). Exomes are the part of the genome formed by exons, or coding regions, which when transcribed and translated become expressed into proteins. Exomes compose only about 2% of the whole genome. Because the whole genome is so much larger, exomes are able to be sequenced at a much greater depth (number of times a given nucleotide is sequenced) for lower cost. This greater depth is believed to provide more confidence in low frequency alterations.

Sequencing depth can become even greater for lower cost by using a targeted or “hot-spot” sequencing panel, which has a select number of specific genes, or coding regions within genes that are known to harbor mutations that contribute to pathogenesis of disease (e.g. a type of cancer) and may include clinically-actionable genes of interest. As such, in some embodiments, targeted sequencing is performed, such as a targeted panel for a specific disease, disorder, or cancer (step 300). In some embodiments, genomic (or gene) profiling methods can involve panels of a predetermined set of genes, e.g., 150-500 genes, and in some instances the genomic alterations evaluated in the panel of genes are correlated with total somatic. In some embodiments, genomic profiling involves a panel of a predefined set of genes comprising as few as five genes or as many as 1000 genes, about 25 genes to about 750 genes, about 100 genes to about 800 genes, about 150 genes to about 500 genes, about 200 genes to about 400 genes, about 250 genes to about 350 genes. In one embodiment, the genomic profile comprises at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, or at least 400 genes. In another embodiment, the genomic profile comprises at least 325 genes. The development of targeted custom panels is described in US Publication No. 2009/0246788, the disclosure of which is hereby incorporated by reference herein in its entirety.

Examples of panels include FoundationOne CDx and the Memorial Sloan Kettering-Integrated Mutation Profiling of Actionable Cancer Targets (MSK-IMPACT) targeted sequencing panel, which targets 468 individual cancer-related genes, thereby covering 1.5 Mb of the human genome. Another example of a panel is the FOUNDATIONONE® assay, which is believed to be a comprehensive genomic profiling assay for solid tumors, including but not limited to solid tumors of the lung, colon, and breast, melanoma, and ovarian cancer. It is believed that the FOUNDATIONONE® assay uses a hybrid-capture, next-generation sequencing test to identify genomic alterations (base substitutions, insertions and deletions, copy number alterations, and rearrangements) and select genomic signatures (e.g., TMB and microsatellite instability). The assay covers 322 unique genes, including the entire coding region of 315 cancer-related genes, and selected introns from 28 genes.

In some embodiments, the sequencing data derived after sequencing the input biological sample (or nucleic acid sample derived from the biological sample) may be stored in storage subsystem 240 for later retrieval. In some embodiments, the sequencing data acquired may be supplied to a testing module 210, such as to a mutation identification module 203. Alternatively, stored sequencing data may be retrieved and may be supplied to the testing module 230 such that training data may be generated.

Mutation Identification Module

Following sequencing (step 300), the sequencing data may be analyzed such that somatic mutations may be identified within the sequencing data (step 310). In some embodiments, sequencing data is retrieved from the storage system 240. In some embodiments, the sequencing data comprises test data, i.e. sequencing data derived from a biological sample derived from a patient. In other embodiments, the sequencing data is training data, i.e. sequencing data derived from a publicly available database and which includes sequencing data of multiple patients having the same type of disease, e.g. the same type of cancer.

In some embodiments, MuTect is used to detect mutations within sequencing data (see https://software.broadinstitute.org/cancer/cga/mutect; and see also U.S. Patent Publication No. 2015/0178445, the disclosures of which are hereby incorporated by reference herein in their entireties). For example, MuTect can take as input paired tumor and normal next generation sequencing data and, after removing low quality reads, determines if there is evidence for a variant beyond the expected random sequencing errors (variant detection will be discussed in more detail below). Candidate variant sites are then passed through, for example, one or more filters to remove sequencing and alignment artifacts. Next, a Panel of Normals can be used to screen out remaining false positives caused by rare error modes only detectable using more samples. Finally, the somatic or germline status of passing variants is determined using the matched normal.

In some embodiments, MuTect can take as input sequence data from matched tumor and normal DNA after alignment of the reads to a reference genome and preprocessing steps which include, for example, marking of duplicate reads, recalibration of base quality scores and local realignment. The method operates on each genomic locus independently and consists of four key steps: (i) Removal of low-quality sequence data (based on known methods); (ii) variant detection in the tumor using a Bayesian classifier; (iii) filtering to remove false positives resulting from correlated sequencing artifacts that are not captured by the error model; and (iv) designation of the variants as somatic or germline by a second Bayesian classifier.

In some embodiments, statistical analysis predicts a somatic mutation by using two Bayesian classifiers—the first aims to detect whether the tumor is non-reference at a given site and, for those sites that are found as non-reference, the second classifier makes sure the normal does not carry the variant allele. In practice the classification is performed by calculating a LOD score (log odds) and comparing it to a cutoff determined by the log ratio of prior probabilities of the considered events.

As an alternative to MuTect, other somatic variant callers include MuSE, VarScan, VarDict, NeuSomatic, SomaticSeq, SEURAT and STRELKA. In some embodiments, mutations within sequencing data may be identified using any of the systems and methods disclosed within U.S. Publication Nos. 2017/0132359 and 2017/0362659, the disclosures of which are hereby incorporated by reference herein in their entireties.

In some embodiments, the identification of somatic mutations comprises identifying both non-synonymous and synonymous mutations. In other embodiments, the identification of somatic mutations comprises identifying only synonymous mutations. In some embodiments, each mutation may be annotated by a variant effect predictor, which can predict the effect of the mutations, including whether the mutation is a synonymous mutation or a non-synonymous mutation. (see McLaren et al., “The Ensembl Varient Effect Predictor,” Genome Biology 2016, 17:122, the disclosure of which is hereby incorporated by reference herein in its entirety).

Once identified, the non-synonymous and synonymous mutations may be stored in storage module 240 for later retrieval and/or downstream processing.

Tumor Mutational Burden Estimation Module

Subsequently, a tumor mutational burden is estimated (step 320) based on the identified somatic mutations (from step 310). In some embodiments, the tumor mutational burden is estimated using identified non-synonymous mutations. In these embodiments, the tumor mutational burden is estimated by dividing a total number of identified non-synonymous mutations by a pre-determined genome size, i.e. the total number of mutations identified in a sample is divided by the number of bases sequenced in sample. As an example, for a whole-exome panel, the target region may be approximately 50 Mb, and a sample with about 500 somatic mutations identified may have an estimated TMB of 10 mutations/Mb. The tumor mutational burden estimated in this manner, and based solely on non-synonymous mutations, may then be further processed, i.e. the log-transform taken, and then the log-transformed data supplied to the gaussian mixture model module 205.

In some embodiments, tumor mutational burden is estimated using identified non-synonymous mutations and identified synonymous mutations (step 350). In some embodiments, the tumor mutational burden is estimated by performing a maximum likelihood estimation using the identified non-synonymous and synonymous mutations and a plurality of pre-determined mutation rate parameters. The maximum likelihood estimation is a method that determines values for the parameters of a model. In some embodiments, the parameter values are found such that they maximize the likelihood that the process described by the model produced the data that were actually observed.

For example, assume a mutation of gene A follows simply Poisson distribution with mean λ (0<λ<10). The likelihood function of this statistical model is

$$ℒ ( λ , X ) = {Π}_{1}^{S} \frac{{λ}^{{x}_{i}}}{{x}_{i} !} .$$

The observed number of mutations in gene A (X) for samples S={1,2,3 . . . } are X={5, 2, 4, . . . }. Parameter λ can be estimated using maximum likelihood method by iteratively denoting λ as a number within (0,10) until λ can maximize likelihood function

$$ℒ ( λ , X ) = {Π}_{1}^{S} \frac{{λ}^{{x}_{i}}}{{x}_{i} !} .$$

In some embodiments, using pre-defined parameters (described herein) learned from training (such as using the background mutation training module 206), each gene is modeled as an independent zero-inflated Poisson process for a given new sample s′. Then, the Maximum Likelihood estimation (MLE) is used to estimate b_{s′} (the sample mutation rate), by maximizing formula [1] using pre-defined parameters and observed mutation counts of each gene. In this step, n stands for number of genes, k is number of genes of n whose observed mutation is 0, and Y_{g}={y₁, y₂, . . . , x_{g}} are synonymous mutation counts (or part of non-synonymous mutation counts) in sample s′. In some embodiments, the parameters learned from training (i.e. learned from training using the background mutation rate training module 206) include α_{g}′, p_{g} and E_{g}, such as defined herein.

$$P ( {Y}_{g} \vert {b}_{{s}^{′}} ) = {{[ {p}_{g} + ( 1 - {p}_{g} ) ]}^{k} [ ( 1 - {p}_{g} ) ]}^{( n - k )} {Π}_{j = k + 1}^{n} \frac{ {b}_{{s}^{′}} {E}_{g}^{{y}_{i}}}{{y}_{i} !} \qquad [ 1 ]$$

In some embodiments, the plurality of pre-determined mutation rate parameters comprise (i) gene-specific mutation rate factors, and (ii) context-specific mutation rates. In some embodiments, the context-specific mutation rates are selected form the group consisting of (i) tri-nucleotide context specific mutation rates; (ii) di-nucleotide context specific mutation rates, and; (iii) mutation signatures.

Multiple studies have shown that the mutation rate of different genes is associated with the location of the gene, its expression level and the function type of the gene. For example, the mutation rate is relatively higher for genes located in regions where they are replicated late during the DNA duplication process or where they do not have an open-chromatin state. The genes with very low expression level or those which belong to the olfactory receptor gene family are believed to have a higher mutation rate. These known factors can be aggregated through regression to generate Gene-specific mutation factors (a).

It has been reported that different mutagens can cause specific mutation patterns. For example, ultraviolet light exposure dominantly causes C>T mutation with extended context TC>TT or (CIT)C>(CIT)T. The mutated DNA polymerase epsilon can dominantly cause C>T mutation in extended context TCG>TTG or TCT>TAT. (see Poon et al., “Mutation signatures of carcinogen exposure: genome-wide detection and new opportunities for cancer prevention,” Genome Medicine 20146:24, the disclosure of which is hereby incorporated by reference herein in its entirety). Also, large-cohort analysis revealed many mutational signatures, which displayed as six substitution subtypes: C>A, C>G, C>T, T>A, T>C and T>G. (see, for example, https://cancer.sanger.ac.uk/cosmic/signatures, the disclosure of which is hereby incorporated by reference herein in its entirety). Some of these mutation signatures are shown to be caused by known mutagens. For example, signature 4 in COMSMIC database is shown to be caused by smoking.

In some embodiments, once the tumor mutational burden is estimated, the estimated tumor mutational burden is then transformed (i.e. a data transformation is performed), such as to make a skewed distribution less skewed (i.e. to conform data to normality or to normalize positively skewed distributions), to provided discernable patterns, or to reduce variability (i.e. to stabilize variability). In some embodiments, the transformation is a logarithmic transformation. In some embodiments, once a tumor mutational burden is estimated (step 320), such as a tumor mutational burden estimated using (i) only non-synonymous mutations, or (ii) both non-synonymous mutations and synonymous mutations, the log-transform of the estimated tumor mutational burden may then be computed (step 330). In some embodiments, the log-transform is computed by taking the log of the estimated tumor mutational burden. The log may be, by way of example only, a natural log (i.e. Log(natural) calculates the natural (Naperian, log to the base e) of a dataset), log(10) (i.e. log (base10) calculates the common (log to the base 10) logarithm of a dataset), log(2), etc. For example, a patient with TMB 10/Mb, log 10-transformed TMB will be log 10(10)=1. If log 2 transformation is used, log 2(10)≈3.32. The log-transformed data may then be supplied to the Gaussian mixture model module 205 for further downstream processing.

Gaussian Mixture Model Module

In some embodiments, the log-transformed estimated tumor mutational burden (computed using the tumor mutational burden estimation module 204 at step 330 or 350) is modeled using a Gaussian mixture model, where each K^{th} component of the Gaussian Mixture Model represents one cancer subtype.

More specifically, log-transformed tumor mutational burdens may be modeled as Gaussian Mixture Model, in which components (K) of the Gaussian Mixture Model represent cancer subtypes (see equation [2] below). A Gaussian mixture model is a probabilistic model that assumes all the data points are generated from a mixture of a finite number of Gaussian distributions with unknown parameters. One can think of mixture models as generalizing k-means clustering to incorporate information about the covariance structure of the data as well as the centers of the latent Gaussians.

$$P ( y ) = {Σ}_{k = 1}^{K} {π}_{k} N ( {μ}_{k} , {Σ}_{k} ) \qquad [ 2 ]$$

In some embodiments, an Expectation-Maximization algorithm can be used to estimate each component's parameters in the Gaussian mixture model with training data (see equation [2]). In some embodiments, the parameters for the K^{th} component include weight (πₖ), mean (μₖ), and variance (Σₖ). These parameters are used in an assignment score calculation (described below). It is believed that the main difficulty in generating Gaussian mixture models from unlabeled data is that it is one usually doesn't know which points came from which latent component. Expectation-maximization is a well-founded statistical algorithm to get around this problem by an iterative process. First, one assumes random components (randomly centered on data points, learned from k-means, or even just normally distributed around the origin) and computes for each point a probability of being generated by each component of the model. Then, one adjusts the parameters to maximize the likelihood of the data given those assignments. Repeating this process is guaranteed to always converge to a local optimum.

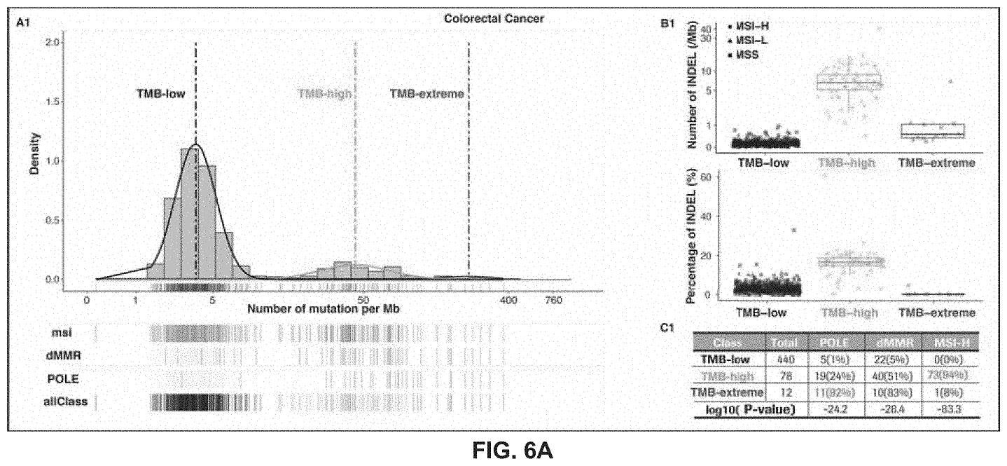

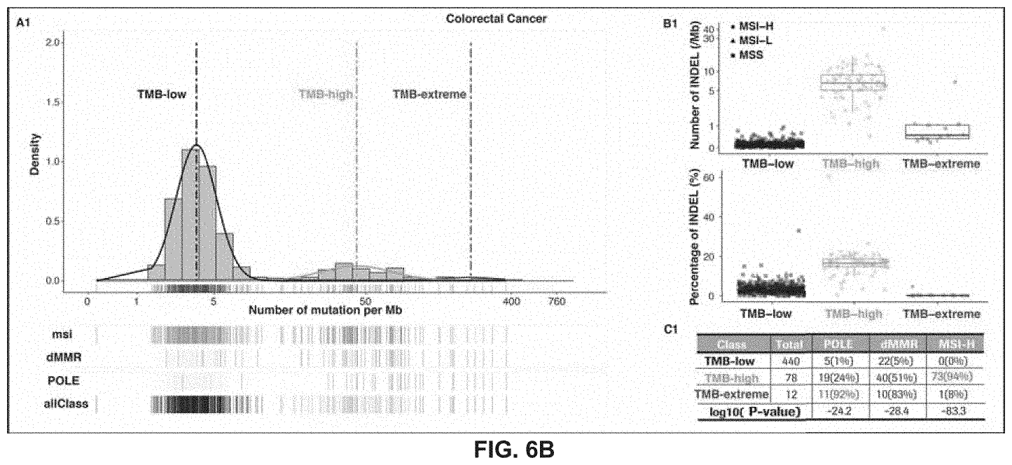

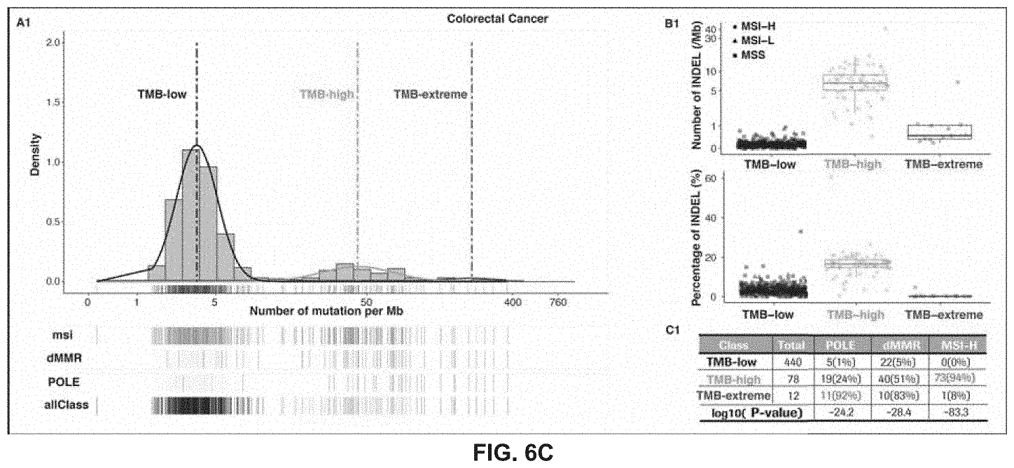

In some embodiments, modeling with the Gaussian mixture model may be used to identify cancer subtypes, such as identifying cancer subtypes using training sequencing data. In some embodiments, the cancer subtypes are “low TMB,” “high TMB,” and “extreme TMB.” A process for identifying such cancer subtypes is described in the Examples section herein (see also FIGS. 6A, 6B, and 6C).

It is believed that distinct mutation profiles and tumor-infiltrating immune cell populations were observed among these three identified cancer subtypes which were defined by log-transformed TMB according to the methods described herein. Patients of “low TMB” subtype, in some embodiments, have low mutation rate and are depleted with non-synonymous mutation in POLE gene or dMMR pathway genes. Most of the patients defined as “high TMB” have MSI-H status and a high INDEL mutation rate. Patients in the “extreme TMB” subtype are believed to have an extremely high SNV mutation rate but low INDEL mutation rate. Also, most of the “extreme TMB” patients had non-synonymous mutations in POLE gene. It was also observed that the “high TMB” and the “extreme TMB” subtypes were significantly associated with improved patient overall survival even after considering age and cancer stage compared to the “low TMB” subtype. The association of subtypes defined by log-transformed TMB and patient overall survival indicates subtype classification using log-transformed TMB can be used as a prognostic biomarker.

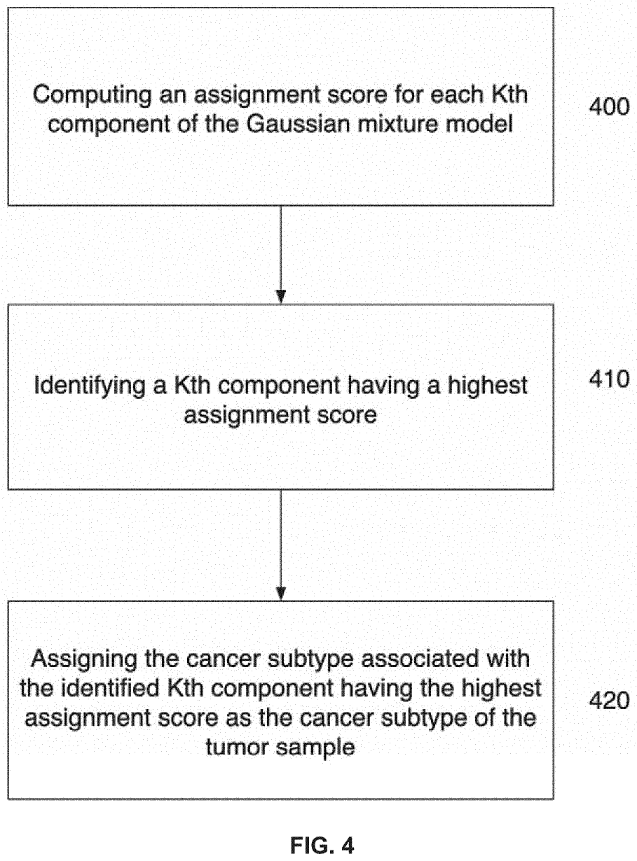

In some embodiments, and with reference to FIG. 4, modeling with the Gaussian mixture model may be used to classify cancer subtypes for a test sample (i.e. test sequencing data derived from a biological sample from a patient, e.g. a human patient diagnosed with cancer or suspected of having cancer). When classifying a cancer subtype in test sequencing data, an assignment score is computed for each K^{th} component of the Gaussian Mixture Model (step 400), as described further below. After each assignment score for each K^{th} component is computed, the K^{th} component having the highest assignment score is determined, e.g. the assignment scores may be ranked that the score having the highest ranking may be identified (step 410). In some embodiments, a cancer subtype is then assigned to a test sample, and this assignment is based on the identification of the K^{th} component having the highest assignment score (step 420), i.e. the cancer subtype associated with the K^{th} component ranked as having the highest assignment score is assigned to the test sample.

In particular, and for a given test sample's log-transformed TMB (yᵢ), the assignment score for each component (γ(b|Cₖ)) is calculated using the equation [3] using pre-defined parameters, such as those derived at step 370. In some embodiments, the assignment score for the K^{th} component equals the probability that the new log-transformed TMB belongs to the K^{th} component divided by the sum of the probability that the new log-transformed TMB belongs to each component. The test sample will be classified to the component which has the highest assignment score.

$$γ ( {y}_{i} \vert {C}_{k} ) = \frac{{π}_{k} N ( {y}_{i} \vert {μ}_{k} , {Σ}_{k} )}{{Σ}_{k = 1}^{K} {π}_{k} N ( {y}_{i} \vert {μ}_{k} , {Σ}_{k} )} \qquad [ 3 ]$$

For example, using pre-defined parameters for three components:

$$π = \{ 0.6 , 0.3 , 0.1 \} \\ μ = \{ 0.6 , 4.3 , 8 \} \\ Σ = \{ 0.1 , 1 , 3 \}$$

$$N ( x \vert μ , Σ ) = \frac{1}{Σ \sqrt{2 π}} {e}^{\frac{- {( x - μ )}^{2}}{2 {Σ}^{2}}}$$

A new sample with log-transformed TMB as 10, the assignment score for 3 components will be given as:

$$γ ( b \vert {C}_{1} ) = \frac{0.6 * N ( \vert 0.6 , 01 )}{0.6 * N ( 10 \vert 0.6 , 0.1 ) + 0.3 * N ( 10 \vert 4.3 , 1 ) + 0.1 * N ( 10 \vert 8 , 3 )} = 0 \\ γ ( b \vert {C}_{2} ) = \frac{0.3 * N ( 10 \vert 4 .3 , 1 )}{0.6 * N ( 10 \vert 0.6 , 0.1 ) + 0.3 * N ( 10 \vert 4.3 , 1 ) + 0.1 * N ( 10 \vert 8 , 3 )} = 7.1 * {10}^{- 8}$$

$$γ ( b \vert {C}_{3} ) = \frac{0.1 * N ( 10 \vert 8 , 3 )}{0.6 * N ( 10 \vert 0.6 , 0.1 ) + 0.3 * N ( 10 \vert 4.3 , 1 ) + 0.1 * N ( 10 \vert 8 , 3 )} = 0.999$$

According to this example, the assignment score for the third component is the highest, and the sample will be classified as “extreme TMB.”

Background Mutation Rate Training Module

The present disclosure also provides for methods of deriving parameters for use in estimated a tumor mutational burden (step 370), such as by using a background mutation rate training module 206. In some embodiments, the derived parameters are stored in storage system 240 for further retrieval and downstream processing, e.g. for use by the Gaussian mixture model module 205. It is believed that a method which consolidates known and unknown gene and context specific influencing factors would allow for the consistent prediction of tumor mutational burden for both targeted panel sequencing and whole exome sequencing. Such a method, it is believed, effectively removes driver gene effects by using both synonymous and partial non-synonymous mutation data, mitigating overestimation of tumor mutational burden (compare FIGS. 9A to 9B).

In some embodiments, training sequencing data is first acquired, such as whole-exome sequencing data. In some embodiments, the sequencing data acquired includes replication timing, expression level, and open-chromatin state of all protein-coding genes.

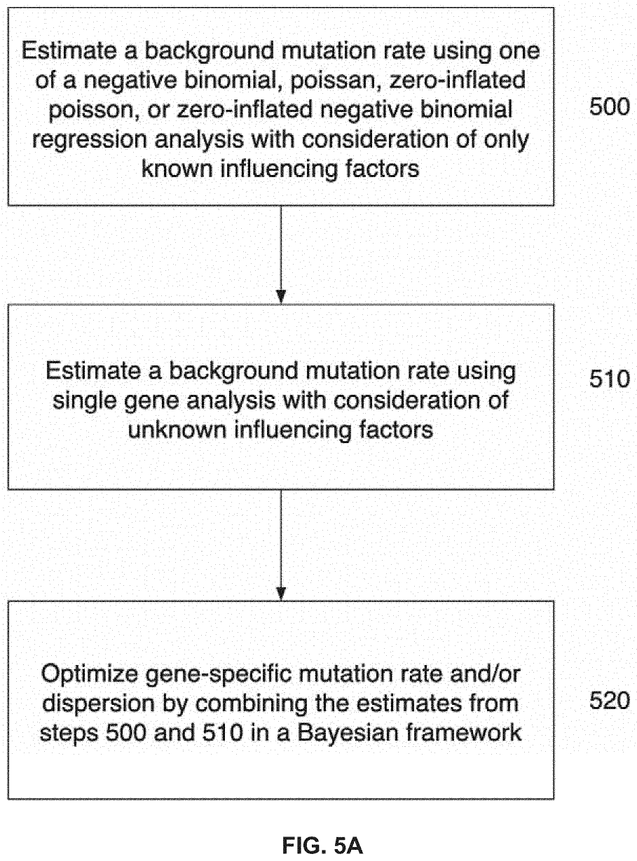

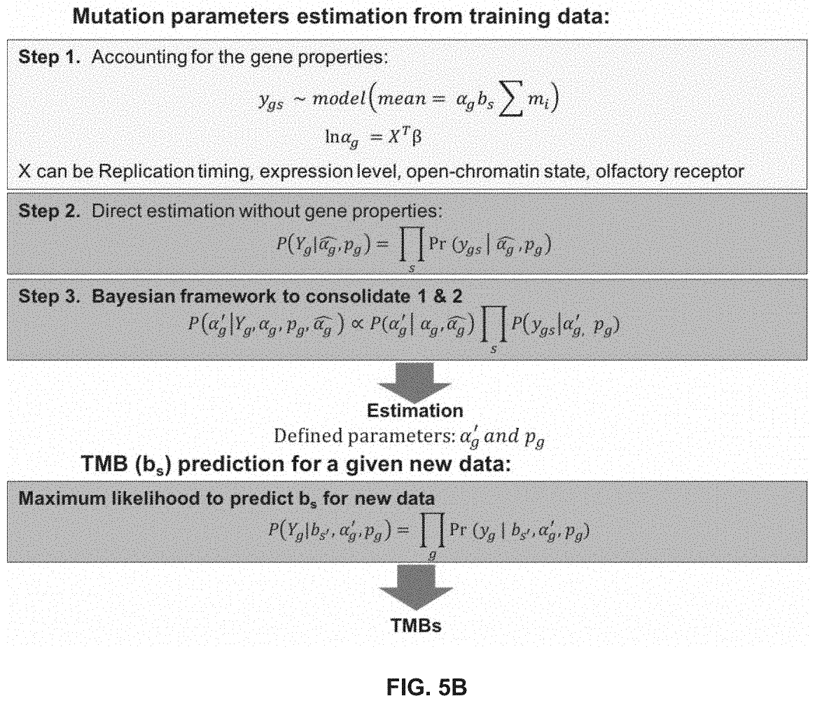

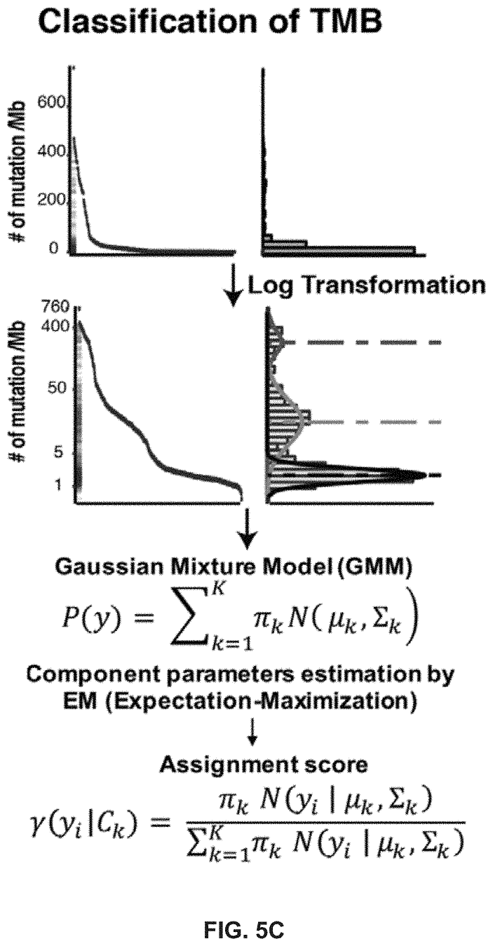

In some embodiments, and with reference to FIGS. 5A and 5B, a first set of parameters for a probability distribution of gene-specific background mutation rate for each gene of a plurality of genes, such as a first gene-specific mean (or gene-specific mean coefficient) and/or a dispersion for the probability distribution, may be determined by considering known influencing factors, such as replication timing (R), expression level (X), open-chromatin state (C), and whether gene is an olfactory receptor (O) (step 500). In some embodiments, the dispersion, if used, may be non-gene-specific and may be a genome-wide dispersion. In some embodiments, the first set of parameters may be determined using a regression technique (e.g., negative binomial repression, Poisson regression, linear regression, zero-inflated Poisson regression, zero-inflated negative binomial regression, etc.) applied to measurement results for the plurality of genes and a plurality of samples for estimating the shared effects of the known mutation influencing factors on any gene in the genome. For example, the total number of synonymous mutations in all samples for each gene may be used as one data point for determining the second set of parameters for the probability distribution.

It is believed that there are multiple factors that could influence the underlying mutation rate for modeling the synonymous mutation counts. First, the number of possible synonymous mutations is controlled by the gene's coding sequence (e.g. codons and length). More specifically, for a gene g, context-specific mutation rates for all possible bases that could mutate to synonymous mutations can be added to determine the expected number of synonymous mutations. Second, because samples from different individuals are expected to have different background mutation rates, a sample specific factor (i.e., sample mutation rate) bₛ may be used to represent the total mutation burden of a sample s. Third, several additional factors may influence the underlying mutation rate for a given gene, including replication timing (R), expression level (X), open-chromatin state (C), and whether gene is an olfactory receptor (O). Values for the replication timing, expression level, and open-chromatin state may be extracted as described in M. S. Lawrence et al., “Mutational heterogeneity in cancer and the search for new cancer-associated genes,” Nature 499, 214-8 (2013). These values can be determined by averaging across different cell lines. The values can be fixed for a given determination of mutation properties for a set of samples. These values can also be updated to be cell-line specific values for use in another determination of mutation properties.

In some embodiments, a second set of parameters for the probability distribution of gene-specific background mutation rate for each gene may be determined by considering the plurality of samples for the gene (step 510). In some embodiments, the second set of parameters may include a first gene-specific mean (or gene-specific mean coefficient) and/or a gene-specific dispersion for the probability distribution. In some embodiments, the second set of parameters may be determined by fitting the probability distribution to measured background gene mutation rates for the plurality of samples for the gene based on a number of synonymous mutations in the gene in each sample of the plurality of samples. In some embodiments, the probability distribution for each gene may include a negative binomial distribution, a Poisson distribution, or a beta binomial distribution.

In some embodiments, an optimized set of parameters for the probability distribution of gene-specific background mutation rate for each gene of the plurality of samples that best fits measurement data may be determined (step 520). The first set of parameters and the second set of parameters estimated using the techniques described above (see steps 500 and 510) may be used as prior knowledge to recursively optimize the set of parameters for the probability distribution of gene-specific background mutation rate for the gene that best fits the measurement data, using, for example, Bayesian inference or non-Bayesian inferences (e.g., classical Frequentist Prediction, likelihood-based inference, etc.). In some embodiments, the gene-specific mutation rate and/or dispersion are optimized within a Bayesian framework.

In some embodiments, the steps of deriving the parameters for use in estimating tumor mutational burden are described in further detail below:

1. Mutation Rate for Each Sample (bₛ)

The mutation rate for each sample (bₛ) is determined by the total number of mutations of the sample divided by size of evaluated genome in Mb (Megabase) unit. If only non-synonymous mutations were used, bₛ is equivalent to current standard TMB calculation.

2. Tri-Nucleotide Context-Specific Mutation Rates

Tri-nucleotide context-specific mutation rates were estimated for the training cohort. In some embodiments, the 96 possible tri-nucleotide contexts are considered (from the 6 possible types of single base substitutions—A/T->G/C, T/A->G/C, A/T->C/G, T/A->C/G, A/T->T/A, G/C->C/G—and possible nucleotides around it) plus indels. Mutations are classified as synonymous or non-synonymous based on whether they cause a change to the amino acid sequence of the translated protein. It is assumed that whether a background mutation causes a synonymous or non-synonymous effect solely depends on the nucleotide change and synonymous mutations occur according to the background mutation rate.

For each tri-nucleotide mutation context i, the number of synonymous n_{i(synonymous)} and non-synonymous n_{i(non-synonymous)} mutations observed across all tumor samples is calculated and the number of possible synonymous N_{i(synonymous)} and non-synonymous N_{i(non-synonymous)} variants in the exome is determined. For non-synonymous mutations, only genes that are less likely to be drivers, are taken into consideration to avoid skewing the background non-synonymous mutation rate; i.e. about the bottom 60% of genes ranked by the number of mutated samples in decreasing order. In some embodiments, the potential bias introduced by using a subset of genes for non-synonymous mutations is corrected by factor r, which is estimated using the method of moment, calculated as the mean of:

$${m}_{i} = \frac{{n}_{i} ( synonymous ) + {n}_{i} ( nonsynonymous )}{{N}_{i} ( synonymous ) + r \times {N}_{i} ( nonsynonymous )} \qquad [ 4 ]$$

across all mutation contexts. For mutation context i, the mutation rate mᵢ is calculated use the formula above (equation [4]). In some embodiments, when calculating indel mutation rate m_{indel}, it is assumed that all protein-coding positions can have indels, and that all indels are considered as non-synonymous.

3. Gene-Specific Mutation Rate Factor α_{g}

(3i) Regression Model Across Genes

It is assumed that the occurrence of synonymous mutations represents the background mutation rate, and that the number of synonymous mutations per gene can be modeled using the negative binomial, and Poisson regression (see within PCT Publication No. WO/2017/181134, the disclosure of which is hereby incorporated by reference herein in its entirety). In some embodiments, a zero-inflated Poisson regression is utilized. It is believed that this technique suggests that the excessive zeros can be generated by a separate process so that it can model over-dispersed data.

Multiple factors are considered that could influence the underlying mutation rate to model the synonymous mutation counts. In some embodiments, the number of possible synonymous mutations is controlled by the gene's coding sequence (e.g. codons and length). Specifically, for gene g, we get all possible bases that could mutate to synonymous mutations and sum up their context-specific mutation rate as E_{g(synonymous)}=Σ_{synonymous base} mᵢ. Second, because different individuals are expected to have different background mutation rates, a sample specific factor bₛ is used to represent total mutation burden of sample s. In some embodiments, bₛ is the total number of mutations divided by the number of bases sequenced in the sample. Third, α_{g} is gene-specific mutation rate, influenced by several additional known factors that can influence the underlying mutation rate for a given gene, including replication timing (R), expression level (X), open-chromatin state (C), and whether gene is an olfactory receptor (O). Effect of these factors is estimated from negative binomial regressions as described below.

In some embodiments, the synonymous mutation count y_{gs} of gene g and sample s with negative binomial regression, assuming common dispersion ϕ across genes, is modeled as:

$${y}_{g s} ∼ ZIP ( mean = {α}_{g} {b}_{s} {E}_{g ( s y n o n y m o u s )} , probability of extra zeros = {p}_{g} ) \\ where \\ ln ( {α}_{g} ) = {X}^{T} β , \\ logit ( {p}_{g} ) = {X}^{T} β , \qquad [ 5 ]$$

and where β and β′ are estimated by running a regression using all genes and all samples. X^{T} is a vector of relevant regressors including R, X, C, and O.

(3ii) Capture Unknown Factor Impact Through Maximum Likelihood Method

In equation [2] above, it is assumed that the mutation rate factors are only dependent on proposed regressors, but unknown mechanisms or biological factors can also impact mutation rate. Therefore, each gene is modeled as an independent zero-inflated Poisson process and the Maximum Likelihood estimation (MLE) (such as described above) is used to estimate gene-specific excessive zero probability p_{g} and [symbol] by maximizing equation [6] (below). For each gene, n is the number of samples in the training cohort, k_{g} is number of samples of n whose observed mutation count in gene g is 0, and Y_{g}={y_{g1}, y_{g2}, . . . , y_{gs}} are the synonymous mutation counts in different samples. In this step, the influencing factors (R, X, C, O) are not applicable.

$$\begin{matrix}P ( {Y}_{g} \vert λ , {p}_{g} ) = {{[ {p}_{g} + ( 1 - {p}_{g} ) {e}^{- λ} ]}^{{k}_{g}} [ ( 1 - {p}_{g} ) {e}^{- λ} ]}^{( n - {k}_{g} )} {\prod }_{j = {k}_{g} + 1}^{n} \frac{{λ}^{{y}_{i}}}{{y}_{i} !} \\ where & [ 6 ] \\ λ = {b}_{s} {E}_{g ( synonymous )} & [ 7 ]\end{matrix}$$

(3iii) Optimization of Gene-Specific Mutation Rate Factors

Because α_{g} is obtained by pooling all genes together, it is believed to capture the common trend of the influencing factors (R, X, C, O) on background mutation rate. On the contrary, it is believed that [symbol] is a gene-specific parameter from the observed data independent of the influencing factors. In some embodiments, [symbol] and α_{g} are not always the same, which could be caused by technical noise (e.g. errors in mutation calling algorithms) or reflect real biological mechanisms (e.g. factors influencing the background mutation rate that are not included in our regression model). In some embodiments, and due to the low number of somatic mutations in each gene, [symbol] is very vulnerable to technical noise. Thus, it is advantageous to find the optimized α_{g}′ by incorporating both parameters from negative binomial regression and those directly from gene-specific estimation. In some embodiments, the posterior probability of α_{g}′ is proportional to the likelihood times prior with σ estimated as equation [11]. The prior probability is chosen to constrain α_{g}′ to be centered at α_{g}. We maximize [8] to obtain the proper α_{g}′ for each gene.

$$\begin{matrix}P ( {α}_{g}^{′} \vert {Y}_{g} , \hat{α} , {α}_{g} ) ∝ P ( {α}_{g}^{′} \vert \hat{α} , {α}_{g} ) {\prod }_{s} P ( {y}_{g s} \vert {α}_{g}^{′} , {p}_{g} ) & [ 8 ] \\ {y}_{g s} ∼ ZIP ( {α}_{g} {b}_{s} {E}_{g} ( synonymous ) , {p}_{g} ) & [ 9 ] \\ P ( {α}_{g}^{′} \vert \hat{α} , {α}_{g} ) = \frac{1}{σ \sqrt{2 π}} {e}^{\frac{- {( ln {a}_{g}^{′} - ln {a}_{g} )}^{2}}{2 {σ}^{2}}} & [ 10 ]\end{matrix}$$

Where σ can be estimated by:

$$σ = \sqrt{\frac{1}{N} {Σ}_{g} - {( ln {α}_{g}^{′} - ln {α}_{g} )}^{2}} \qquad [ 11 ]$$

Then the ‘gene-specific estimation’ and ‘optimization of gene mean’ steps are repeated by replacing [symbol] with α_{g}′ to re-estimate dispersions until convergence is achieved. The estimated α_{g}′ and p_{g} are used in estimating the tumor mutational burden (step 350 of FIG. 3B).

In other embodiments, the steps described within PCT Publication No. WO/2017/181134 (the disclosure of which is hereby incorporated by reference herein in its entirety) may be used for deriving parameters for estimating tumor mutational burden.

Gaussian Mixture Model Training Module

In some embodiments, training data may be acquired using a Gaussian Mixture Model Training module 207. In some embodiments, the training module 207 uses acquired sequencing data, such as whole exome sequencing data or targeted panel sequencing data (including such data stored in storage system 240) to detect somatic mutations within the sequencing data, including SNV and INDEL. In some embodiments, the training module 207 employs the mutation identification module 203 to identify the somatic mutations in the acquired training data. In some embodiments, the training module 207 determines the tumor mutational burdens according to different methods, such as those described herein and using the tumor mutational burden estimation module 204. In other embodiments, the training module 207 utilizes those methods described within PCT Publication Nos. WO/2018/183928 and WO/2018/068028, the disclosures of which are hereby incorporated by reference herein in their entities. In some embodiments, the training data is stored within storage system 240. In some embodiments, the training data will be a cohort containing as least TMB for each sample in the cohort.

### Additional Embodiments

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Any of the modules described herein may include logic that is executed by the processor(s). “Logic,” as used herein, refers to any information having the form of instruction signals and/or data that may be applied to affect the operation of a processor. Software is an example of logic.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

The term “programmed processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user and a keyboard and a pointing device, e.g., a mouse or a trackball, by which the user can provide input to the computer. In some implementations, a touch screen can be used to display information and receive input from a user. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks). For example, the network can include one or more local area networks.

The computing system can include any number of clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Example of Identifying Cancer Subtypes within Sequencing Data

Overview

A tumor mutation burden method that utilizes an explicit background mutation model to predict TMB and to classify samples into biologically and clinically relevant subtypes defined by TMB is described below.

By analyzing publicly available TCGA data, it was discovered that log-transformed TMB can reveal three hidden cancer subtypes: TMB-Low, TMB-High, and the novel TMB-Extreme subtypes in colorectal, stomach and endometrial cancer (FIGS. 6A-6C). Each of these three cancer subtypes was observed to have distinguishable mutation profiles. A TMB-Low cancer subtype was observed in patients having a low mutation rate and patients whose sequencing data was depleted with mutations in the POLE and the dMMR pathway genes. A TMB-High cancer subtype included MSI-H patients and those patients characterized as having a high INDEL mutation rate. A TMB-Extreme cancer subtype was surprisingly discovered, where patients had an extremely high SNV mutation rate but low INDEL mutation rate, and where patients were enriched with non-synonymous mutations in the POLE gene (FIGS. 6A-6C). TMB-Extreme was previously obscured as it was classified as TMB-High, which hindered the discovery of a more accurate stratification for survival analysis.

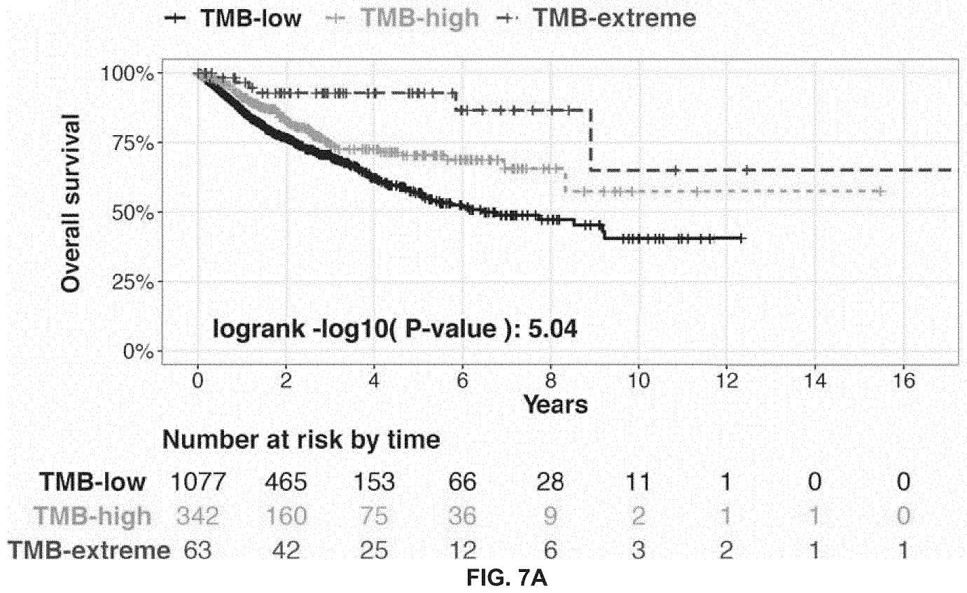

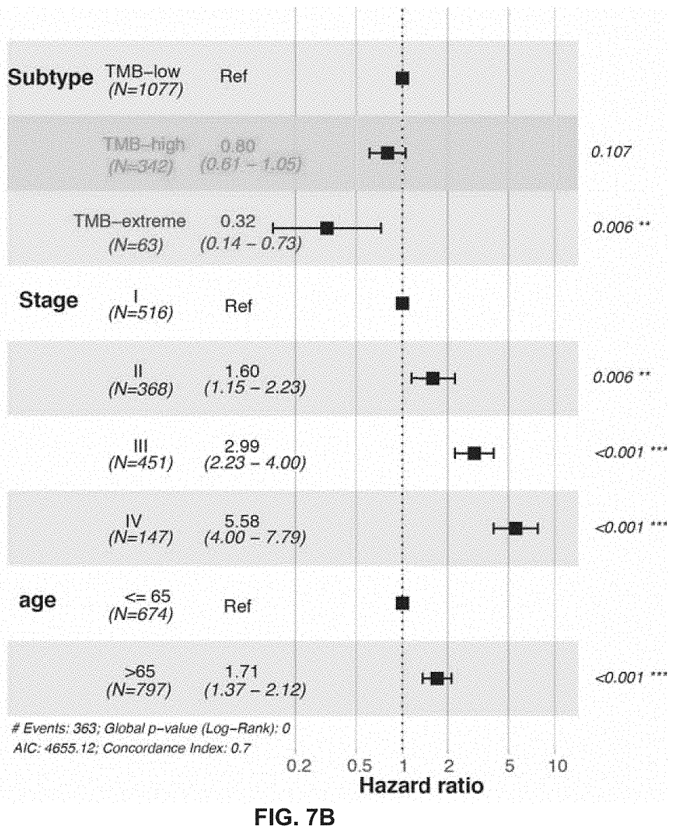

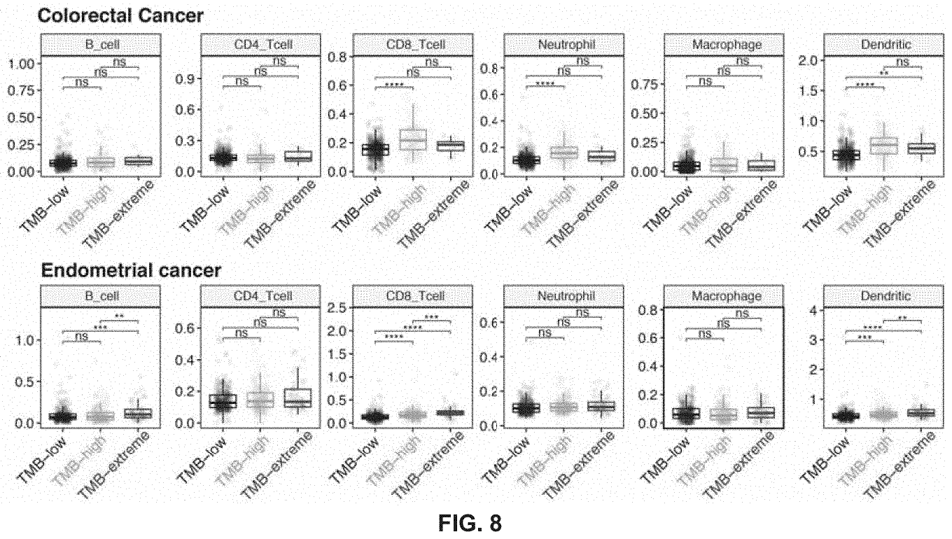

The survival outcome was investigated. It was observed that TMB-High and TMB-Extreme were associated with improved patient survival after considering age and stage (hazard ratio (HR) for TMB-High=0.8 with P-value=0.1; hazard ratio (HR) for TMB-Extreme=0.32 with P-value=0.006) (FIGS. 7A to 7B). TMB-Extreme showed a significantly lower hazard ratio than TMB-High, indicating better survival rate. Both TMB-High and TMB-Extreme were associated with higher infiltrating B cells, CD8 T cells and Dendritic cells in colorectal and endometrial cancer (FIG. 8).

### Introduction

Over the past 40 years, advances in next generation sequencing (NGS) technologies have provided an unprecedented opportunity to characterize cancer genomic landscapes and identify mutations relevant for diagnosis and therapy. It has been shown that cancers can be caused by an accumulation of genetic mutations in oncogenes or tumor suppressors, which lead to dysregulation of cell proliferation and survival (Vogelstein, B. et al. Cancer genome landscapes. Science 339, 1546-1558 (2013)). These mutations are known as “driver” mutations and they are believed to be under positive selection due to their contributions to tumorigenesis. However, only a very small fraction of the thousands of somatic mutations in a tumor sample are expected to be drivers. The remaining majority of somatic mutations are “passengers,” accumulated randomly with a background mutation rate during cancer progression (Iranzo, J., Martincorena, I. & Koonin, E. V. Cancer-mutation network and the number and specificity of driver mutations. Proc. Natl. Acad. Sci. U.S.A. 115, E6010-E6019 (2018)).

Moreover, analyses of a large collection of cancer genomes have shown that the background mutation rate varies, by as much as about 1000 fold, among different cancer types, within patients having a single cancer type, and within genomic regions (Lawrence, M. S. et al. Mutational heterogeneity in cancer and the search for new cancer-associated genes. Nature 499, 214-218 (2013)). Association analyses between mutation rates and genomic features have been used to identify regional mutation heterogeneity in cancers (Chapman, M. A. et al. Initial genome sequencing and analysis of multiple myeloma. Nature 471, 467-472 (2011); Hodgkinson, A. & Eyre-Walker, A. Variation in the mutation rate across mammalian genomes. Nature Publishing Group 12, 756-766 (2011); Pleasance, E. D. et al. A comprehensive catalogue of somatic mutations from a human cancer genome. Nature 463, 191-196 (2010)). For example, gene expression level has been found to be negatively correlated with somatic mutation rate (Iranzo, J., Martincorena, I. & Koonin, E. V. Cancer-mutation network and the number and specificity of driver mutations. Proc. Natl. Acad. Sci. U.S.A. 115, E6010-E6019 (2018)). It is believed that late replicating regions have higher mutation rates.

Similar correlations have been identified for germline mutation rates (Stamatoyannopoulos, J. A. et al. Human mutation rate associated with DNA replication timing. Nat. Genet. 41, 393-395 (2009); Koren, A. et al. AR TICLE Differential Relationship of DNA Replication Timing to Different Forms of Human Mutation and Variation. The American Journal of Human Genetics 91, 1033-1040 (2012)). It is also believed that the mutation rate for each tri-nucleotide context is different, as a result of diverse mutation signatures on cancer genome through different mutagenic processes (Australian Pancreatic Cancer Genome Initiative et al. Signatures of mutational processes in human cancer. Nature 500, 415-421 (2013)).

Cancer mutational rates can also vary widely even across patients within the same cancer type, such as ranging from 0.01 per megabase (Mb) to 300 per Mb in stomach cancer and from less than 1 per Mb to more than 700 per Mb in endometrial cancer (Australian Pancreatic Cancer Genome Initiative et al. Signatures of mutational processes in human cancer. Nature 500, 415-421 (2013)). A patient with a high somatic mutation rate is referred to as having the hypermutated phenotype. It is believed that the possible root causes for increased background mutation rate includes increased DNA synthesis or repair errors and increased DNA damage (Roberts, S. A. & Gordenin, D. A. Hypermutation in human cancer genomes: footprints and mechanisms. Nat. Rev. Cancer 14, 786-800 (2014)). About 100,000 polymerase errors occur during DNA replication every time a cell divides, thus correction mechanisms for DNA replication are essential for genome stability (Nebot-Bral, L. et al. Hypermutated tumours in the era of immunotherapy: The paradigm of personalised medicine. Eur. J. Cancer 84, 290-303 (2017)). This is accomplished by a collaborative effort of the 3′-5′ exonuclease activity of polymerase epsilon (POLE) and delta (POLD1), the MMR system and other DNA repair genes such as BRCA (Rayner, E. et al. A panoply of errors: polymerase proofreading domain mutations in cancer. Nat. Rev. Cancer 16, 71-81 (2016); Jiricny, J. The multifaceted mismatch-repair system. Nat. Rev. Mol. Cell Biol. 7, 335-346 (2006); Zámborszky, J. et al. Loss of BRCA1 or BRCA2 markedly increases the rate of base substitution mutagenesis and has distinct effects on genomic deletions. Oncogene 36, 746-755 (2017)).

It is believed that deleterious mutations in POLE, POLD1, and MMR system defects may lead to hypermutated phenotype (Lawrence, M. S. et al. Mutational heterogeneity in cancer and the search for new cancer-associated genes. Nature 499, 214-218 (2013); Roberts, S. A. & Gordenin, D. A. Hypermutation in human cancer genomes: footprints and mechanisms. Nat. Rev. Cancer 14, 786-800 (2014); Nebot-Bral, L. et al. Hypermutated tumours in the era of immunotherapy: The paradigm of personalised medicine. Eur. J. Cancer 84, 290-303 (2017); Campbell, B. B. et al. Comprehensive Analysis of Hypermutation in Human Cancer. Cell 171, 1042-1056.e10 (2017); Finocchiaro, G., Langella, T., Corbetta, C. & Pellegatta, S. Hypermutations in gliomas: a potential immunotherapy target. Discov Med 23, 113-120 (2017)). Seven genes have been identified as essential components for MMR system, including MLH1, MLH3, MSH2, MSH3, MSH6, PMS1, PMS216,20. Besides DNA synthesis/repair errors, increased DNA lesion will also lead to hypermutation phenomenon. For example, UV radiation can increase rate of C->T at a dipyrimidine sites, which is a risk factor for skin cancer4. Ingredients of tobacco can cause increased G->T transversions among smokers in lung cancer and bladder cancer (Govindan, R. et al. Genomic landscape of non-small cell lung cancer in smokers and never-smokers. Cell 150, 1121-1134 (2012)). It is believed that oxidative DNA damage, which is caused by products from cell metabolism or environmental intakes, is likely to be one of main causes of age-dependent mutations and cancers (Longo, V. D., Lieber, M. R. & Vijg, J. Turning anti-ageing genes against cancer. Nat. Rev. Mol. Cell Biol. 9, 903-910 (2008)).

As noted herein, immunotherapy targeting immune checkpoint inhibitors, such as programmed cell death protein 1 (PD-1) with its receptor (PD-L1) and cytotoxic T lymphocyte-associated antigen 4 (CTLA-4), showed remarkable clinical benefits for various advanced cancers (Wolchok, J. D. et al. Overall Survival with Combined Nivolumab and Ipilimumab in Advanced Melanoma. N. Engl. J. Med. 377, 1345-1356 (2017); Borghaei, H. et al. Nivolumab versus Docetaxel in Advanced Nonsquamous Non-Small-Cell Lung Cancer. N. Engl. J. Med. 373, 1627-1639 (2015); Aggen, D. H. & Drake, C. G. Biomarkers for immunotherapy in bladder cancer: a moving target. 1-13 (2017). doi:10.1186/s40425-017-0299-1; Saleh, K., Eid, R., Haddad, F. G., Khalife-Saleh, N. & Kourie, H. R. New developments in the management of head and neck cancer &ndash; impact of pembrolizumab. TCRM Volume 14, 295-303 (2018)). While it is believed that these immune checkpoint blockade cancer therapies have dramatically improved the efficacy of immunotherapy, only a fraction of patients are responsive to the treatment. Therefore, to maximize the therapeutic benefits, and as noted herein, it is critical to identify predictive biomarkers to distinguish the responsive and nonresponsive patients.

PD-L1 expression level and microsatellite instability-high (MSI-H) have been developed to be predictive biomarkers for the clinical outcome of anti-PD-L1 therapy (Reck, M. et al. Pembrolizumab versus Chemotherapy for PD-L1-Positive Non-Small-Cell Lung Cancer. N. Engl. J. Med. 375, 1823-1833 (2016); Le, D. T. et al. PD-1 Blockade in Tumors with Mismatch-Repair Deficiency. N. Engl. J. Med. 372, 2509-2520 (2015)). Microsatellite instability (MSI) is a phenotype of an accumulation of deletions/insertions in repetitive DNA tracts, called microsatellites, in cancer. Similar to hypermutation, evidences have indicated that MSI is a mutator phenotype resulted from a deficient MMR system (Laghi, L., Bianchi, P. & Malesci, A. Differences and evolution of the methods for the assessment of microsatellite instability. Oncogene 27, 6313-6321 (2008); Vilar, E. & Gruber, S. B. Microsatellite instability in colorectal cancer—the stable evidence. Nat Rev Clin Oncol 7, 153-162 (2010)).

Hypermutation was first associated with response to CTLA-4 blockade therapy in 2014 and PD-1 blockade therapy in 2015 (Snyder, A., Wolchok, J. D. & Chan, T. A. Genetic basis for clinical response to CTLA-4 blockade. N. Engl. J. Med. 372, 783-783 (2015); Rizvi, N. A. et al. Mutational landscape determines sensitivity to PD-1 blockade in non-small cell lung cancer. Science 348, 124-128 (2015)). The underlying hypothesis is that greater quantities of neoantigens from a hypermutated tumor leads to stronger adaptive immune response (Nebot-Bral, L. et al. Hypermutated tumours in the era of immunotherapy: The paradigm of personalised medicine. Eur. J. Cancer 84, 290-303 (2017)).

Tumor mutational burden, which is a measure of the abundance of somatic mutations, has since become a new, promising biomarker for both prognosis and immunotherapy (Samstein, R. M. et al. Tumor mutational load predicts survival after immunotherapy across multiple cancer types. Nat. Genet. 51, 202-206 (2019); Hellmann, M. D. et al. Nivolumab plus Ipilimumab in Lung Cancer with a High Tumor Mutational Burden. N. Engl. J. Med. 378, 2093-2104 (2018); Van Allen, E. M. et al. Genomic correlates of response to CTLA-4 blockade in metastatic melanoma. Science 350, 207-211 (2015); Hugo, W. et al. Genomic and Transcriptomic Features of Response to Anti-PD-1 Therapy in Metastatic Melanoma. Cell 165, 35-44 (2016)). Nevertheless, multiple challenges still hinder the adoption of TMB for clinical decision making. The current well-accepted TMB measurement requires counting the non-synonymous somatic mutations in a paired tumor-normal sample using whole-exome sequencing (WES). However, clinical diagnostics based on sequencing technologies still rely heavily on targeted panel sequencing. Although studies have shown panel-based TMB measurement were highly correlated with WES-based TMB, the inconsistency between these two measurements have been observed (Samstein, R. M. et al. Tumor mutational load predicts survival after immunotherapy across multiple cancer types. Nat. Genet. 51, 202-206 (2019); Chalmers, Z. R. et al. Analysis of 100,000 human cancer genomes reveals the landscape of tumor mutational burden. 1-14 (2017). doi:10.1186/s13073-017-0424-2; de Velasco, G. et al. Targeted genomic landscape of metastases compared to primary tumours in clear cell metastatic renal cell carcinoma. Br. J. Cancer 118, 1238-1242 (2018); Garofalo, A. et al. The impact of tumor profiling approaches and genomic data strategies for cancer precision medicine. Genome Med 8, 1023 (2016)).

One reason for this inconsistency is believed to be that targeted panel sequencing might overestimate TMB due to its enrichment of driver mutations and mutation hot spots. In facts, WES-based TMB is believed to be more indicative of the overall background mutation rate because of few incidences of driver mutations and hot spots in whole exome. In order to avoid overestimating TMB, various filtering strategies have been applied. For example, Foundation Medicine used COSMIC to filter out driver mutations and add synonymous mutations to reach an agreement with WES-based TMB (Chalmers, Z. R. et al. Analysis of 100,000 human cancer genomes reveals the landscape of tumor mutational burden. 1-14 (2017)). These arbitrary filters are dependent on frequently updated databases, worsening the inconsistency, reproducibility and robustness of the calculation. Another non-negligible challenge is the relatively arbitrary selection of the TMB-high cutoff such as 10 or 20 per Mb or top 10% or 20% quantile (Isharwal, S. et al. Prognostic Value of TERT Alterations, Mutational and Copy Number Alterations Burden in Urothelial Carcinoma. Eur Urol Focus (2017); Burden. N. Engl. J. Med. 378, 2093-2104 (2018); Chalmers, Z. R. et al. Analysis of 100,000 human cancer genomes reveals the landscape of tumor mutational burden. 1-14 (2017)). Although these thresholds were enough to illustrate the predictive value of TMB as a biomarker, an appropriate TMB cutoff derived from sophisticated studies or clinical trials is needed, as noted herein.

In order to improve the robustness of TMB measurement and TMB subtype classification, we proposed a novel method called ecTMB (estimation and classification of TMB) (see, e.g., FIGS. 5A-5C). Because WES-based TMB is akin to the overall background mutation rate, we built a statistical model using a Bayesian framework for TMB prediction. As described in detail herein, the model takes into account the heterogeneous mutation contexts in cancer and other influencing factors to estimate the sample- and gene-specific background mutation rates, which can systematically reduce driver-mutation effects and include synonymous mutations in the estimation. Again, as noted herein, by analyzing publicly available TCGA data, it was discovered that log-transformed TMB could reveal three hidden cancer subtypes: TMB-Low, TMB-High, and the novel TMB-Extreme subtypes in colorectal, stomach and endometrial cancer (FIGS. 6A-6C).

Based on this observation, ecTMB with a Gaussian Mixture Model was extended to classify samples by the aforementioned cancer subtypes. Our method was evaluated using WES data from The Cancer Genome Atlas (TCGA). The cancer types included in our analyses were colon adenocarcinoma (COAD), rectal adenocarcinoma (READ), stomach adenocarcinoma (STAD), and uterine corpus endometrioid carcinoma (UCEC). Based on previous analysis, READ and COAD are often combined for analysis due to their similarity (Network, T. C. G. A. Comprehensive molecular characterization of human colon and rectal cancer. Nature 487, 330-337 (2012)). Additionally, the availability of MSI status of these cancer types provided us an opportunity to investigate the association between TMB and MSI status.

Dataset

By way of example, somatic mutations generated by MuTect2 (in reference version of hg38) and clinical profiles of TCGA samples may be downloaded from a publicly available database (see, e.g. Grossman, R. L. et al. Toward a Shared Vision for Cancer Genomic Data. N. Engl. J. Med. 375, 1109-1112 (2016)). In some embodiments, formalin-fixed paraffin-embedded (FFPE) tissue samples are excluded from downstream analysis. Tumor-infiltrating immune cell abundance may also be downloaded (see Li, T. et al. TIMER: A Web Server for Comprehensive Analysis of Tumor-Infiltrating Immune Cells. Cancer Research 77, e108-e110 (2017)). Replication timing, expression level, and open-chromatin state of all protein-coding genes may be extracted (see Lawrence, M. S. et al. Mutational heterogeneity in cancer and the search for new cancer-associated genes. Nature 499, 214-218 (2013)).

Whole Exome Annotation

In some embodiments, Ensembl 81 GRC38 may be downloaded and processed to generate all possible mutations and their functional impacts for the genome. First, every genomic base in coding regions was changed to the other three possible nucleotides and the Variant Effect Predictor (VEP) was used to annotate their functional impacts. Each variant's functional impact was picked following the criteria: biotype>consequence>transcript length. Each variant's tri-nucleotide contexts, including before and after mutated base, and corresponding amino acid positions relative to protein length were reported.

Tumor Mutational Burden Estimation and Subtype Classification

Based on the obtained sequencing data, a tumor mutational burden was estimated using the processes described herein. A log-transformed of the estimated tumor mutational burden was then modeled using a Gaussian mixture model such as described herein. Modeling provided the results identified below.

Background Mutation Prediction by BMR Model

Within each cancer type, WES data from two-thirds of the samples was used for training to determine parameters for the background mutation model. Background mutations were predicted using the equations below for non-synonymous mutation and for synonymous mutation in both the training set and the remainder of the testing set.

$$\# of expected background non \text{-} synonymous mutations = {α}_{g} {b}_{s} {E}_{g ( non \text{-} synoymous )}$$

$$\# of expected background synonymous mutations = {α}_{g} {b}_{s} {E}_{g ( s y n o y m o u s )}$$

Cancer Subtype Classification and Characterization

Within each cancer type (colorectal, endometrial and stomach cancer), log-transformed TMBs, either defined by the total number of mutations per Mb or the number of non-synonymous mutation per Mb, were modeled using a Gaussian Mixture Model as described herein. Each sample was assigned to one of TMB-low, TMB-high and TMB-Extreme classes based on its assignment score. For each sample, indel incidence, estimated immune cell abundance and non-synonymous mutation existence (occurrence >1) in POLE and dMMR pathway genes including MLH1, MLH3, MSH2, MSH3, MSH6, PMS1, and PMS2 were summarized. Mutations of POLE and MMR system genes were plotted using maftools (Mayakonda, A., Lin, D.-C., Assenov, Y., Plass, C. & Koeffler, H. P. Maftools: efficient and comprehensive analysis of somatic variants in cancer. Genome Res. 28, 1747-1756 (2018)).

Cancer Survival Analysis

Kaplan-Meier survival analysis was used to estimate the association of cancer subtype with the overall survival of patients with colorectal, endometrial and stomach cancers aggregated data. Furthermore, we performed proportional hazard ratio analysis using the coxph function in R, including age, stage and subtypes as covariates. The significances of the covariates were assessed by Wald tests. Overall survival was calculated from the date of initial diagnosis of cancer to disease-specific death (patients whose vital status is termed dead) and months to last follow-up (for patients who are alive).

Tmb Prediction for Panels

In order to evaluate ecTMB prediction for panels, in silico analyses were performed. A panel coordinates bed file of Illumina TruSight Tumor 170 was downloaded (panel size 524 kb) from Illumina's website (https://support.illumina.com/content/dam/illumina-support/documents/downloads/productfiles/trusight/trusight-tumor-170/tst170-dna-targets.zip). The gene lists of FoundationOne CDx and Integrated Mutation Profiling of Actionable Cancer Targets (MSK-IMPACT) were download from Foundation Medicine website (https://www.foundationmedicine.com/genomic-testing/foundation-one-cdx) and an FDA document (https://www.accessdata.fda.gov/cdrh_docs/reviews/den170058.pdf), respectively. Corresponding panel coordinate beds were generated based on gene lists for FoundationOne CDx and MSK-IMPACT. The final sizes of FoundationOne CDx and MSK-IMPACT panels were 5.4 Mb and 10 Mb, respectively, which may be larger than the exact commercial panels. Mutations located in a given panel were selected to represent the mutations which can be detected by this targeted panel sequencing. Within each cancer type, WES data from two-thirds of samples were used for training to determine background mutation model parameters. In-silico targeted panel sequencing data from one-third of samples were used for testing. Both ecTMB and counting methods were applied to test data. A Bland-Altman analysis was performed using R package blandr.

Cluster Cancer Types Based on Tmb Distribution

WES mutation data for 29 cancer types was downloaded from GDC. For each cancer type, the density of log-transformed TMB was generated by bin=1. Then we used K-means clustering method to group cancer types to 5 clusters based on the similarity of the log-transformed TMB density. In each cluster, the mutation data was aggregated for further analysis.

Results

Background Mutation Modeling

Background mutation rate (BMR) modeling is one of major challenges for driver mutation detection. Multiple methods have been developed to model BMR. MutSigCV applies genomic features to estimate BMR44 and DrGaP builds a Bayesian framework to take 11 mutation types into consideration for BMR estimation (Hua, X. et al. DrGaP: a powerful tool for identifying driver genes and pathways in cancer sequencing studies. Am. J. Hum. Genet. 93, 439-451 (2013)). However, cancer mutational heterogeneity is far more complex, including differences among samples, genomic regions and tri-nucleotide contexts. Therefore, we developed a novel method to explicitly model the BMR in a sample- and gene-specific manner, taking into account both known and unknown influencing factors.

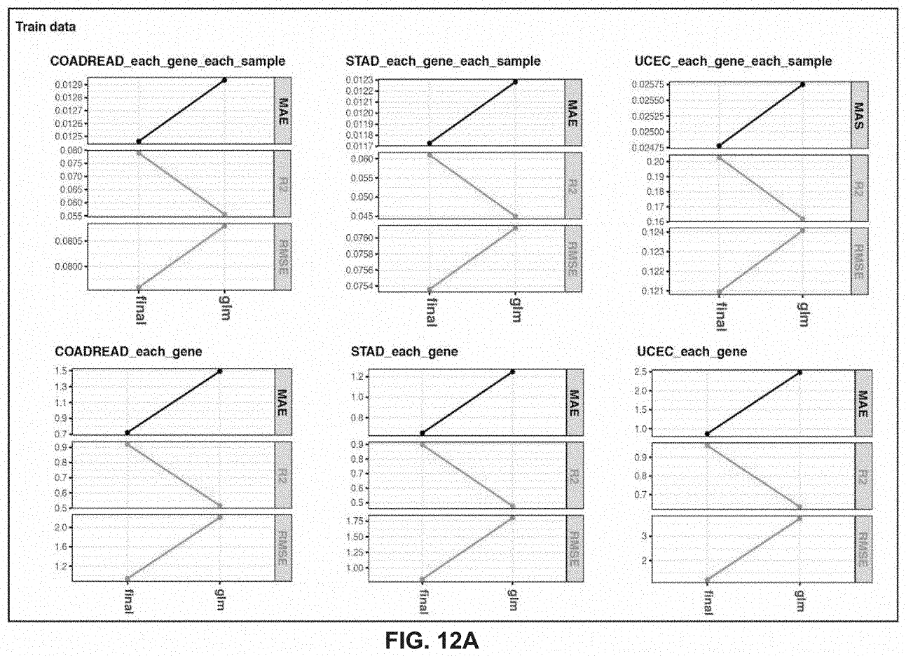

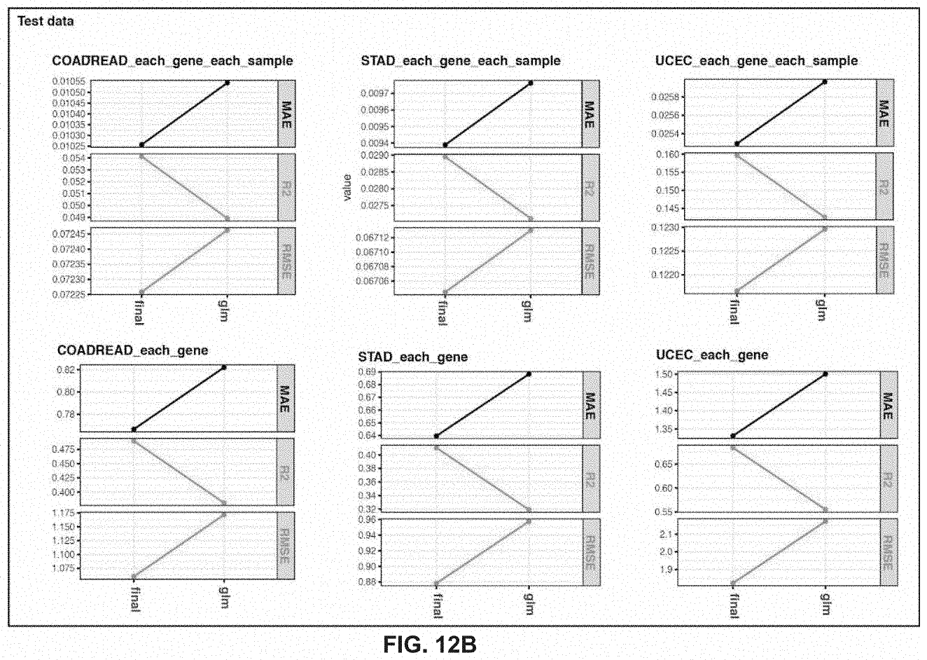

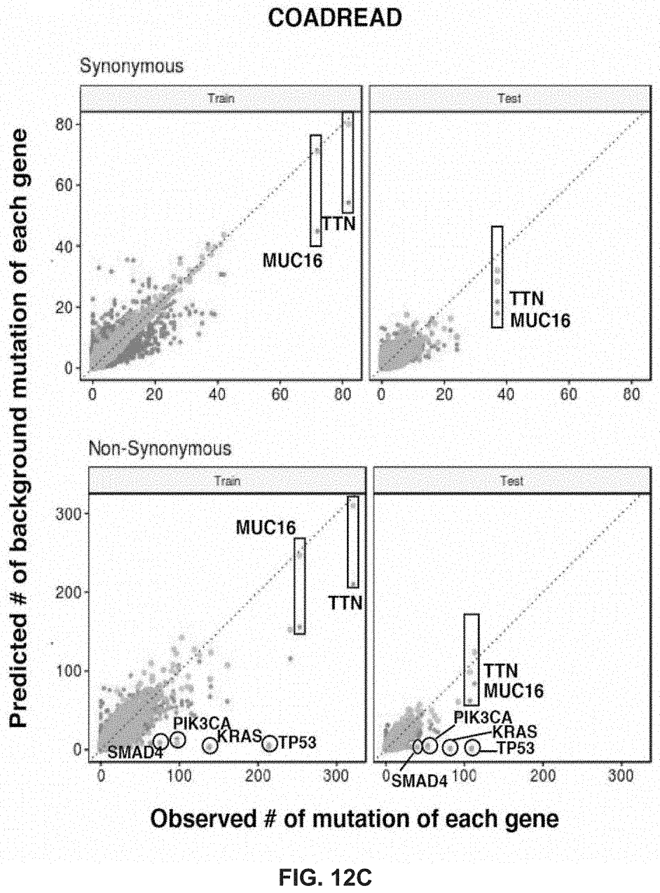

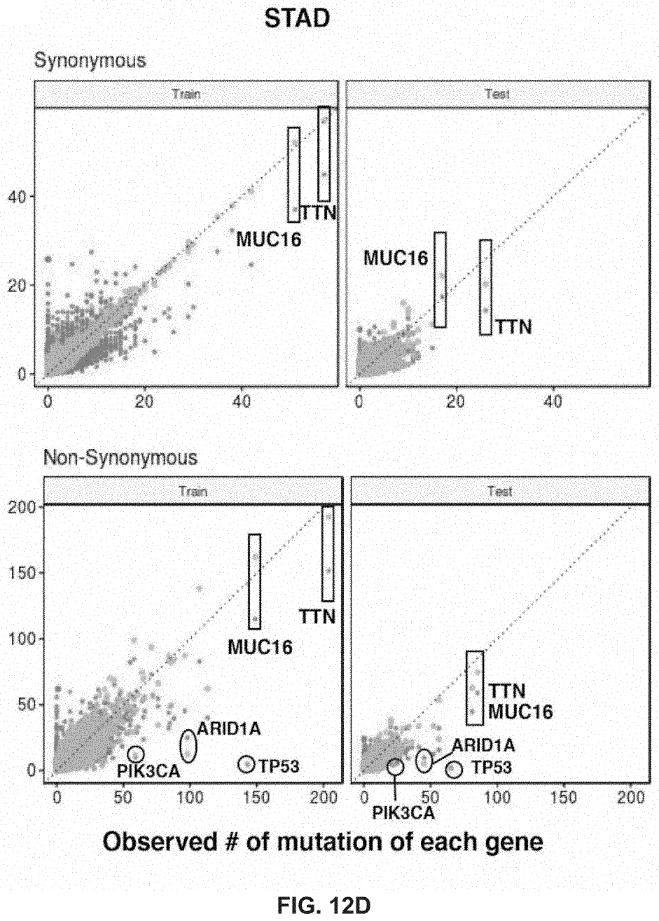

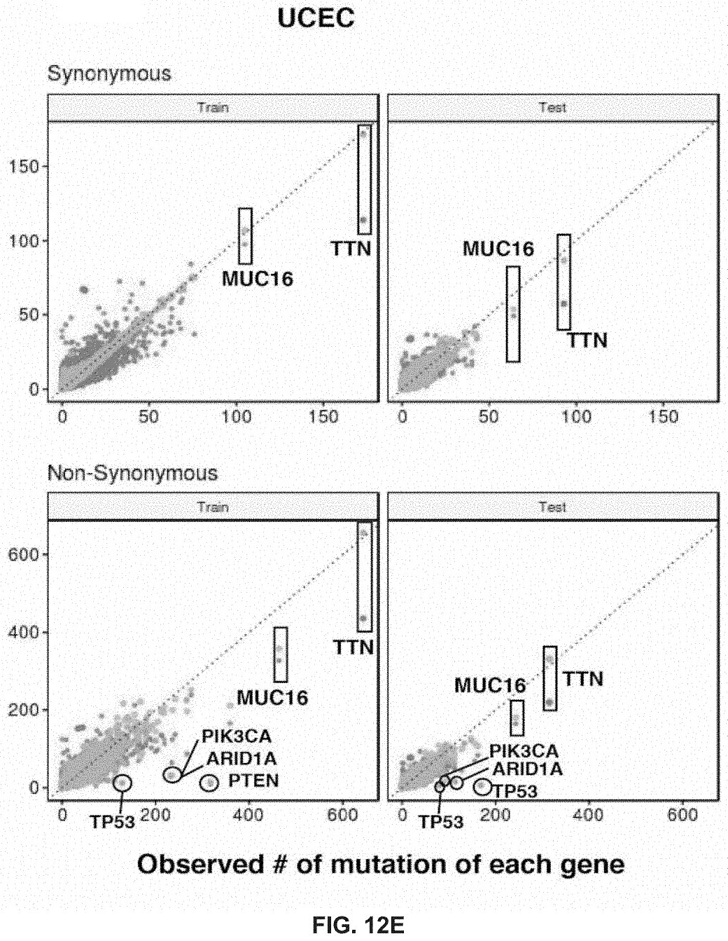

The occurrence of silent mutations was assumed to follow the BMR without selection pressures; whereas the number of background somatic mutations follows a negative binomial distribution. In order to incorporate all known factors, e.g. tri-nucleotide context, gene composition, sample mutational burden, gene expression level, and replication timing, a Generalized Linear Model (GLM) was used to estimate the general impact of these factors by pooling genes together (FIG. 5B). In order to evaluate our model, we divided samples corresponding to each cancer type into training and testing sets with a 70%:30% split. Training sets ware used to estimate the model parameters, which then could be used to predict the number of mutations for each gene of each sample based on the negative binomial, as described herein. Because of the assumption that synonymous mutations were accumulated with a BMR, the comparison of a predicted number of synonymous mutations against an observed number of synonymous mutations can be used to measure the performance of model. We found that the GLM model could not explain all of the variations in the observed number of synonymous mutations. For example, membrane-associated mucin (MUC16) and titin (TTN), which are two suspicious false-positive driver genes (Lawrence, M. S. et al. Mutational heterogeneity in cancer and the search for new cancer-associated genes. Nature 499, 214-218 (2013)), had a much lower predicted number of synonymous mutations than the real observations in both training and testing sets (FIG. 12). It was therefore assumed that there might be unknown sequencing or biological factors influencing BMR.

To handle unknown factors, each gene was modeled as an independent negative binomial process as the second step. The final adjusted gene-specific background mutation rates were then generated through a Bayesian framework to consolidate the estimators from the two previous steps (such as according to the methods described herein) (see also FIG. 5B). Compared with the prediction of synonymous mutations from GLM, the final model improved the R-squared value from 0.5 to about 0.9 in the training set and from 0.3 to about 0.6 in the testing set, and further reduced the mean absolute error (MAE) and the root mean square error (RMSE). Meanwhile, synonymous/non-synonymous mutation predictions for MUC16 and TTN became much closer to observed values (FIG. 12). These results displayed an improved performance when the approach described herein was applied.

A driver gene was expected to possess a higher non-synonymous mutation frequency relative to its BMR due to the positive selection. Indeed, a couple of well-known cancer-specific driver genes whose observed number of non-synonymous mutations were much higher than predicted background ones were discovered. Examples of those driver genes included TP53, KRAS, PIK3CA and SMAD4 in colorectal cancer (Network, T. C. G. A. Comprehensive molecular characterization of human colon and rectal cancer. Nature 487, 330-337 (2012)), TP53, ARID1A and PIK3CA in stomach cancer (Cui, J. et al. Comprehensive characterization of the genomic alterations in human gastric cancer. Int. J. Cancer 137, 86-95 (2015)), and PTEN, ARID1A, PIK3CA and TP53 in endometrial cancer (Cancer Genome Atlas Research Network et al. Integrated genomic characterization of endometrial carcinoma. Nature 497, 67-73 (2013)) (see FIG. 12). In summary, these results demonstrated that the disclosed method could accurately model background mutations and therefore systematically reduce driver gene impacts.

TMB Prediction

There were three determinants for BMR within the model described herein, namely sequence composition, gene-specific BMR and sample-specific BMR. From the training process described above, gene-specific BMRs could be estimated under the assumption that the sample-specific BMR of a sample could be calculated as either the number of all mutations per Mb or the number of non-synonymous mutations per Mb. Therefore, sample-specific BMR was equivalent to TMB. Here, we used the number of non-synonymous mutation as the TMB for the following TMB prediction and classification. With determined gene-specific BMRs from training set as described above, sample-specific BMR for a new sample could be estimated using Maximum Likelihood Estimation (MLE) through modeling each gene as an independent Negative Binomial process (see also FIG. 5B).

Using testing sets, we firstly evaluated how good TMB predictions by ecTMB were when all mutations, i.e. non-synonymous mutations as well as synonymous mutations, from WES were used. The standard TMB measurement, which ecTMB was compared against, was WES-based TMB calculated by the number of non-synonymous mutations divided by a sequenced genomic region size. The TMB varied largely, ranging from about 0.01 per Mb to about 760 per Mb in training and testing sets. The majority of samples (76%) had a TMB less than about 10 per Mb. Therefore, in order to deal with the large dynamic range of data and to avoid the mean absolute difference being determined only by large numbers, we presented performance measures with log-transformed values along with non-log-transformed ones. Correlation coefficient (R) is widely used to assess the agreement of TMB measurements among assays. However, a high correlation does not mean any two methods agree since R measures the strength of a relation between two variables, but not the exact agreement between them (Doğan, N. Ö., Bland-Altman analysis: A paradigm to understand correlation and agreement. Turk J Emerg Med 18, 139-141 (2018)). In order to comprehensively assess the agreement between ecTMB prediction and WES-based standard TMB calculation, we not only used the correlation coefficient, but also measured MAE and RMSE; and performed Bland-Altman analyses. It is believed that the Bland-Altman analysis is a widely used method to assess the agreement between two different assays, providing a bias measurement (mean difference), the limits of agreement and 95% confidence intervals for these measurements (Doğan, N. Ö.). It was found that the TMB predictions by ecTMB were highly concordant with standard TMB calculations in both correlation (correlation coefficient >0.998) and absolute error (MAE<1.833 in linear scale and MAE<0.063 in log scale) levels.

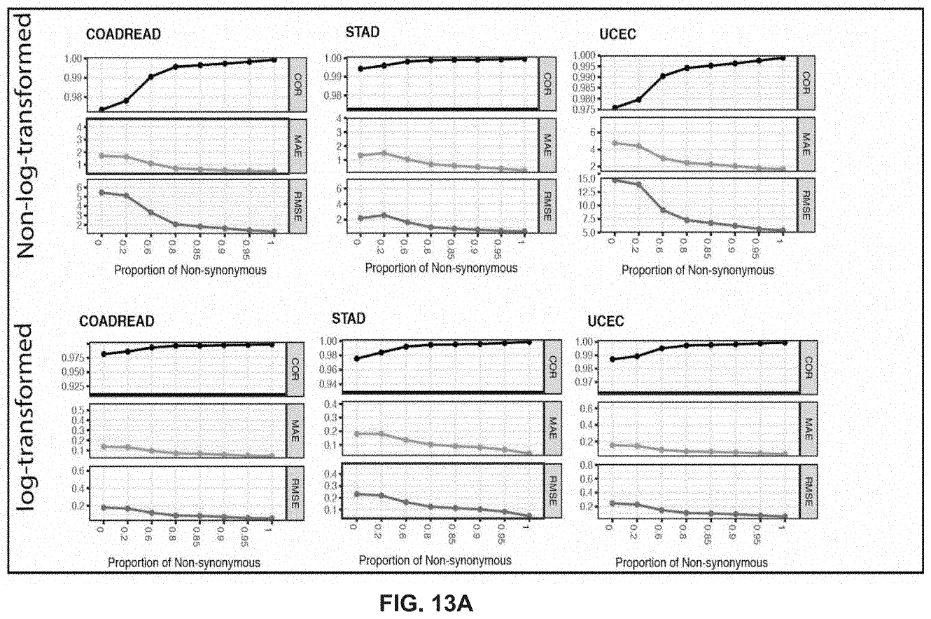

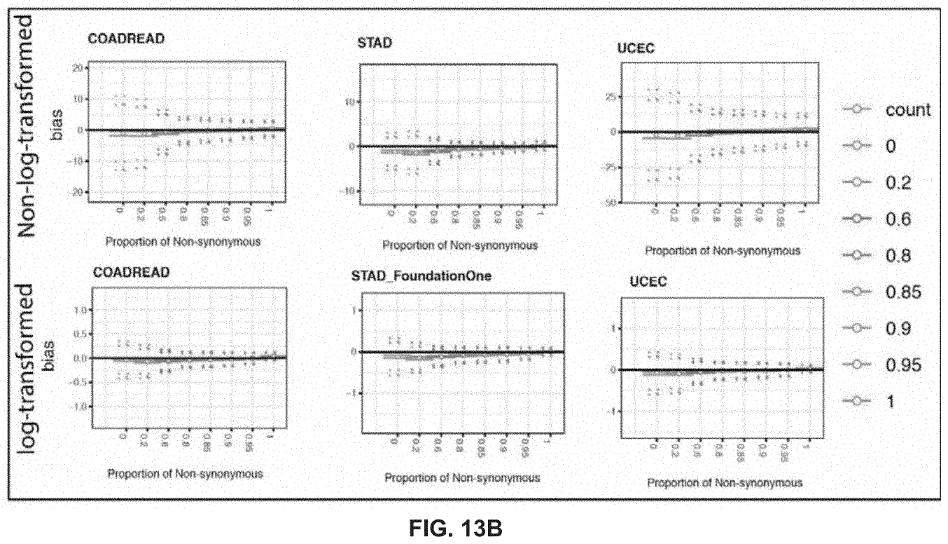

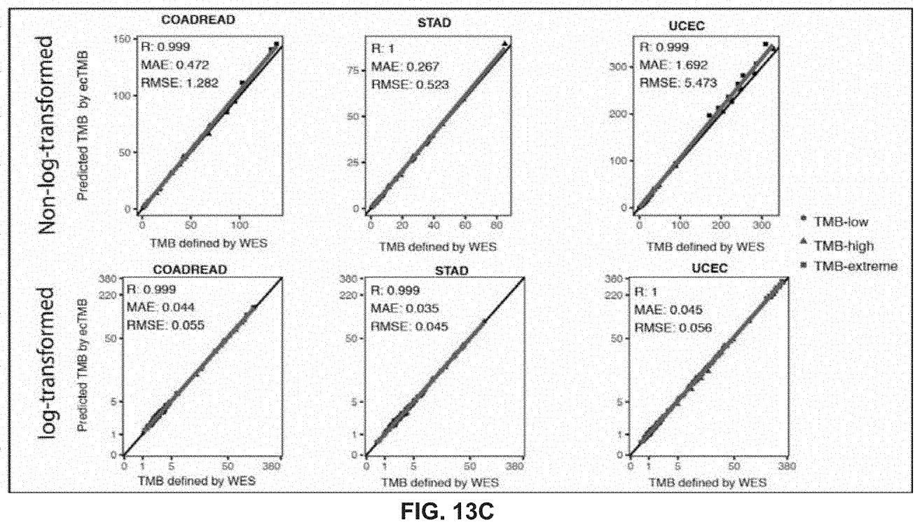

ecTMB can use synonymous mutations for TMB prediction since synonymous mutations follow the background mutation accumulation. Meanwhile, it is also able to incorporate non-synonymous mutations, most of which follow the BMR as well. The impact of including non-synonymous mutations from different proportions of genes was further assessed. Genes were ranked based on mutation frequency in training sets in each cancer types and non-synonymous mutations from least mutated genes (bottom 0%, 20%, 60%, 80%, 85%, 90%, 95% and 100%) were added to the prediction. In all, comparison across different proportions of non-synonymous mutations indicated that predictions with only synonymous mutations already had a great concordance with WES-based standard TMB with R>0.975 and almost 0 bias. However, the addition of non-synonymous mutations further improved the concordance, with R>0.999 and 0 bias when all non-synonymous mutations were used (see FIGS. 13A and 13B). With reference to FIG. 13B, for a set of n samples, two assays are performed on each sample, resulting in 2n data points. Each of the n samples is then represented on the graph by assigning the mean of the two measurements as the x-value, and the difference between the two values as the y-value. Fixed bias (d): the mean value of the difference differs significantly from 0 on the basis of a 1-sample t-test: Standard error of bias estimation (mean difference): √(var(y)/n); Upper and lower limit of 95% differences: d±(1.96*sd(y)); Standard error for upper and lower limit of 95% difference: √(3*var(y)/n).

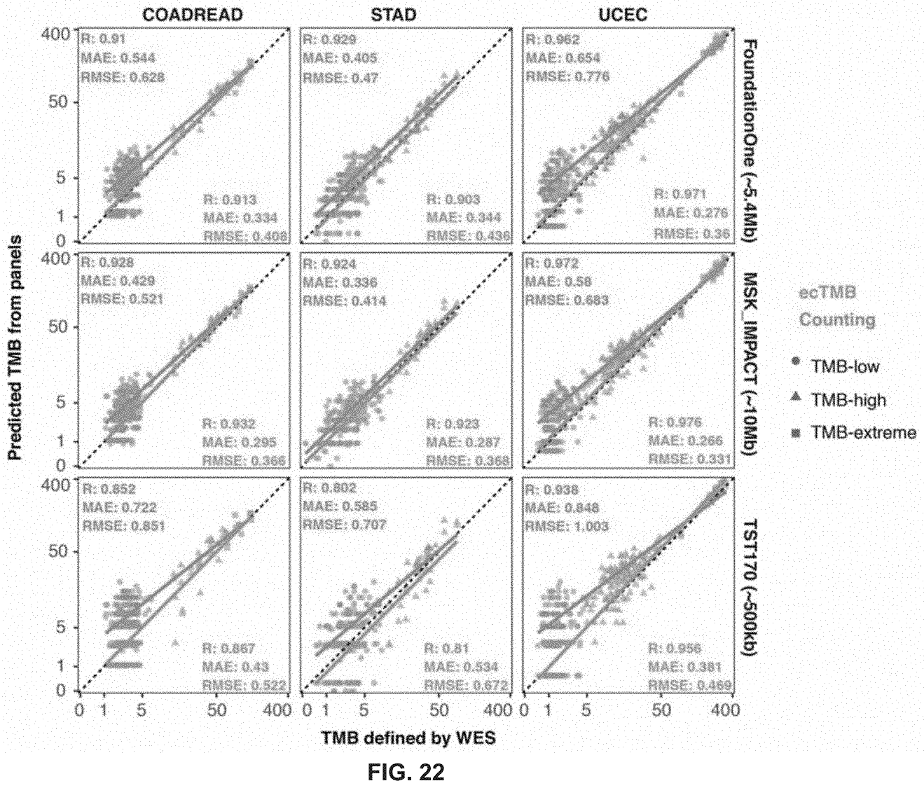

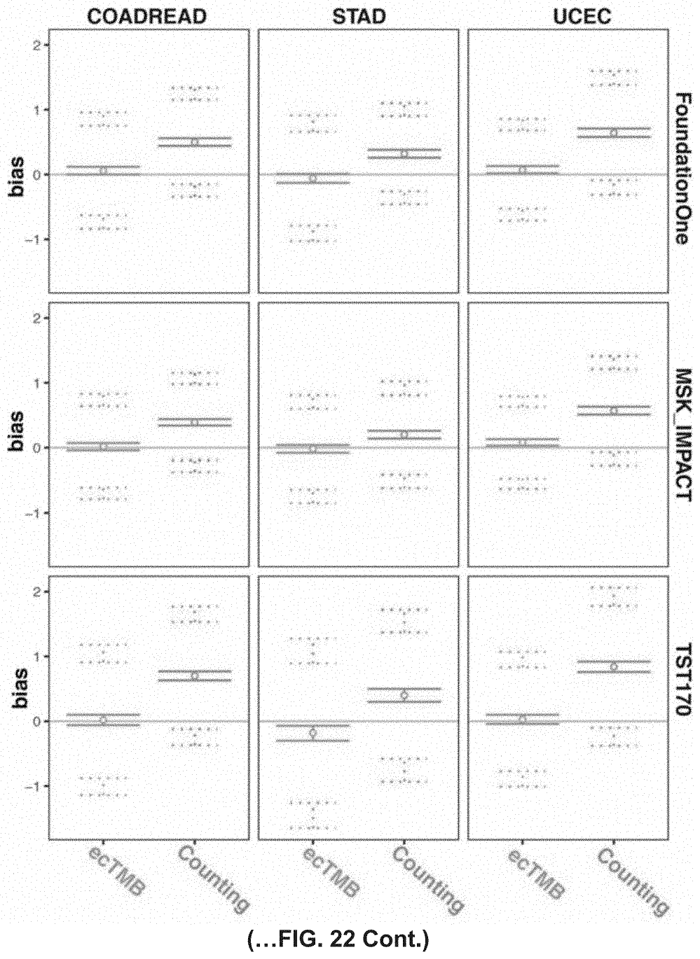

An in-silico assessment of panel-based TMB prediction was further conducted by the counting method and ecTMB on three cancer panels, including FoundationOne CDx, Integrated Mutation Profiling of Actionable Cancer Targets (MSK-IMPACT) 50 and Illumina TruSight Tumor 170 (TST170). Due to the lack of exact panel coordinates of FoundationOne CDx and MSK-IMPACT, the sizes of the panels converted from gene lists were larger than the real commercial panels. Only the mutations covered by each panel were used for the panel-based TMB prediction. A high correlation between WES-based standard TMB and panel-based TMB through simply counting the number of non-synonymous mutations was detected. But, Bland-Altman analysis showed the significant biases (>0) of panel-based TMB by counting, indicating an over-estimation especially for low TMB samples (FIG. 22 and FIGS. 6A, 6B, and 6C).

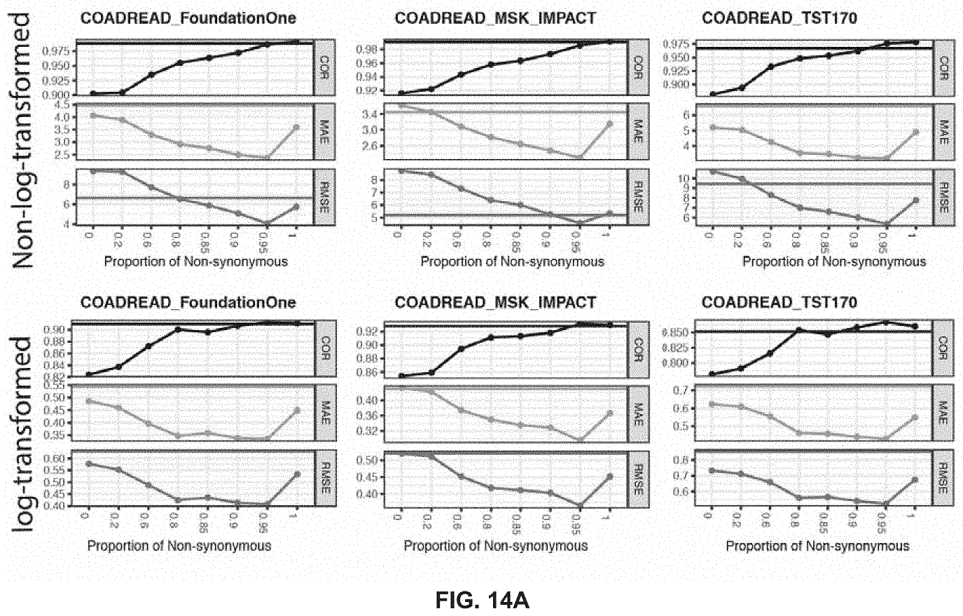

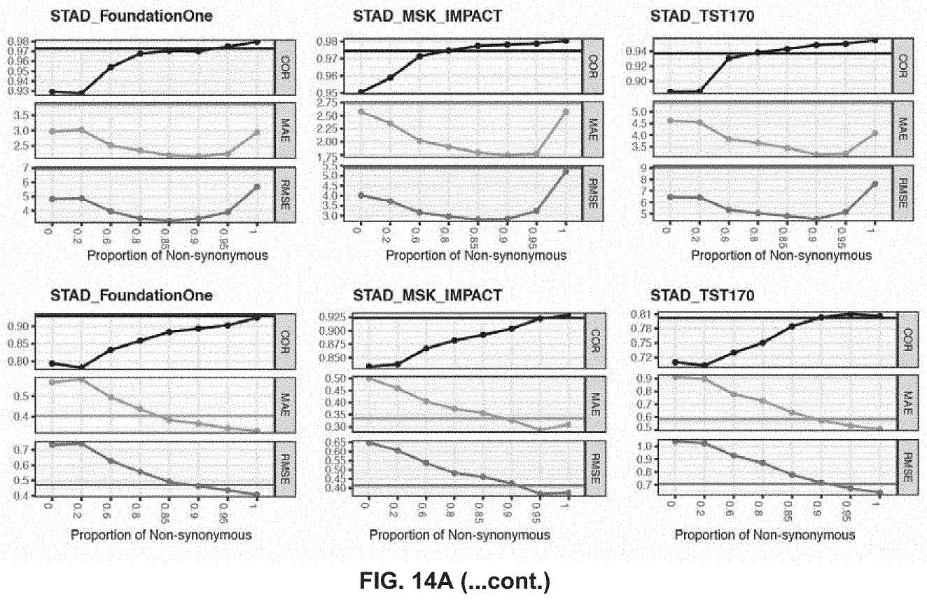

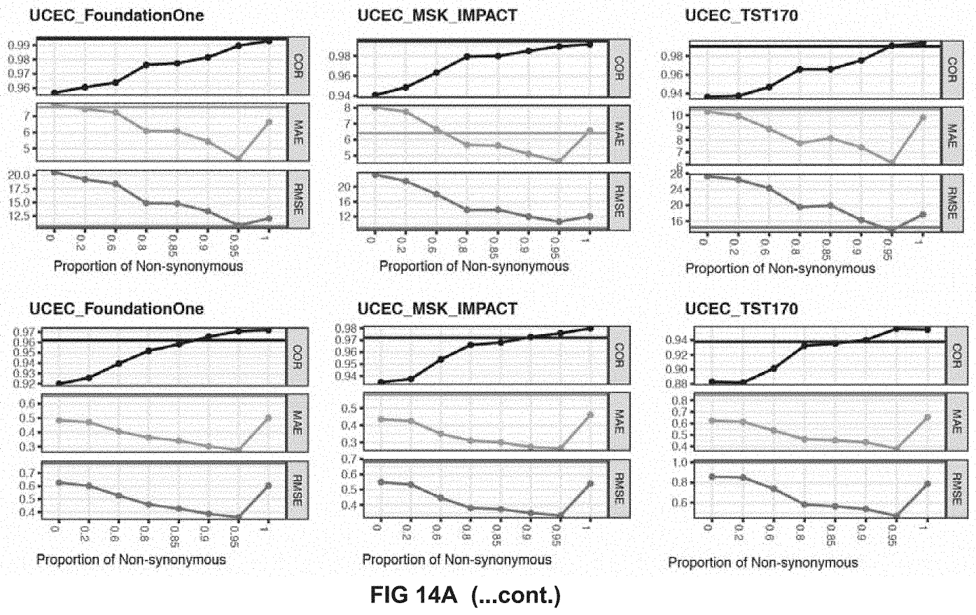

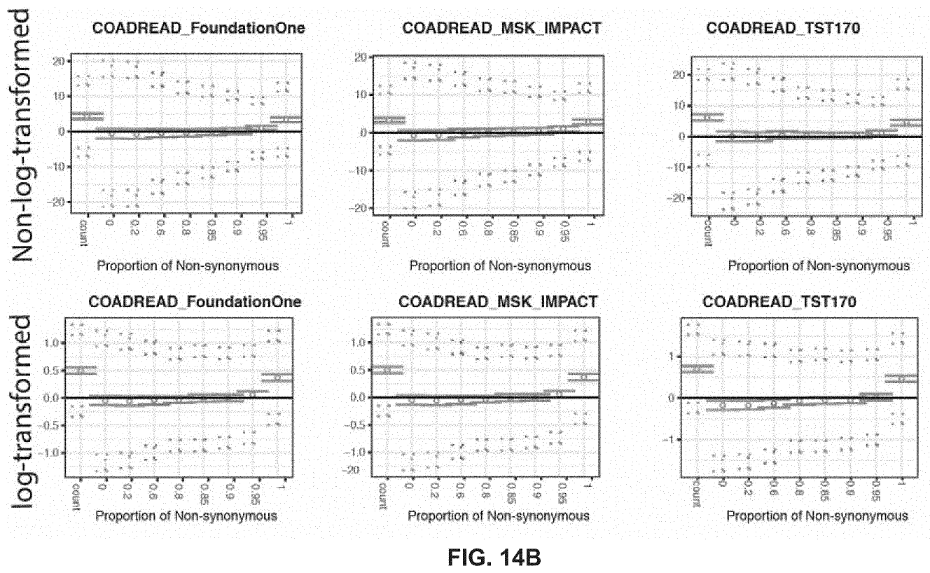

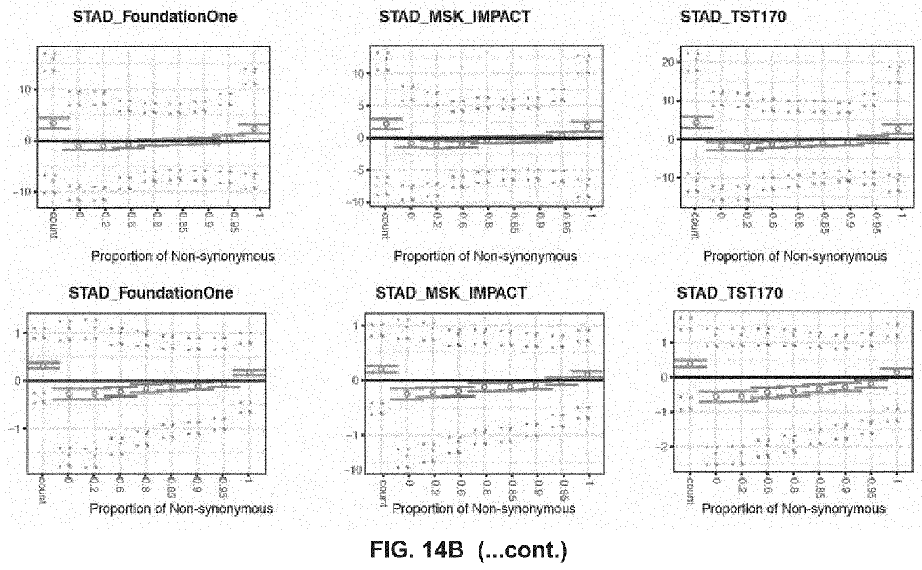

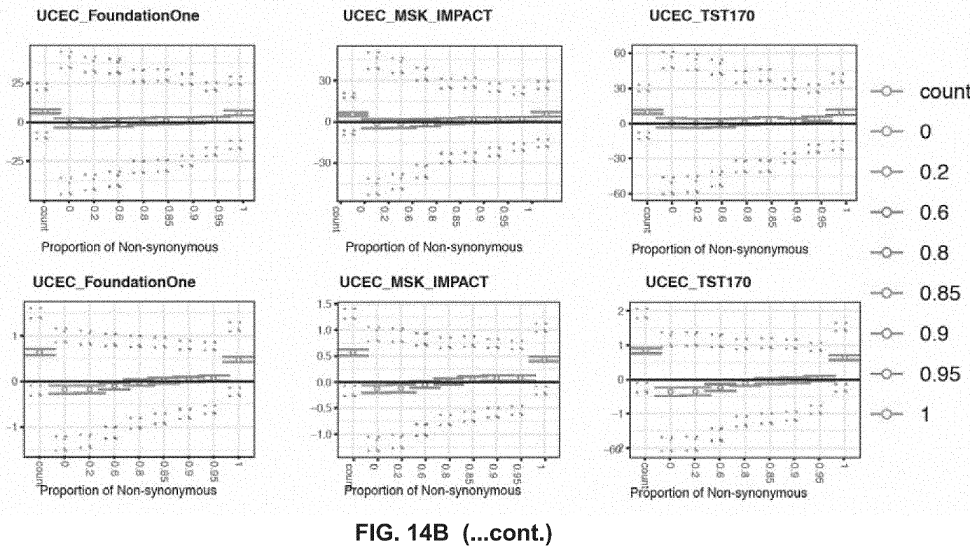

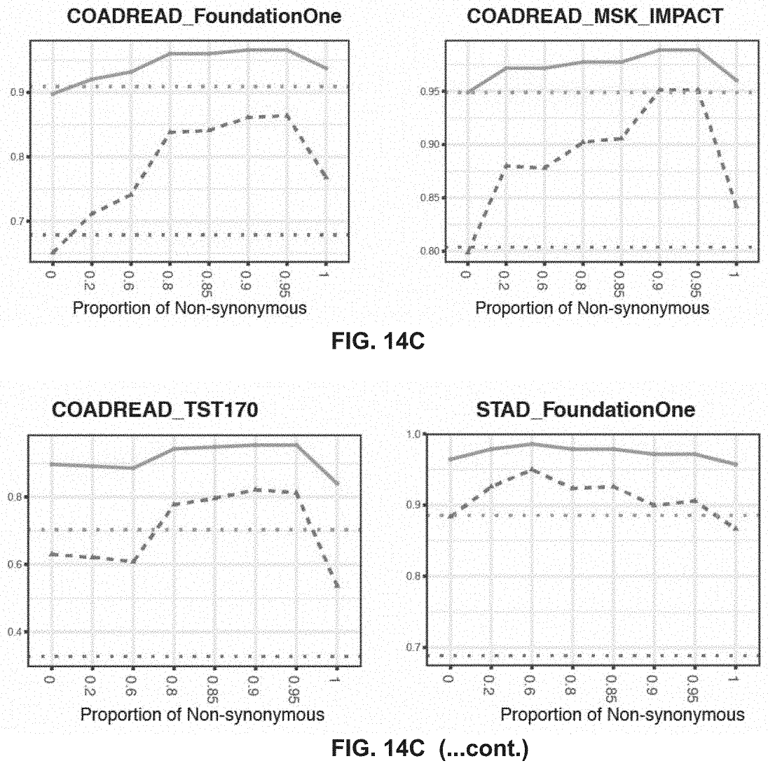

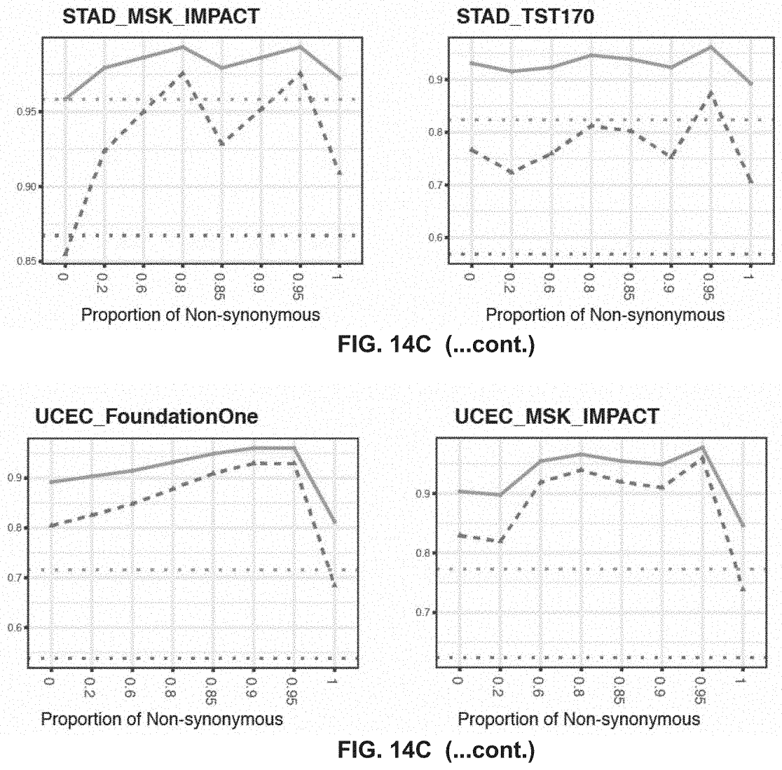

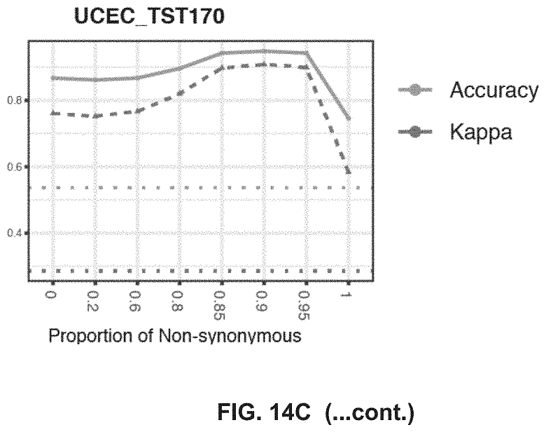

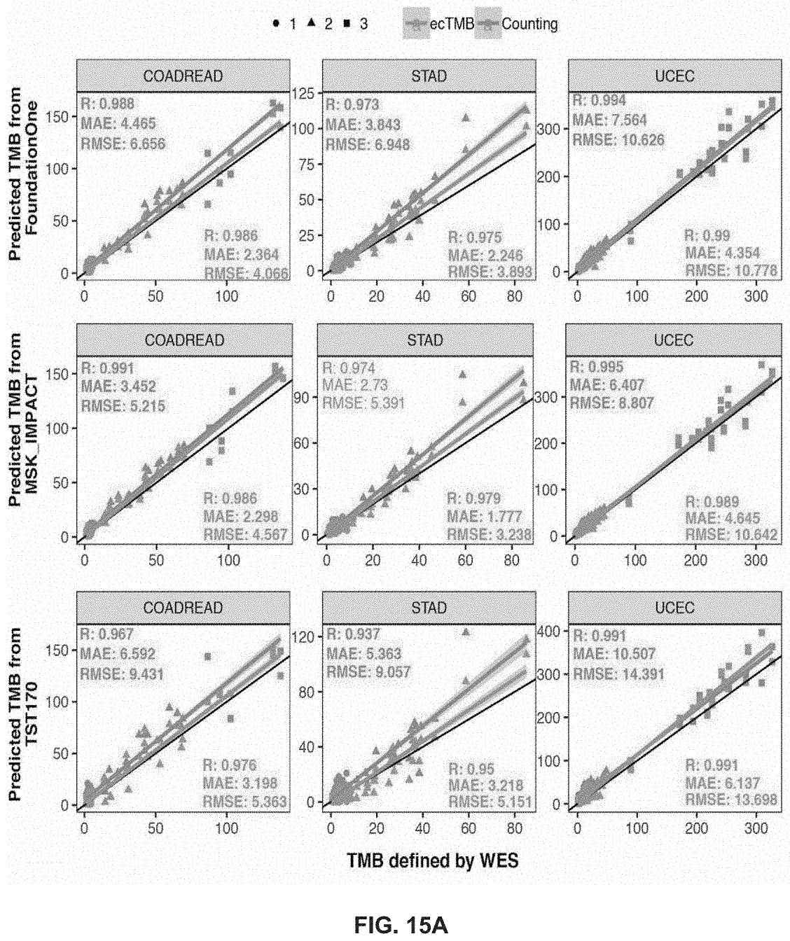

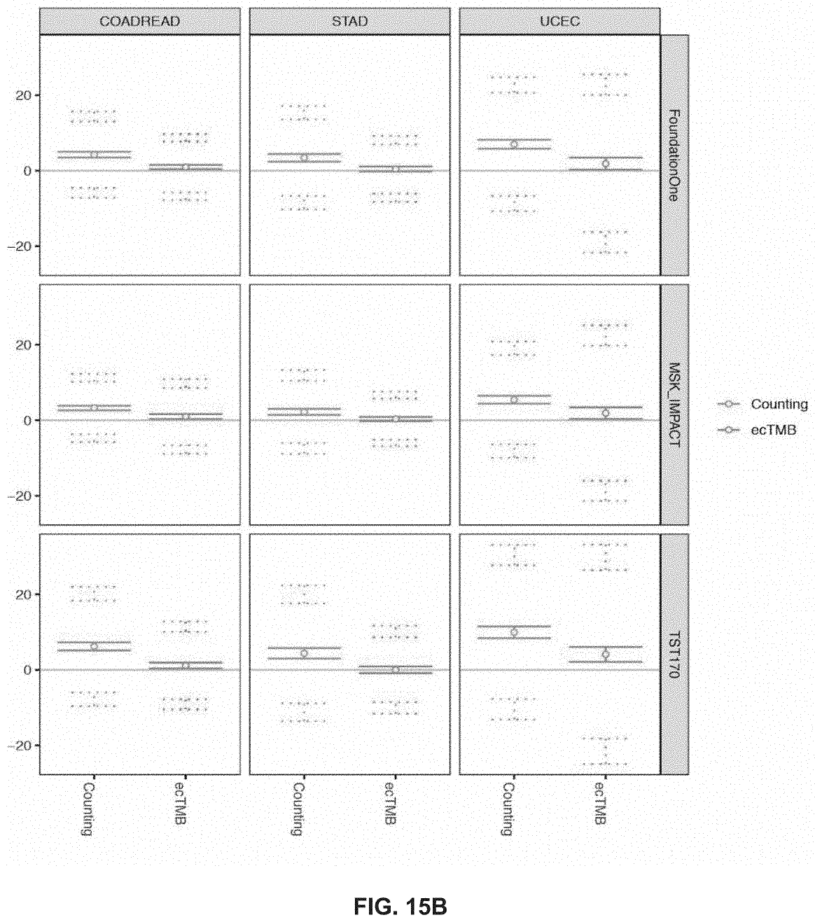

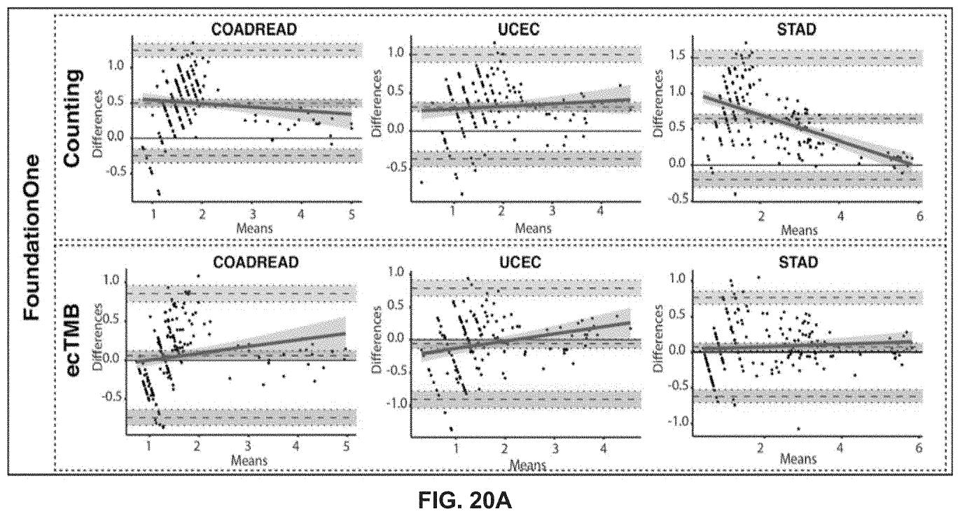

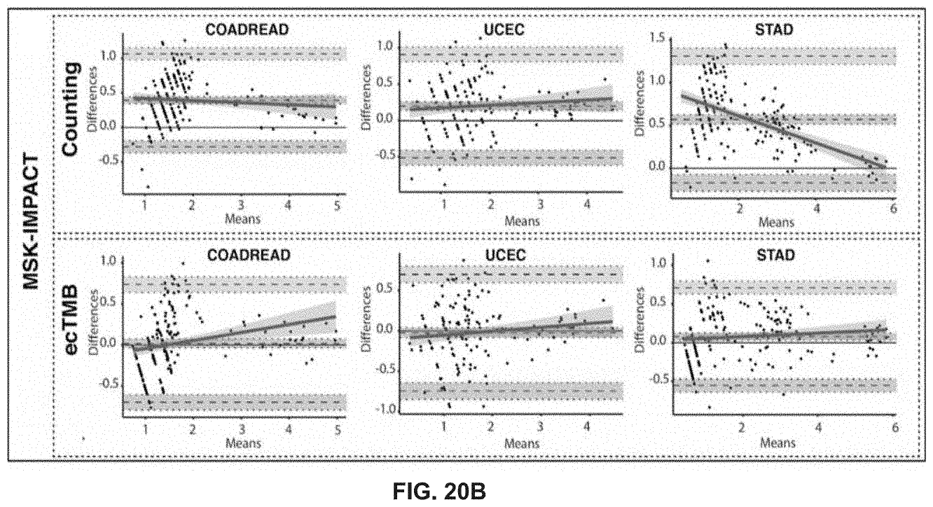

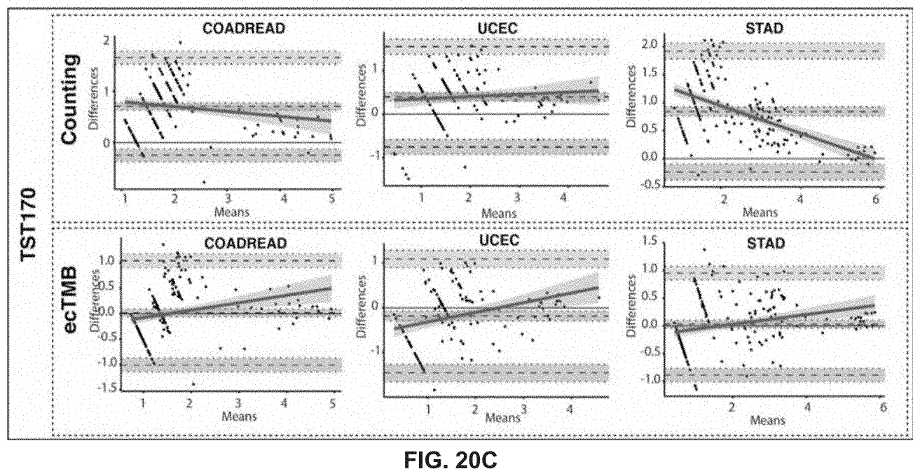

Samples with low TMB tended to be more vulnerable to over-estimation since fewer background mutations led to a higher representation of cancer-associated mutations in counting. In contrast, ecTMB predictions, using synonymous and 95% of non-synonymous mutations, not only had comparable or improved correlation coefficients with WES-based TMB, but also reduced MSE, RMSE and biases. As an example, for the predictions of the TST170 panel in endometrial cancer, ecTMB improved correlation coefficient from 0.938 to 0.956, reduced MAE from 0.848 to 0.381 and removed bias (mean difference changed from 0.03 with 95% confidence interval [−0.04, 0.1] to 0.84 with 95% confidence interval [0.76, 0.92]), when compared with counting prediction (FIG. 22). Each individual Bland-Altman analysis plot can be found in (FIG. 20). The reasons for using 95% of non-synonymous mutations were that 1) fewer synonymous mutations detected within each panel led to less accurate predictions; 2) too many driver gene mutations resulted to prediction biases (FIG. 14). In fact, the mean number of synonymous mutations in colorectal cancer were 4.83, 5.67, 3.55 for FoundationOne, MSK-IMPACT and TST170 panel respectively.

Due to the small size of the panel, the mean number of synonymous mutation mutations per patient in colorectal cancer were 4.83, 5.67, 3.55 for FoundationOne, MSK-IMPACT and TST170 panel, respectively. It was believed to be challenging to generate a robust TMB prediction compared to WES data with thousands of mutations per patient.

Therefore, a series analyses adding different proportion of non-synonymous mutations for panel-based TMB prediction were conducted. Genes were ranked based on mutation frequency in training sets in each cancer types and non-synonymous mutations from the least mutated genes (bottom 0%, 20%, 60%, 80%, 85%, 90%, 95% and 100%) were added to the prediction. Result indicated that the more mutations added, the more accurate it will be. However, prediction bias become a severe problem when the 5% most frequently mutated genes' non-synonymous mutations were added, which are most driver mutations. Therefore, 95% of non-synonymous mutations were used in addition to all synonymous mutations.

Three Cancer Subtypes Revealed by Log-Transformed TMB

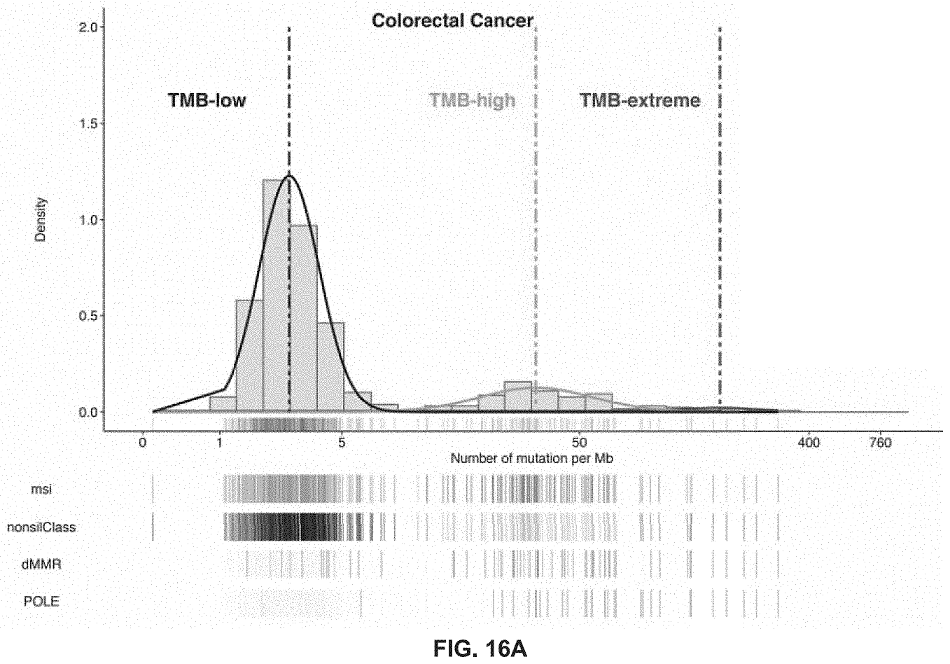

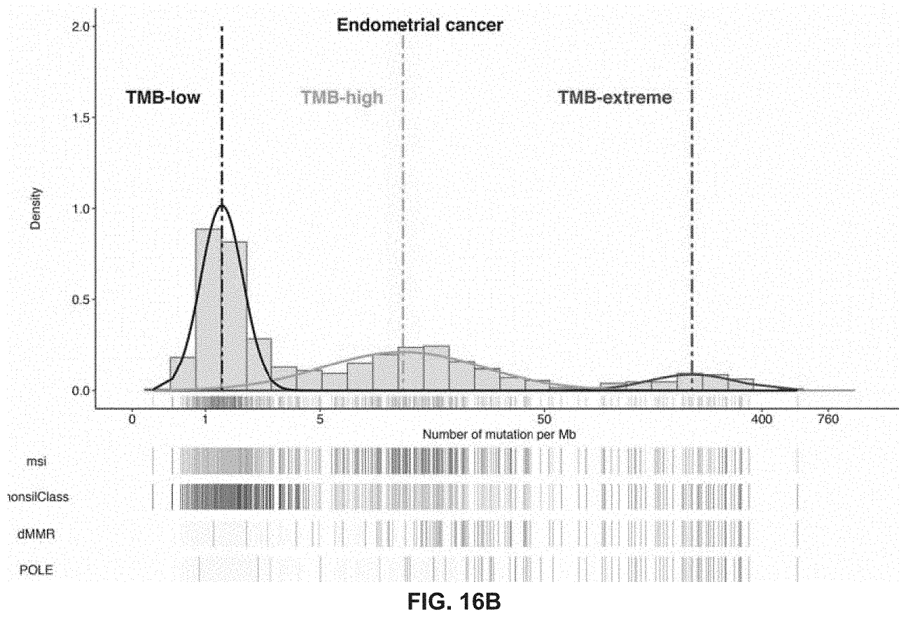

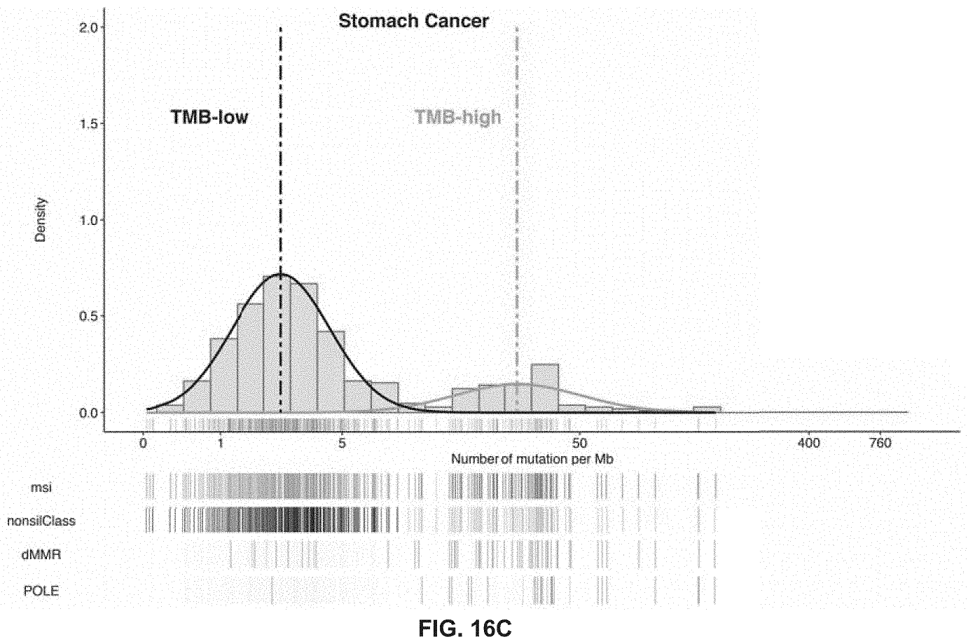

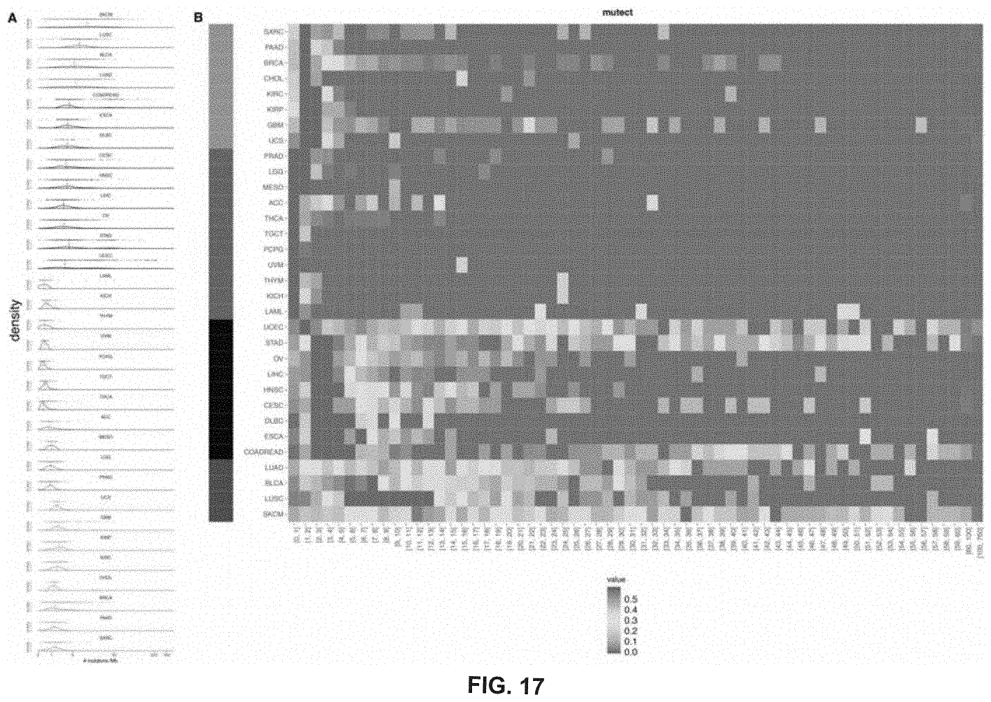

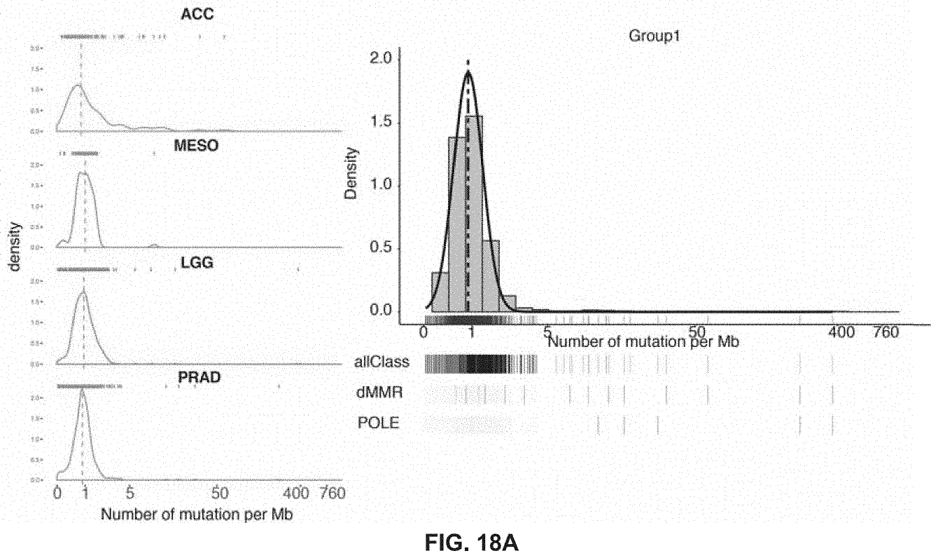

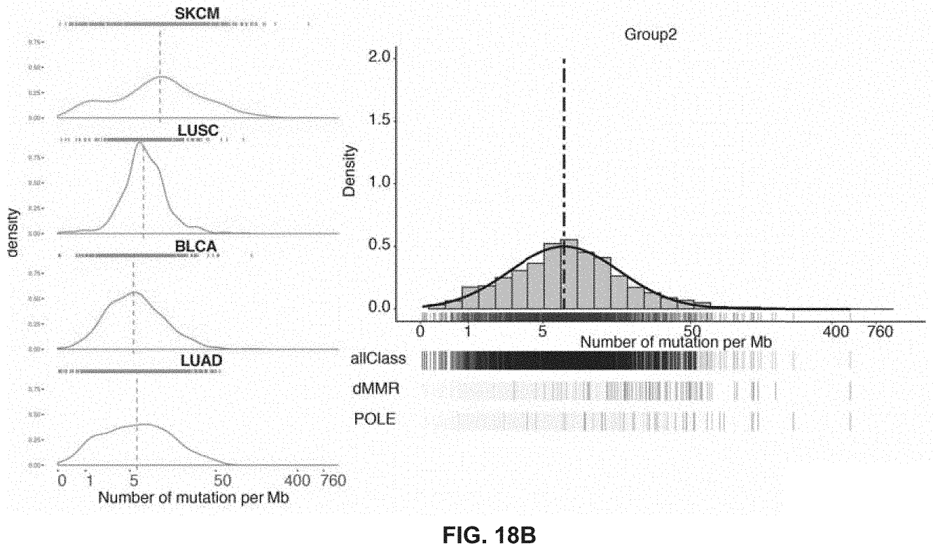

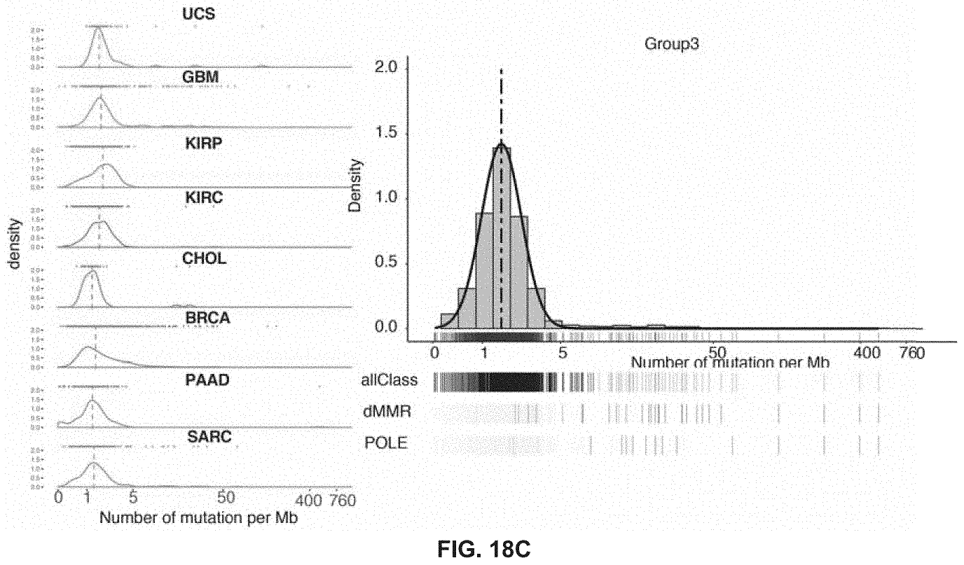

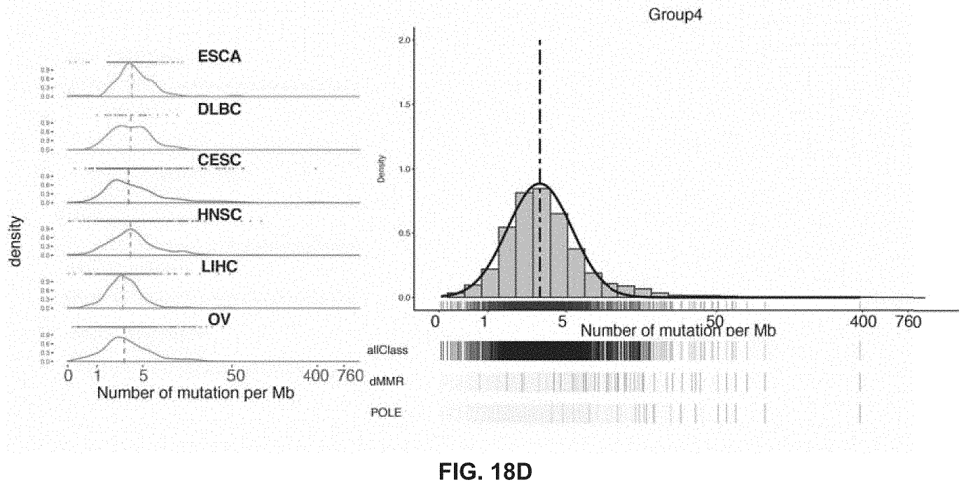

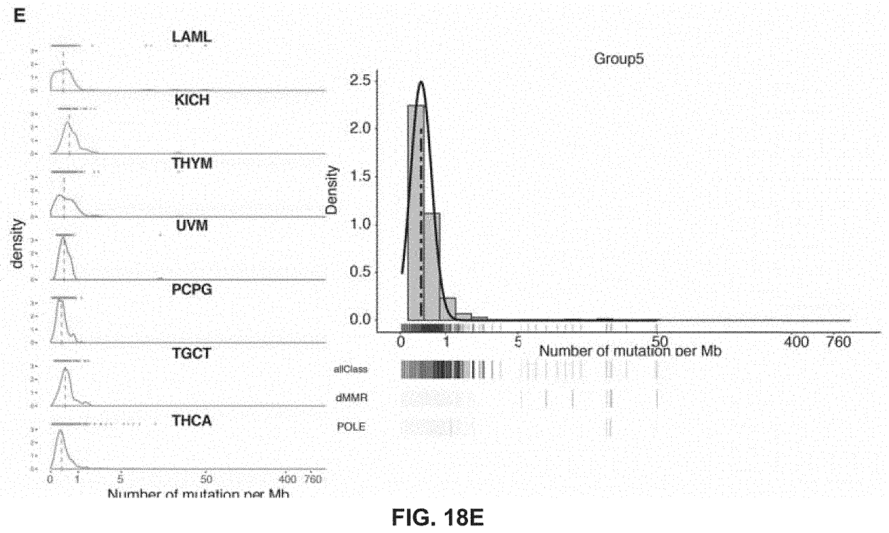

While exploring the distribution of TMB, it was discovered that the distribution of log-transformed WES-based TMB, defined by either the number of all mutations per Mb or the number of non-synonymous mutations per Mb, resembled a mixture of gaussians in colorectal, stomach and endometrial cancers (FIGS. 6A-6C and 16). The investigation of this phenomenon was extended to all cancer types in TCGA. However, it is believed that many cancer types do not have a significant number of hypermutated samples, such as adrenocortical carcinoma (ACC). In order to have a large population of hypermutated samples, we considered aggregating cancer types together. It was discovered, however, that the mutation spectra among cancer types was different, indicating a different threshold for hypermutated population for each cancer. For example, the median mutation rate of skin cutaneous melanoma (SKCM) is about 10 mutations per Mb; and the median of acute myeloid leukemia (LAML) is less than 1 mutation per Mb. Therefore, it was decided to cluster cancer types based on the similarity of the log-transformed TMB distribution (FIG. 17) such that the distribution of log-transformed TMB within each group could be checked. However, the same pattern could not be identified in those groups, which it was believed could have been due to very few hypermutated samples, such as groups 1 and 5, or environmental factors which could cause a continuous mutation spectrum, such as group 2 consisting of SKCM, lung squamous cell carcinoma (LUSC), lung adenocarcinoma (LUAD) and bladder urothelial carcinoma (BLCA) (FIG. 18). Because of the lack of clear subtypes based on log-transformed data in those cancer types, the analyses was focused only on colorectal, stomach and endometrial cancers.

It was found that these three cancer types had the first two Gaussian clusters which consisted of low and high TMB samples respectively. In colorectal and endometrial cancers, there was a third Gaussian cluster, in which samples possessed extremely high TMB. These three hidden subtypes were called as TMB-low, TMB-high and TMB-extreme. Each sample was further classified into these three subtypes using the Gaussian Mixture Model (GMM) within each cancer type to further investigate the biological and clinical significance of these subtypes.

It was believed that the hypermutated phenotype could be caused by mutated POLE or MMR system defects. To gain insights on which mechanism may be responsible for the distinct TMB levels among three subtypes, the non-synonymous mutations in POLE and seven MMR genes were examined, and MSI status detected as described in earlier works (see Network, T. C. G. A. Comprehensive molecular characterization of human colon and rectal cancer. Nature 487, 330-337 (2012); Cui, J. et al. Comprehensive characterization of the genomic alterations in human gastric cancer. Int. J. Cancer 137, 86-95 (2015); and Cancer Genome Atlas Research Network et al. Integrated genomic characterization of endometrial carcinoma. Nature 497, 67-73 (2013)). It was discovered that almost all of the TMB-high samples, 94%, 78% and 91% in colorectal, endometrial and stomach cancers respectively, were MSI-High (MSI-H). A large fraction (92%) of TMB-extreme samples possessed at least one non-synonymous mutation in POLE in both colorectal and endometrial cancers. It was observed that relatively fewer MSI-H cases in TMB-extreme subtype and fewer mutated POLE cases in TMB-high subtype (FIGS. 6A-6C). It was believed that this could be due to the mutually exclusive mechanisms for genomic instability. In previous studies (Govindan, R. et al. Genomic landscape of non-small cell lung cancer in smokers and never-smokers. Cell 150, 1121-1134 (2012)), MMR system defects have been linked to increased deletion/insertion (INDEL), which led us to explore INDEL rates among subtypes. It was discovered that TMB-high samples generally had a significantly higher fraction (˜17%) of INDEL mutations in contrast to what we observed in both TMB-low (˜5%) and TMB-extreme (˜1%) samples (FIGS. 6A-6C). These distinct mutation profiles suggested that the three subtypes defined by log-transformed TMB not only described various levels of TMB, but also represented different biological causes to mutational heterogeneity for patients within the same cancer, where MMR system defects (MSI-H phenotype) were the likely cause for TMB-high and mutated POLE system for TMB-extreme.

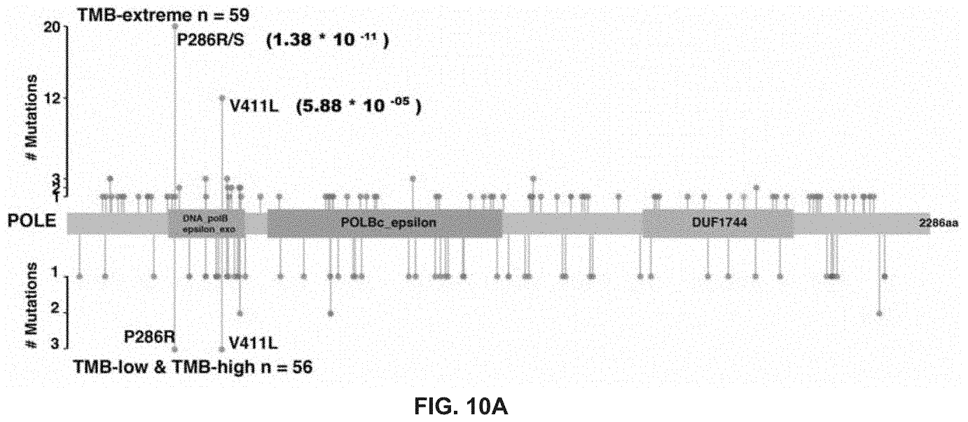

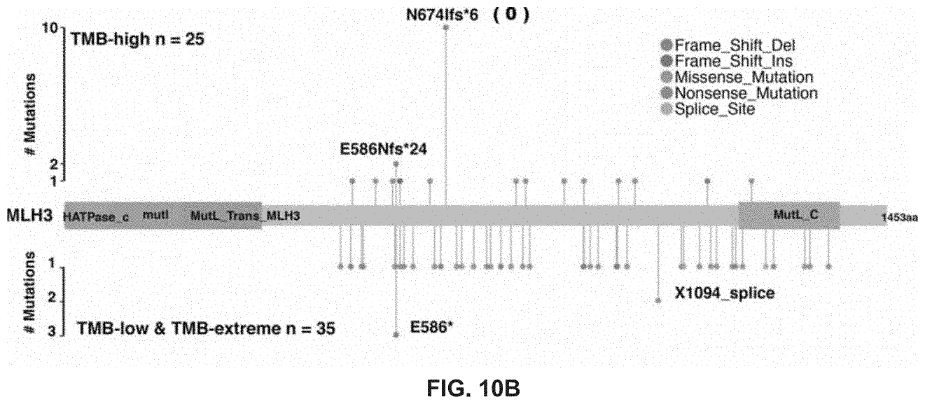

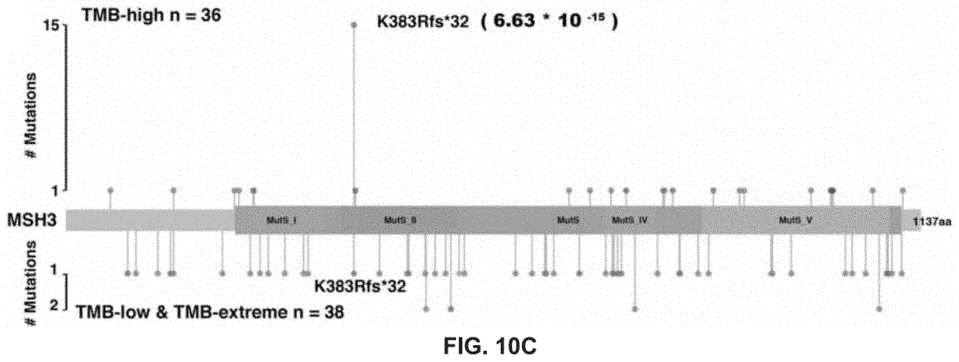

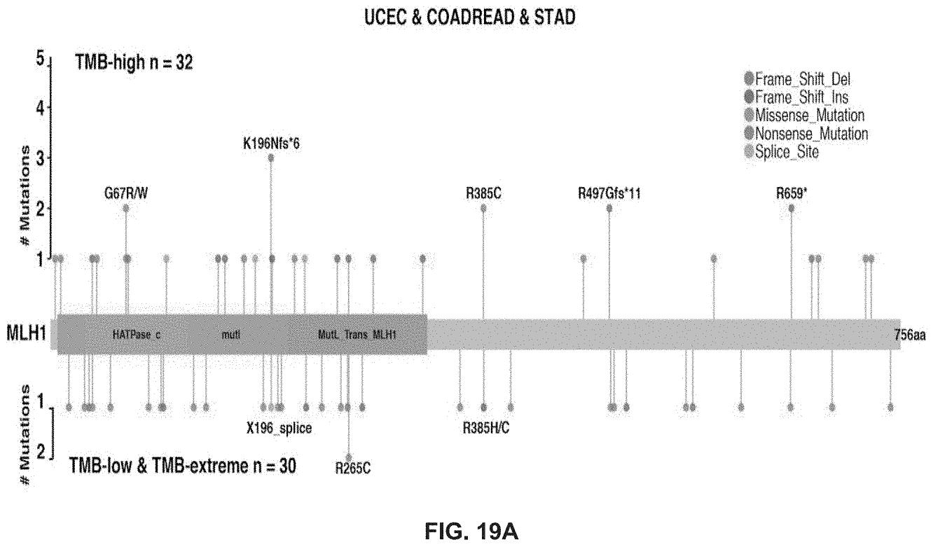

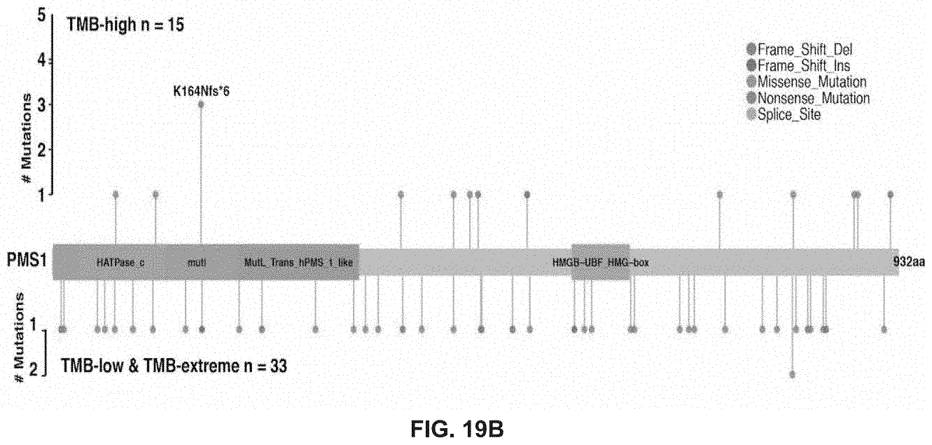

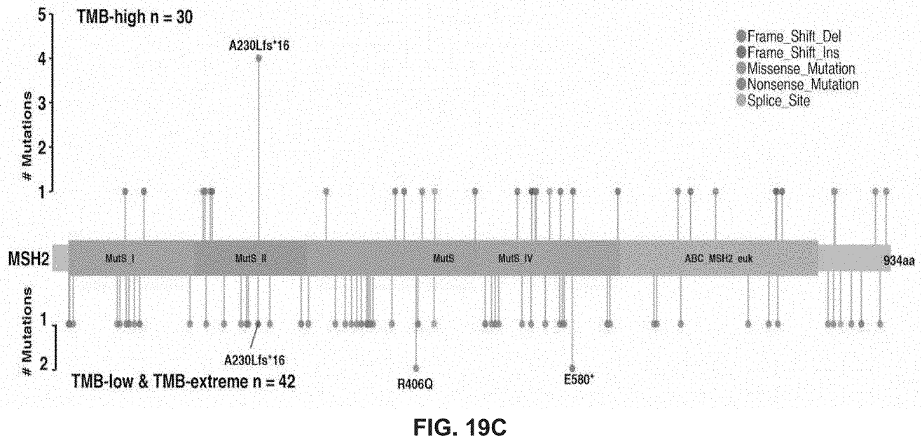

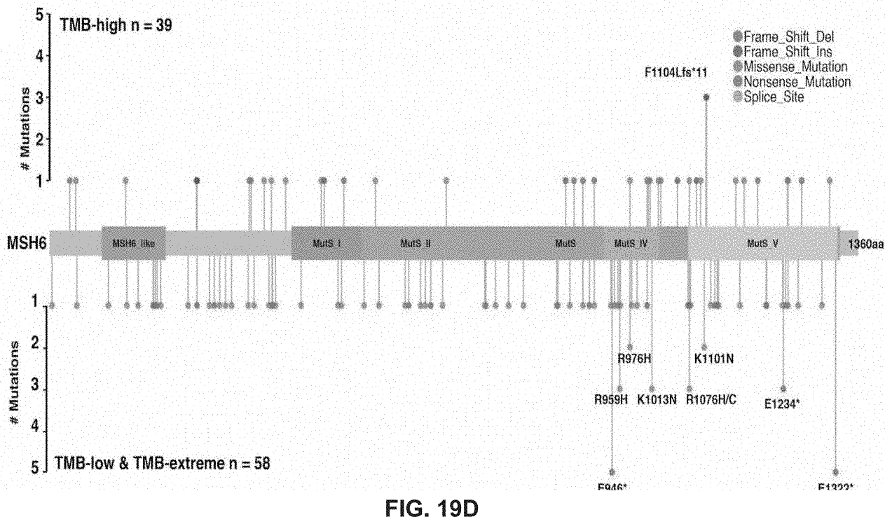

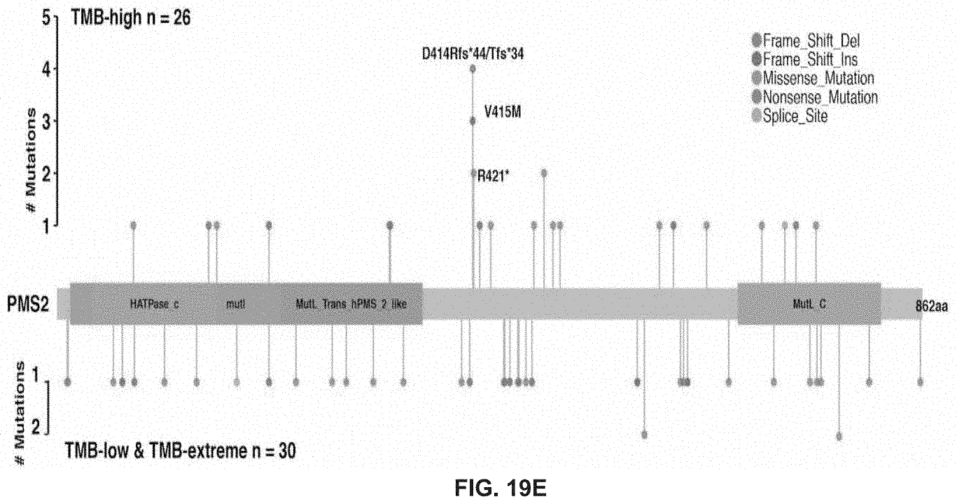

It was believed that not all non-synonymous mutations had a deleterious impact on protein function. In fact, non-synonymous mutations of the POLE gene in TMB-low and TMB-high subtypes and non-synonymous mutations of the MMR system in TMB-low and TMB-extreme subtypes were observed. Therefore, in order to investigate whether any driver mutations can result in TMB-high and TMB-extreme phenotypes, non-synonymous mutations in POLE of TMB-extreme samples were compared against the rest; and we also compared non-synonymous mutations in seven MMR genes of TMB-high samples against the rest, using aggregated colorectal, stomach and endometrial cancer data (FIGS. 10 and 19). As expected, several driver mutations were discovered, including P286R and V411L in POLE, N674lfs*6 in MLH3 and K383Rfs*32 in MSH3 (FIG. 10). P286R and V411L in POLE were known driver mutations which have been linked to the hypermutated phenotype (Campbell, B. B. et al. Comprehensive Analysis of Hypermutation in Human Cancer. Cell 171, 1042-1056.e10 (2017)). Among the 59 TMB-extreme samples which had at least one non-synonymous mutation in POLE, we identified twenty samples with P286R/S and 12 samples with V411L, which were significantly enriched compared to rest of the samples with binomial test p-values 1.38*10-11 and 5.88*10-5 respectively. N674lfs*6 in MLH3 and K383Rfs*32 in MSH3 had been detected in other studies but were never reported as driver mutations for either MSI-H or hypermutation phenotypes (Van Allen, E. M. et al. The genetic landscape of clinical resistance to RAF inhibition in metastatic melanoma. Cancer Discov 4, 94-109 (2014); Mouradov, D. et al. Colorectal cancer cell lines are representative models of the main molecular subtypes of primary cancer. Cancer Research 74, 3238-3247 (2014); Kumar, A. et al. Substantial interindividual and limited intraindividual genomic diversity among tumors from men with metastatic prostate cancer. Nat Med 22, 369-378 (2016); Giannakis, M. et al. Genomic Correlates of Immune-Cell Infiltrates in Colorectal Carcinoma. CellReports 17, 1206 (2016); and Wang, K. et al. Whole-genome sequencing and comprehensive molecular profiling identify new driver mutations in gastric cancer. Nat. Genet. 46, 573-582 (2014)).

In this study, we found 10 out of 25 TMB-high samples, which had at least one non-synonymous mutation in MLH3, had N674lfs*6 mutation in contrast to 0 out of 35 MSH3-mutated samples in TMB-low plus TMB-extreme subtype (p-value=0). Additionally, 15 out of 36 TMB-high MSH3 mutated samples had K383Rfs*32 mutation in comparison with 1 out of 38 MSH3-mutated samples in TMB-low plus TMB-extreme subtype (p-value=6.63*10-15). The high occurrence of these mutations in the TMB-high subtype suggested their potential driver mutation effects in terms of resulting into MSI-H and relatively high TMB phenotypes.

To investigate the clinical relevance of the three subtypes derived by log-transformed TMB, the associations of subtypes with tumor infiltrating immune cell abundance and overall patient survival were examined. In earlier work, Li T. et al. generated a comprehensive resource of immune infiltrates across multiple cancer types using TCGA data (Li, T. et al. TIMER: A Web Server for Comprehensive Analysis of Tumor-Infiltrating Immune Cells. Cancer Research 77, e108-e110 (2017)). The immune infiltrates estimation for TCGA samples was downloaded from https://cistrome.shinyapps.io/timer/ and analyzed the difference of immune infiltrates' abundance among TMB-low, TMB-high and TMB-extreme in colorectal and endometrial cancers, in which the TMB-extreme subtype was detected. TMB-high and TMB-extreme samples were found to have higher abundances of infiltrating CD8 T cell and Dendritic cell (DC). Additionally, the abundance of infiltrating B cell was significantly higher in only TMB-extreme subtype compared to TMB-high and TMB-low. All differences were significant by Wilcoxon rank test in endometrial cancer but not significant in TMB-extreme subtype of colorectal cancer, which may be due to the small sample size (n=12) (FIG. 8). It has been previously noted that the presence of cytotoxic CD8+ T cells, B cell and mature activated DCs in tumor microenvironment is associated with a good clinical outcome in most cancer types (Giraldo, N. A. et al. The clinical role of the TME in solid cancer. Br. J. Cancer 120, 45-53 (2019)), suggesting TMB-high and TMB-extreme subtypes might have better overall survival outcomes. Because of the small size of the TMB-extreme group in colorectal cancer, a survival analysis on each of aggregated colorectal, stomach and endometrial cancers was conducted. It was discovered that TMB-high and TMB-extreme were associated with improved patient survival at different levels after considering age and cancer stage (hazard ratio (HR) for TMB-high=0.8 with p-value=0.1; hazard ratio (HR) for TMB-extreme=0.32 with p-value=0.006) (FIGS. 7A and 7B), suggesting log-transformed TMB subtypes are clinically relevant.

Classification Performance

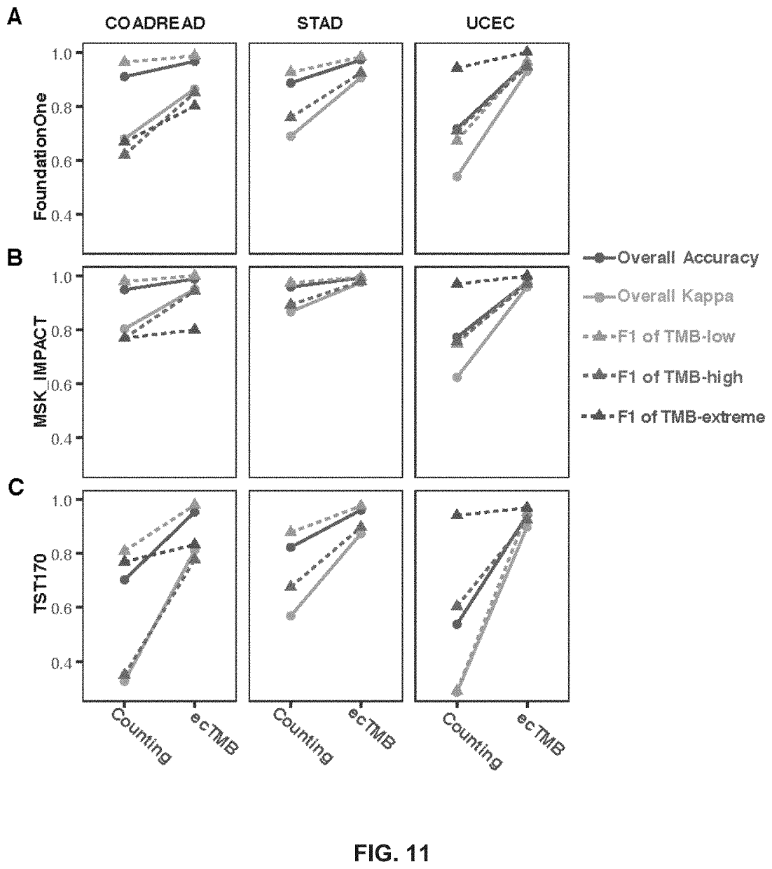

With the discovery of biologically and clinically meaningful subtypes defined by log-transformed TMB, we extended our method to classify TMB subtypes using GMM (FIGS. 5A-5C). Using the subtypes determined by WES-based TMB as truth, we evaluated the classification accuracy using panel-based TMB predicted by ecTMB and the counting method in the testing sets. Comparing against the counting method, classification using ecTMB improved not only the overall accuracy and kappa concordance score, but also F1 score for each subtype classification (FIG. 11).

### DISCUSSION

TMB is an emerging biomarker for cancer immunotherapy and prognosis. However, a lack of consistency on TMB measurement among assays and a lack of meaningful thresholds for the classification of TMB subtypes has become a hurdle for its use as a clinical decision-making biomarker. In our studies, we described a powerful and flexible statistical framework for not only predicting accurate and consistent TMB measurements for various assays but also for classifying samples to one or more TMB subtypes which are believed to be biologically and clinically relevant.

TMB is considered representative of the amount of neo-antigens in tumor since it is historically calculated by counting number of non-synonymous mutation per Mb genome wide. It is believed that TMB is a sample-specific BMR since the majority of mutations are passenger mutations in the whole exome. Thus, based on this second observation, we are the first to implement an explicit background mutation model for TMB prediction. Our background mutation model takes account known mutational heterogeneous factors, including tri-nucleotide context, gene composition, sample mutational burden, gene expression level, and replication timing, and unknown factors through a Bayesian framework. It has been shown that the method improved the background mutation model and successfully predicted synonymous/non-synonymous background mutations, bringing several well-known cancer-specific driver genes to light. Compared to the counting method that simply enumerates the number of observed mutations within sequenced regions per Mb, ecTMB has several advantages.

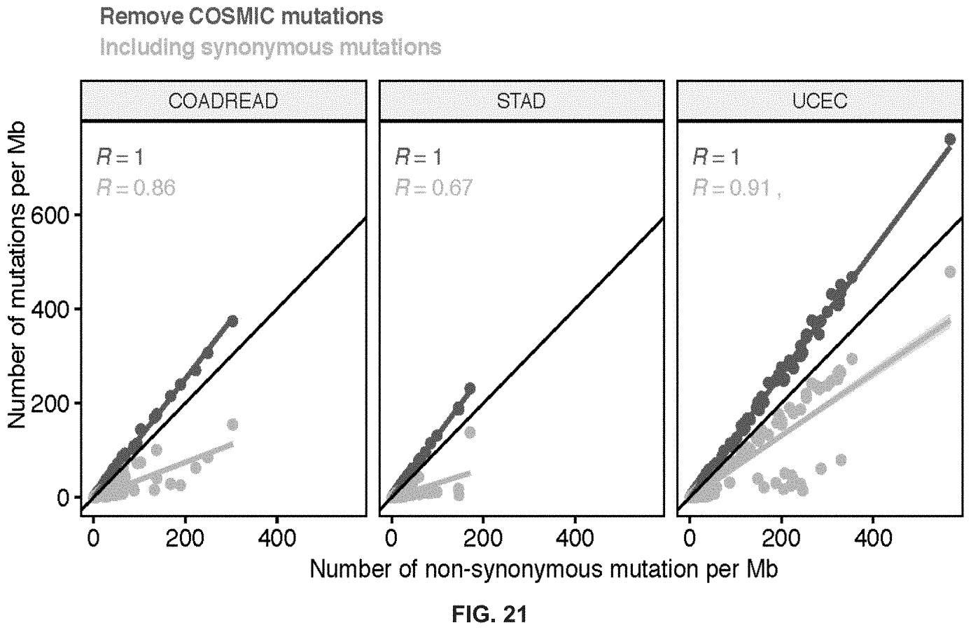

Firstly, ecTMB improves the consistency of TMB prediction among assays. The counting method for TMB prediction, on the other hand, varies with different assays, e.g. FoundationOne CDx, MSK-IMPACT and TST170 and with different kinds of mutation included for prediction. For example, 1) a higher TMB will be detected in targeted panel sequencing as a result of the high enrichment of driver mutations and from mutation hot spots in the cancer target panels, whose mutation rates are normally higher than BMR (FIGS. 14 and 22); 2) removing driver mutations reported by COSMIC may lead to a lower TMB; 3) incorporating synonymous mutations will lead to a higher TMB. Even though these numbers are highly correlated with WES-based TMB (FIG. 21), the fixed or proportional biases can cause inconsistencies among assays. However, ecTMB is able to predict consistent TMB values in a better agreement with the WES-based TMB despite different panels used, whether synonymous mutations are incorporated, or the proportion of non-synonymous mutations used as shown in this study.

Secondly, ecTMB enables the integration of synonymous mutations for TMB prediction. Although panel-targeted sequencing is desirable in clinical practice due to lower costs and fewer DNA input requirements, the cost is that a reduced number of mutations per patient will be detected. The integration of synonymous mutations has the potential to improve the accuracy of panel-based TMB prediction.

Further, ecTMB predicts TMB by considering each gene as an independent negative binomial process, which provides a more robust prediction as compared with predicting TMB based on a single counting value. Although there are other factors influencing the consistency of TMB among assays, such as sequencing depth and somatic mutation caller, it has been demonstrated that ecTMB can help to improve the stability of TMB measurement when those factors are fixed. Potentially, more factors can be added to our statistical framework to further improve consistency of TMB measurements.

As noted herein, the threshold of TMB classification is a debatable topic and different arbitrary cutoffs for TMB have been used. Many studies have tried to assess the biological and clinical interpretation of TMB subtypes based on these arbitrary cutoffs through analyzing the associations with a well-characterized biomarker (e.g. MSI, survival outcome, or immunotherapy responses). Some studies found an association between MSI-H and high TMB, wherein MSI-H tend to be a subset (Chalmers, Z. R. et al. Analysis of 100,000 human cancer genomes reveals the landscape of tumor mutational burden. 1-14 (2017)). However, there are no conclusive thresholds to define a meaningful TMB subtype to study the association. In our work, we discovered three cancer subtypes simply based on a log-transformed TMB, namely TMB-low, TMB-high, and TMB-extreme.

It has been shown that these subtypes not only describe different levels of TMB but also are linked with various causes of hypermutation and overall patient survival. The first subtype is TMB-low, which has low mutation rate and very few mutations in POLE or MMR defects (MSI-H). The second subtype (TMB-high) is characterized with relatively high TMB, high INDEL mutation rate and high enrichment of MSI-H cases. This subtype is the subset that suffers from MMR system defects leading to MSI-H and relatively high TMB phenotype. Interestingly, two novel driver mutations for MMR defects have been discovered. The final subtype is TMB-extreme which is characterized by an extremely high SNV mutation rate but a low INDEL mutation rate, mutated POLE and few MMR defects. Two known POLE driver mutations in this subtype were also discovered. This suggests that dysfunctional POLE might be the root cause of the TMB-extreme subtype. In all, our work is the first to clearly illustrate the association of MSI-H and high TMB, which MSI-H caused due to MMR defects and is one subtype of hypermutated tumor. The novel TMB-extreme subtype shows even better overall survival outcomes compared to TMB-high (MSI-H) subtype and is significantly associated with several tumor infiltrating lymphocytes (TILs), suggesting that TMB-extreme might be another promising marker to predict patient prognosis or guide cancer treatment. The discovery of three TMB subtypes enabled us to extend ecTMB to classify samples based on predicted TMB values with a Gaussian Mixture Model.

These three distinct subtypes were detected in colorectal, stomach and endometrial cancers, which are known to have a high percentage of MSI-H patients and other cancer types have been reported to have very few MSI-H cases (Hause, R. J., Pritchard, C. C., Shendure, J. & Salipante, S. J. Classification and characterization of microsatellite instability across 18 cancer types. Nat Med 22, 1342-1350 (2016)). Therefore, these subtypes may be unique to cancers with a high percentage of MSI-H cases. Among other cancer types, it was discovered that the majority of cancer types have their own basic mutation rate represented by the first Gaussian (FIG. 18), which might associate with their tissue type. For example, low grade glioma (LGG) has a lower basic mutation rate than esophageal carcinoma (ESCA) (FIG. 18), which might be due to a lower rate of cell proliferation in brain than esophageal tissue. Cancers that have been proven to be associated with environmental factors (e.g., UV, tobacco) have a continuous, broader spectrum of high TMB. Meanwhile, hypermutated samples were detected in the remaining cancer types, which are also characterized by high mutation in POLE and MMR system, suggesting that a combination of other mutational biomarker may help further classify these cancers.

Recent work has identified problems with TMB measurements (Melendez, B. et al. Methods of measurement for tumor mutational burden in tumor tissue. Transl Lung Cancer Res 7, 661-667 (2018)). For example, TMB measurements are inconsistent among assays, require higher cost since special larger panels need to be designed to just capture TMB and have no conclusive threshold for classification, which hinder its application in clinical practice. Here, we described a novel and powerful method to predict TMB and classify samples based on TMB robustly. It presents another interpretation of TMB, which is sample-specific background mutation rate, and sheds light on biologically and clinically relevant TMB subtypes. It is believed that the systems and methods described herein can help facilitate the adoption of TMB as biomarker in clinical diagnostics.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A system for reducing a computational burden of classifying a tumor sample derived from a patient, the system comprising: (i) one or more processors, and (ii) one or more memories coupled to the one or more processors, the one or more memories to store computer-executable instructions that, when executed by the one or more processors, cause the system to perform operations comprising:
(a) receiving an identification of somatic mutations within obtained sequencing data, the sequencing data derived from the tumor sample;
(b) estimating a tumor mutational burden based on the received identified somatic mutations; and
(c) assigning a cancer subtype to the tumor sample based on a transformation of the estimated tumor mutational burden.

2. The system of claim 1, wherein the assignment of the cancer subtype comprises (i) modeling the transformation of the estimated tumor mutational burden as a Gaussian mixture model, where each K^{th} component of the Gaussian mixture model represents one cancer subtype; (ii) computing an assignment score for each K^{th} component of the Gaussian mixture model; (iii) identifying a K^{th} component having a highest assignment score; and (iv) assigning the cancer subtype associated with the identified K^{th} component having the highest assignment score as the cancer subtype of the tumor sample.

3. The system of claim 2, wherein parameters for each K^{th} component are estimated using an expectation-maximization algorithm based on training data.

4. The system of claim 1, wherein the tumor mutational burden is estimated using identified non-synonymous mutations.

5. The system of claim 4, wherein the tumor mutational burden is estimated by dividing a total number of identified non-synonymous mutations by a pre-determined genome size.

6. The system of claim 1, the tumor mutational burden is estimated using identified non-synonymous mutations and identified synonymous mutations.

7. The system of claim 6, wherein the tumor mutational burden is estimated by performing a maximum likelihood estimation using the identified non-synonymous and synonymous mutations and a plurality of pre-determined mutation rate parameters.

8. The system of claim 7, wherein the plurality of pre-determined mutation rate parameters comprise (i) gene-specific mutation rate factors, and (ii) context-specific mutation rates.

9. The system of claim 8, wherein the context-specific mutation rates are selected form the group consisting of (i) tri-nucleotide context specific mutation rates; (ii) di-nucleotide context specific mutation rates, and; (iii) mutation signatures.

10. The system of claim 7, wherein the plurality of pre-determined mutation rate parameters are derived by modeling an observed number of mutations for each gene in a training sample derived from whole-exome sequencing.

11. The system of claim 7, wherein the pre-determined mutation rate parameters are derived by: (i) estimating a background mutation rate using one of a negative binomial regression, a poisson regression, a zero-inflated poisson regression, or a zero-inflated negative binomial regression with consideration of only known influencing factors; (ii) estimating a background mutation rate using single gene analysis with consideration of unknown influencing factors; and (iii) combining the estimates of (i) and (ii) within a Bayesian framework.

12. The system of claim 11, wherein the zero-inflated poisson regression is used for estimating the background mutation rate with consideration of only known influencing factors.

13. The system of claim 11, wherein the zero-inflated negative binomial regression is used for estimating the background mutation rate with consideration of only known influencing factors.

14. The system of claim 1, further comprising instructions for computing an overall survival based on the cancer subtype assigned to the tumor sample.

15. The system of claim 1, wherein the received identified somatic mutations are derived from targeted panel sequencing of nucleic acids derived from the tumor sample.

16. The system if claim 1, wherein the transformation of the estimated tumor mutational burden is calculated by performing a log transform on the estimated tumor mutational burden.

17. A system for identifying cancer subtypes within whole exome sequencing data for a type of cancer, the system comprising: (i) one or more processors, and (ii) one or more memories coupled to the one or more processors, the one or more memories to store computer-executable instructions that, when executed by the one or more processors, cause the system to perform operations comprising:
(a) receiving an identification of somatic mutations within obtained whole exome sequencing data;
(b) estimating a tumor mutational burden based on the received identified somatic mutations;
(c) computing a log-transform of the estimated tumor mutational burden to provide a log-transformed estimated tumor mutational burden; and
(d) identifying the cancer subtypes by modeling the log-transformed estimated tumor mutational burden as a Gaussian mixture model.

18. The system of claim 17, wherein the tumor mutational burden is estimated using identified non-synonymous mutations and identified synonymous mutations.

19. The system of claim 18, wherein the tumor mutational burden is estimated by performing a maximum likelihood estimation using the identified non-synonymous and synonymous mutations and a plurality of pre-determined mutation rate parameters.

20. The system of claim 17, wherein three cancer subtypes are identified within the whole exome sequencing data, wherein the whole exome sequencing data is derived from a population of patients, and wherein one of the three cancer subtypes comprises patients whose sequencing data has at least (i) high SNV mutation rates, and (ii) low INDEL mutation rates.
